SEQUENCE LISTING (12 sequences)

SEQ ID NO: 1 - DNA, 3810 bp, synthetic construct
  misc_feature 1..3810: note = pCDM4 vector
  source 1..3810: mol_type = other DNA
gctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagcctaggatcgagatcgatctcgatcccgcgaaattaatacgactcactataggggaattgtgagcggataacaattcccctctagaaataattttgtttaactttaagaaggagatatacatatggcagatctcaattggatatcggccggccacgcgatcgctgacgtcggtaccctcgagtctggtaaagaaaccgctgctgcgaaatttgaacgccagcacatggactcgtctactagtcgcagcttaattaacctaaactgctgccaccgctgagcaataactagcataaccccttggggcctctaaacgggtcttgaggggttttttgctagcgaaaggaggagtcgacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagccagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggccc

SEQ ID NO: 2 - DNA, 24 bp, synthetic construct
  misc_feature 1..24: note = Synthetic FLAG epitope tag polypeptide
  source 1..24: mol_type = other DNA
gactacaaggatgacgatgacaaa

SEQ ID NO: 3 - protein, 8 aa, synthetic construct
  REGION 1..8: note = Synthetic FLAG epitope tag polypeptide
DYKDDDDK

SEQ ID NO: 4 - DNA, 549 bp, Streptomyces griseofuscus
  source 1..549: mol_type = other DNA
atgaacaccttcagaacagccactgccagagacatacctgatgtagcagcaactcttacggaagccttcgcaactgatccacccacgcagtgggtgttccccgacggtactgccgccgtcagcaggttctttacacatgttgcagatagggttcacacggccggtggtattgttgagctactaccagacagagccgccatgattgcattgccaccacacgtgaggctgccaggagaagctgccgacggaaggcaggcggaaattcagagaaggctggcagacaggcacccgctgacacctcactactacctgctgttttacggagttagaacggcacaccagggttcgggattgggcggaagaatgctggccagattaactagcagagctgatagggacagggtgggtacatatactgaggcatccacctggcgtggcgctagactgatgctgagacatggattccatgctacaaggccactaagattgccagatggacccagcatgtttccactttggagagatccaatccatgatcattctgattag

SEQ ID NO: 5 - protein, 182 aa, Streptomyces griseofuscus
MNTFRTATARDIPDVAATLTEAFATDPPTQWVFPDGTAAVSRFFTHVADRVHTAGGIVELLPDRAAMIALPPHVRLPGEAADGRQAEIQRRLADRHPLTPHYYLLFYGVRTAHQGSGLGGRMLARLTSRADRDRVGTYTEASTWRGARLMLRHGFHATRPLRLPDGPSMFPLWRDPIHDHSD

SEQ ID NO: 6 - DNA, 1170 bp, Thermotoga maritima
  source 1..1170: mol_type = other DNA
atgaaaggatatttcggaccatacggtggccagtacgtaccagaaatattaatgggtgccttagaggagttagaggcagcatacgaggagattatgaaggatgagagcttctggaaggagttcaacgatctactgagggattacgcaggcagaccaacgccattgtactttgccaggagattgtctgagaagtacggcgcccgtgtttacttgaagcgtgaggatctgctgcacactggagcacacaagataaataacgctatcggacaggttttattggccaaattaatggggaagacacgtatcatagccgagacgggagctgggcagcatggagtcgctactgctaccgctgctgccctgttcggaatggaatgtgtgatctacatgggtgaagaggacacaatcagacagaagttgaacgtggagcgtatgaaattattaggggctaaagttgtccctgttaagtctggcagtaggaccttgaaggatgcgatagacgaggctttgagagactggattactaatttacagacaacatattatgttatcggatctgttgttggtccccacccttacccaattatcgtaaggaatttccagaaggttatcggtgaggagaccaagaagcaaataccagaaaaggaaggtcgtttgccagactatatagttgcctgcgtaggcggcggtagcaatgccgcaggtatattttacccattcatagactctggagtaaagctgataggtgttgaggcaggtggcgagggattggagacaggtaaacacgcagcctcgttattaaagggtaaaattggctatttacatggatcgaagacctttgttctacaagatgactggggtcaagtccaagtgagccattcggtgtcagctggtcttgactattcaggagtaggacctgagcatgcttattggagagagacagggaaggttctgtacgacgcagtgactgacgaagaggctttggacgcatttatagagttatcaagactagagggcattatacccgctttagagtcatcgcatgctctagcatatttgaagaagataaatataaaaggtaaggttgtggtggtcaacctatcagggagaggggataaagacctggagtcagtcttaaaccatccatacgtgagagaaagaattagatga

SEQ ID NO: 7 - protein, 389 aa, Thermotoga maritima
MKGYFGPYGGQYVPEILMGALEELEAAYEEIMKDESFWKEFNDLLRDYAGRPTPLYFARRLSEKYGARVYLKREDLLHTGAHKINNAIGQVLLAKLMGKTRIIAETGAGQHGVATATAAALFGMECVIYMGEEDTIRQKLNVERMKLLGAKVVPVKSGSRTLKDAIDEALRDWITNLQTTYYVIGSVVGPHPYPIIVRNFQKVIGEETKKQIPEKEGRLPDYIVACVGGGSNAAGIFYPFIDSGVKLIGVEAGGEGLETGKHAASLLKGKIGYLHGSKTFVLQDDWGQVQVSHSVSAGLDYSGVGPEHAYWRETGKVLYDAVTDEEALDAFIELSRLEGIIPALESSHALAYLKKINIKGKVVVVNLSGRGDKDLESVLNHPYVRERIR

SEQ ID NO: 8 - DNA, 42 bp, synthetic construct
  misc_feature 1..42: note = Synthetic V5 epitope tag polypeptide
  source 1..42: mol_type = other DNA
ggtaagccaattccaaatcctttgttgggtttggactccacc

SEQ ID NO: 9 - protein, 14 aa, synthetic construct
  REGION 1..14: note = Synthetic V5 epitope tag polypeptide
GKPIPNPLLGLDST

SEQ ID NO: 10 - DNA, 5203 bp, synthetic construct
  misc_feature 1..5203: note = pETM6-H10 vector
  source 1..5203: mol_type = other DNA
gaagaattgtgagcggataacaattcccctctagaaataattttgtttaactttaagaaggagatatacatatggcagatctcaattggatatcggccggccacgcgatcgctgacgtcggtaccctcgagtctggtaaagaaaccgctgctgcgaaatttgaacgccagcacatggactcgtctactagtcgcagcttaattaacctaaactgctgccaccgctgagcaataactagcataaccccttggggcctctaaacgggtcttgaggggttttttgctagcgaaaggaggagtcgactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagcctaggatcgagatcgatctcgatcccgcgaaattaatacgactcactacg

SEQ ID NO: 11 - DNA, 1446 bp, Bacillus atrophaeus
  source 1..1446: mol_type = other DNA
atgatgtctgaaaatttgcaattgtcagctgaagaaatgagacaattgggttaccaagcagttgatttgatcatcgatcacatgaaccatttgaagtctaagccagtttcagaaacaatcgattctgatatcttgagaaataagttgactgaatctatcccagaaaatggttcagatccaaaggaattgttgcatttcttgaacagaaacgtttttaatcaaattacacatgttgatcatccacatttcttggcttttgttccaggtccaaataattacgttggtgttgttgcagatttcttggcttctggttttaatgtttttccaactgcatggattgctggtgcaggtgctgaacaaatcgaattgactacaattaattggttgaaatctatgttgggttttccagattcagctgaaggtttatttgtttctggtggttcaatggcaaatttgacagctttgactgttgcaagacaggctaagttgaacaacgatatcgaaaatgctgttgtttacttctctgatcaaacacatttctcagttgatagagcattgaaggttttaggttttaaacatcatcaaatctgtagaatcgaaacagatgaacatttgagaatctctgtttcagctttgaagaaacaaattaaagaagatagaactaagggtaaaaagccattctgtgttattgcaaatgctggtactacaaattgtggtgctgttgattctttgaacgaattagcagatttgtgtaacgatgaagatgtttggttgcatgctgatggttcttatggtgctccagctatcttgtctgaaaagggttcagctatgttgcaaggtattcatagagcagattctttgactttagatccacataagtggttgttccaaccatacgatgttggttgtgttttgatcagaaactctcaatatttgtcaaagacttttagaatgatgccagaatacatcaaggattcagaaactaacgttgaaggtgaaattaatttcggtgaatgtggtatcgaattgtcaagaagattcagagctttgaaggtttggttgtcttttaaagttttcggtgttgctgcttttagacaagcaatcgatcatggtatcatgttagcagaacaagttgaagcatttttgggtaaagcaaaagattgggaagttgttacaccagctcaattgggtatcgttacttttagatacattccatctgaattggcatcaacagatactattaatgaaattaataagaaattggttaaggaaatcacacatagaggtttcgctatgttatctactacagaattgaaggaaaaggttgttattagattgtgttcaattaatccaagaactacaactgaagaaatgttgcaaatcatgatgaagattaaagcattggctgaagaagtttctatttcatacccatgtgttgctgaataa

SEQ ID NO: 12 - protein, 481 aa, Bacillus atrophaeus
MMSENLQLSAEEMRQLGYQAVDLIIDHMNHLKSKPVSETIDSDILRNKLTESIPENGSDPKELLHFLNRNVFNQITHVDHPHFLAFVPGPNNYVGVVADFLASGFNVFPTAWIAGAGAEQIELTTINWLKSMLGFPDSAEGLFVSGGSMANLTALTVARQAKLNNDIENAVVYFSDQTHFSVDRALKVLGFKHHQICRIETDEHLRISVSALKKQIKEDRTKGKKPFCVIANAGTTNCGAVDSLNELADLCNDEDVWLHADGSYGAPAILSEKGSAMLQGIHRADSLTLDPHKWLFQPYDVGCVLIRNSQYLSKTFRMMPEYIKDSETNVEGEINFGECGIELSRRFRALKVWLSFKVFGVAAFRQAIDHGIMLAEQVEAFLGKAKDWEVVTPAQLGIVTFRYIPSELASTDTINEINKKLVKEITHRGFAMLSTTELKEKVVIRLCSINPRTTTEEMLQIMMKIKALAEEVSISYPCVAE

(19) United States
(12) Patent Application Publication
(10) Pub. No.: US 2023/0040398 A1
(43) Pub. Date: Feb. 9, 2023

(54) NITRATED PSILOCYBIN DERIVATIVES AND METHODS OF USING

(71) Applicant: Enveric Biosciences Canada Inc., Calgary (CA)

(72) Inventors: Jillian M. Hagel, Calgary (CA); Peter J. Facchini, Calgary (CA); Chang-Chun Ling, Calgary (CA)

(21) Appl. No.: 17/941,531

(22) Filed: Sep. 9, 2022

Related U.S. Application Data
(63) Continuation of application No. PCT/CA2021/051214, filed on Sep. 2, 2021.
(60) Provisional application No. 63/073,534, filed on Sep. 2, 2020.

(51) Int. Cl. A61K 31/4045; A61P 25/18

(52) U.S. Cl. CPC A61K 31/4045; A61P 25/18

(57) ABSTRACT

Disclosed are novel nitrated psilocybin derivative compounds and pharmaceutical and recreational drug formulations containing the same. The nitrated psilocybin derivative compounds may be chemically synthesized or biochemically synthesized in host cells.

### RELATED APPLICATION

This application is a continuation of PCT Application No. PCT/CA2021/051214 filed Sep. 2, 2021, which claims the benefit of U.S. Provisional Application No. 63/073,534 filed Sep. 2, 2020; the entire contents of Patent Application Nos. PCT/CA2021/051214 and 63/073,534 are hereby incorporated by reference.

### INCORPORATION OF SEQUENCE LISTING

A computer readable form of the Sequence Listing “29664-P62567US02_SequenceListing.xml” (93,886 bytes), submitted via EFS-WEB and created on Sep. 9, 2022, is herein incorporated by reference.

### FIELD OF THE DISCLOSURE

The compositions and methods disclosed herein relate to a chemical compound known as psilocybin. Furthermore, the compositions and methods disclosed herein relate in particular to nitrated forms of psilocybin.

### BACKGROUND OF THE DISCLOSURE

The following paragraphs are provided by way of background to the present disclosure. They are not however an admission that anything discussed therein is prior art or part of the knowledge of a person of skill in the art.

The biochemical pathways in the cells of living organisms may be classified as being part of primary metabolism, or as being part of secondary metabolism. Pathways that are part of a cell's primary metabolism are involved in catabolism for energy production or in anabolism for building block production for the cell. Secondary metabolites, on the other hand, are produced by the cell without having an obvious anabolic or catabolic function. It has long been recognized that secondary metabolites can be useful in many respects, including as therapeutic compounds.

Psilocybin, for example, is a secondary metabolite that is naturally produced by certain mushrooms which taxonomically can be classified as belonging the Basidiomycota division of the fungi kingdom. Mushroom species which can produce psilocybin include species belonging to the genus Psilocybe, such as Psilocybe azurescens, Psilocybe semilanceata, Psilocybe serbica, Psilocybe mexicana, and Psilocybe cyanescens, for example. The interest of the art in psilocybin is well established. Thus, for example, psilocybin is a psychoactive compound and is therefore used as a recreational drug. Furthermore, psilocybin is used as a research tool in behavioral and neuro-imaging studies in psychotic disorders, and has been evaluated for its clinical potential in the treatment of mental health conditions (Daniel, J. et al. Mental Health Clin/, 2017; 7(1): 24-28), including to treat anxiety in terminal cancer patients (Grob, C. et al. Arch. Gen. Psychiatry, 2011, 68(1) 71-78) and to alleviate symptoms of treatment-resistant depression (Cathart-Haris, R. L. et al. Lancet Psychiatry, 2016, 3: 619-627).

Although the toxicity of psilocybin is low, adverse side effects, including, for example, panic attacks, paranoia and psychotic states, sometimes together or individually referred to as “a bad trip”, are not infrequently experienced by recreational psilocybin users.

There exists therefore a need in the art for improved psilocybin compounds.

### SUMMARY OF THE DISCLOSURE

The following paragraphs are intended to introduce the reader to the more detailed description, not to define or limit the claimed subject matter of the present disclosure.

In one aspect, the present disclosure relates to psilocybin and derivative compounds.

In another aspect, the present disclosure relates to nitrated psilocybin derivative compounds and methods of making and using these compounds.

Accordingly, in one aspect, the present disclosure provides, in at least one embodiment, in accordance with the teachings herein, a chemical compound or salt thereof having formula (I):

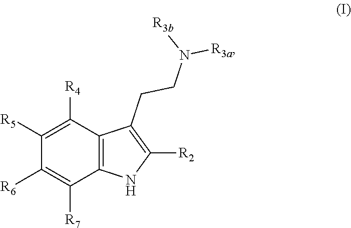

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen atom, an alkyl group, an aryl group, or an acyl group.

In at least one embodiment, in an aspect, R₂ can be a nitro group, R₅, R₆ and R₇ can each be a hydrogen atom or an alkyl, O-alkyl or O-aryl group, and R₄ can be a phosphate group, a hydrogen atom, a hydroxy group, or an alkyl, O-alkyl or O-aryl group.

In at least one embodiment, in an aspect, R₄ can be a nitro group and R₂, R₅, R₆ and R₇ can each be a hydrogen atom or an alkyl, O-alkyl or O-aryl group.

In at least one embodiment, in an aspect, R₅ can be a nitro group, R₂, R₆ and R₇ can each be a hydrogen atom or an alkyl, O-alkyl or O-aryl group, and R₄ can be a phosphate group, a hydrogen atom, a hydroxy group, or an alkyl, O-alkyl or O-aryl group.

In at least one embodiment, in an aspect, Re can be a nitro group, R₂, R₅ and R₇ can each be a hydrogen atom or an alkyl, O-alkyl or O-aryl group, and R₄ can be a phosphate group, a hydrogen atom, a hydroxy group, or an alkyl, O-alkyl or O-aryl group.

In at least one embodiment, in an aspect, R₇ can be a nitro group, R₂, R₅ and R₆ can each be a hydrogen atom or an alkyl, O-alkyl or O-aryl group, and R₄ can be a phosphate group, a hydrogen atom, a hydroxy group, or an alkyl, O-alkyl or O-aryl group.

In at least one embodiment, in an aspect, at least two of R₂, R₄, R₅, R₆ or R₇ can be a nitro group.

In at least one embodiment, in an aspect, R₂ and R₄ can be a nitro group, and R₅, R₆ and R₇ can be a hydrogen atom or an alkyl, O-alkyl or O-aryl group.

In at least one embodiment, in an aspect, R₂ and R₅ can be a nitro group, R₆ and R₇ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₂ and R₆ can be a nitro group, R₅ and R₇ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₂ and R₇ can be a nitro group, R₅ and R₆ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₄ and R₅ can be a nitro group, and R₂, R₆ and R₇ can be a hydrogen atom or an alkyl, O-alkyl or O-aryl group.

In at least one embodiment, in an aspect, R₄ and R₆ can be a nitro group, and R₂, R₅ and R₇ can be a hydrogen atom or an alkyl, O-alkyl or O-aryl group.

In at least one embodiment, in an aspect, R₄ and R₇ can be a nitro group and R₂, R₅ and R₆ can be a hydrogen atom or an alkyl, O-alkyl or O-aryl group.

In at least one embodiment, in an aspect, R₅ and R₆ can be a nitro group, R₂ and R₇ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₅ and R₇ can be a nitro group, R₂ and R₆ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₆ and R₇ can be a nitro group, R₂ and R₅ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₄ when it is not nitrated can be a hydrogen atom.

In at least one embodiment, in an aspect, R₄ when it is not nitrated can be a hydroxy group.

In at least one embodiment, in an aspect, R₄ when it is not nitrated can be an alkyl group.

In at least one embodiment, in an aspect, R₄ when it is not nitrated can be a phosphate group.

In at least one embodiment, in an aspect, three, four or all five of R_{Z}, R₄, R₅, R₆ or R₇ can be a nitro group.

In at least one embodiment, in an aspect, the chemical compound can be selected from the group consisting of compounds having formulas (III); (IV); (V); (VI); (VII); (VIII); (IX); (X); (XXVIII); and (XXIX):

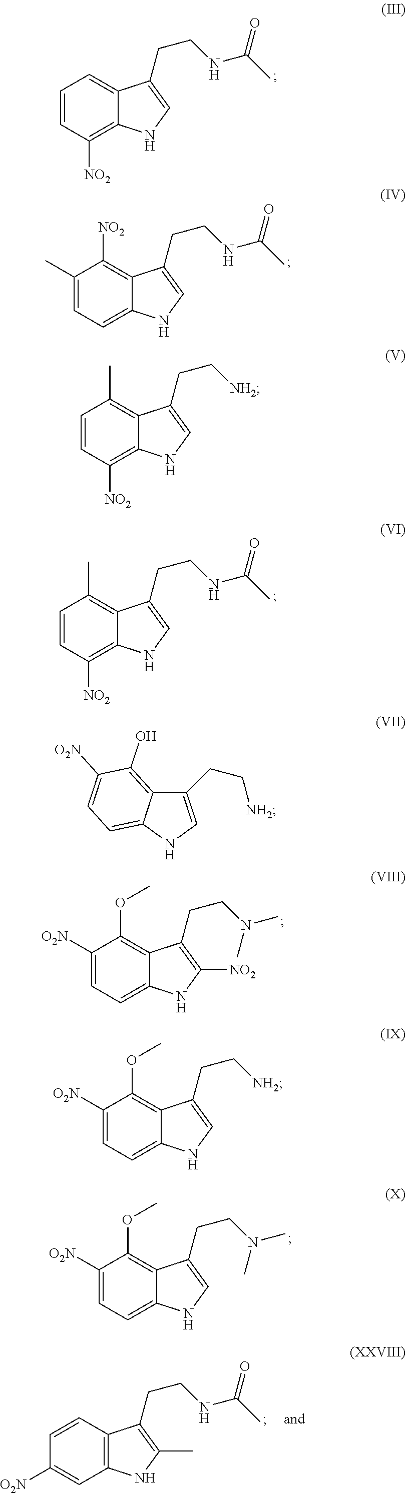

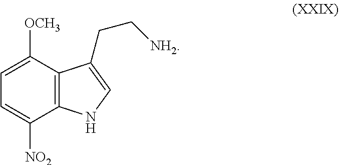 [SMILES: COc1ccc([N+](=O)[O-])c2[nH]cc(CCN)c12]

In at least one embodiment, in an aspect, the chemical compound can be at least about 95% (w/w) pure.

In another aspect, the present disclosure relates to pharmaceutical and recreational drug formulations comprising nitrated psilocybin derivatives. Accordingly, in one aspect, the present disclosure provides, in at least one embodiment, a pharmaceutical or recreational drug formulation comprising an effective amount of a chemical compound or salt thereof having formula (I):

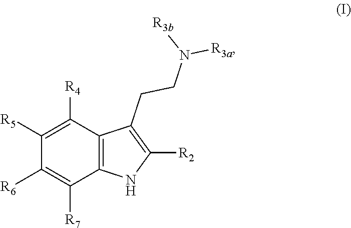

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen atom, an alkyl group, an aryl group, or an acyl group, together with a pharmaceutically acceptable excipient, diluent or carrier.

In another aspect, the present disclosure relates to methods of treatment of psychiatric disorders. Accordingly, the present disclosure further provides, in at least one embodiment, a method for treating a psychiatric disorder, the method comprising administering to a subject in need thereof a pharmaceutical formulation comprising a chemical compound or salt thereof having formula (I):

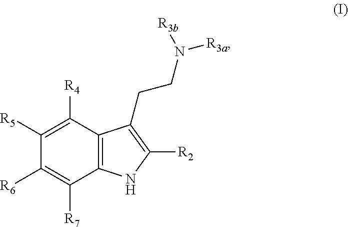

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen atom, an alkyl group, an aryl group, or an acyl group, wherein the pharmaceutical formulation is administered in an effective amount to treat the psychiatric disorder in the subject.

In at least one embodiment, in an aspect, the disorder can be a 5-HT_{2A} receptor mediated disorder.

In at least one embodiment, in an aspect, a dose can be administered of about 0.001 mg to about 5,000 mg.

In another aspect, the present disclosure relates to methods of making nitrated psilocybin derivatives. Accordingly, in one aspect, the present disclosure provides, in at least one embodiment, a method of making a nitrated psilocybin derivative the method comprising:

reacting a reactant psilocybin derivative compound or a salt thereof having the formula (II):

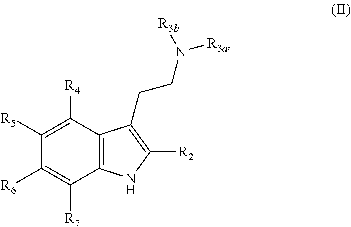

wherein, at least one of R₂, R₄, R₅, R₆, or R₇ is a reactant group, and wherein each R₂, R₅, R₆, or R₇ which is not a reactant group is a hydrogen atom or an alkyl, O-alkyl or O-aryl group, wherein R₄ when it is not a reactant group is a phosphate group, a hydrogen atom, a hydroxy group, or an alkyl, O-alkyl or O-aryl group, and wherein R_{3A} and R_{3B} are a hydrogen atom, an alkyl group, an aryl group or an acyl group, with a nitro group donating compound under conditions sufficient to form a chemical compound having formula (I):

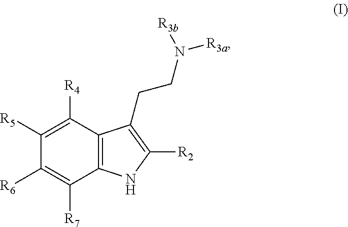

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen atom, an alkyl group, an aryl group, or an acyl group.

In a least one embodiment, in an aspect, the nitro group donating compound can be selected from nitric acid (HNO₃); a nitrate salt; an acyl nitrate; trifluomethansulfonyl nitrate; and trifluoracetyl nitrate.

In another aspect, the present disclosure provides a methods of making a nitrated psilocybin derivative having a chemical compound having formula (I):

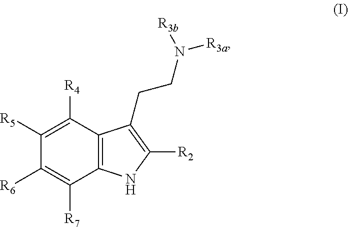

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen atom, an alkyl group, an aryl group, or an acyl group, the method comprising:
(a) reacting a compound having the chemical formula (XI):

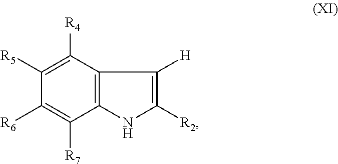

wherein, R₂, R₅, R₆, and R₇ are a hydrogen atom or an alkyl, O-alkyl or O-aryl group, wherein R₄ is a phosphate group, a hydrogen atom, a hydroxy group, or an alkyl, O-alkyl or O-aryl group;
with 1-(dimethylamino)-2-nitroethylene under the catalysis of an acid to form a compound having chemical formula (XII):

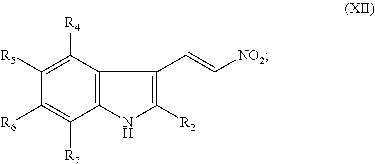

(b) reacting the compound having chemical formula (XII) with sodium borohydride in an alcohol solution to form a compound having formula (XIII):

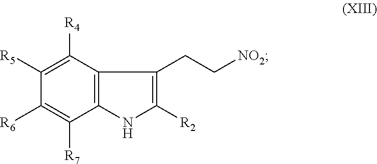

(c) reacting the compound having chemical formula (XIII) under suitable reducing conditions to form a compound having the chemical formula (XIV):

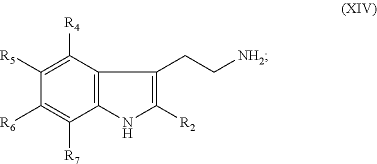

(d) reacting the compound having chemical formula (XIV) with a protecting reagent to form a compound having the chemical formula (XV), or (XVI) or (XVII):

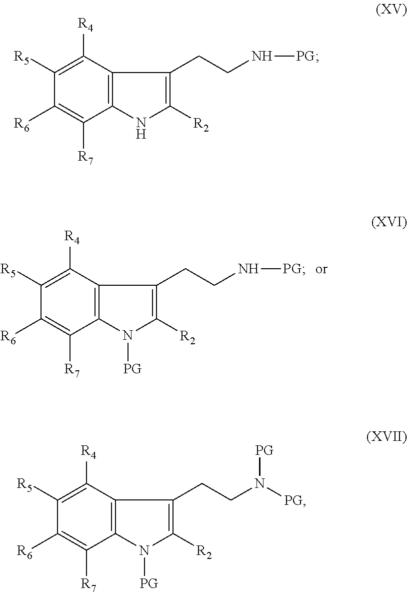

wherein PG is a protecting group;
(e) reacting the compound having chemical formula (XV), (XVI) or (XVII) with a nitro group donating compound to form a compound having the chemical formula (XVIII), (XIX) or (XX):

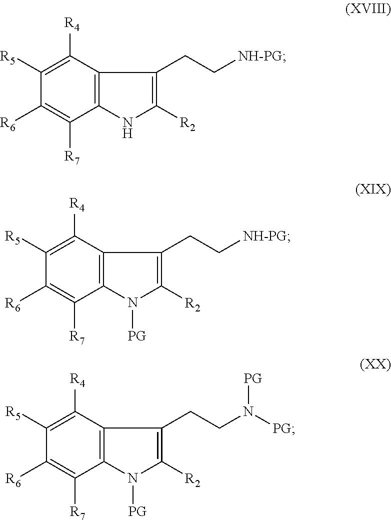

wherein at least one of R₂, R₅, R₆ and R₇ is a nitro group, wherein R₂, R₅, R₆, or R₇ which are not nitrated are a hydrogen atom or an alkyl, O-alkyl or O-aryl group, wherein R₄ when it is not nitrated is a phosphate group, a hydrogen atom, a hydroxy group, or an alkyl, O-alkyl or O-aryl group, and wherein at least one of R₃ₐ and R_{3b} is an alkyl group; and
(f) substituting protective group (PG) in the compound having chemical formula (XVIII), (XIX) or (XX) with a reagent to substitute the protective group to form a compound having the chemical formula (XXI):

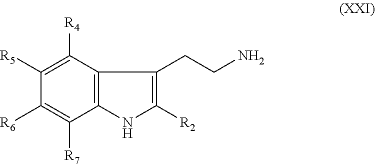

wherein at least one of R₂, R₅, R₆ and R₇ is a nitro group, wherein R₂, R₅, R₆, or R₇ which are not nitrated are a hydrogen atom or an alkyl, O-alkyl or O-aryl group, wherein R₄ when it is not nitrated is a phosphate group, a hydrogen atom, a hydroxy group, or an alkyl, O-alkyl or O-aryl group, and wherein at least one of R₃ₐ and R_{3b} is an alkyl group.

In at least one embodiment, in an aspect, the method can further comprise step (g) comprising reacting the compound having chemical formula (XXI) with (i) an aldehyde or ketone group under reductive amination conditions or (ii) an alkyl electrophile or α,β-unsaturated reagent, to form a compound having the chemical formula (XXII):

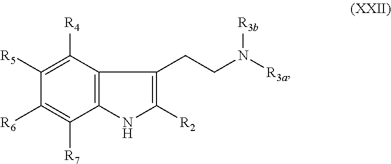

wherein at least one of R₂, R₅, R₆ and R₇ is a nitro group, wherein R₂, R₅, R₆, or R₇ which are not nitrated are a hydrogen atom or an alkyl, O-alkyl or O-aryl group, wherein R₄ when it is not nitrated is a phosphate group, a hydrogen atom, a hydroxy group, or an alkyl, O-alkyl or O-aryl group, and wherein at least one of R₃ₐ and R_{3b} is an alkyl group.

In at least one embodiment, in an aspect, the method can further comprise step (h) comprising reacting the compound having chemical formula (XXII) with an acylating reagent to form a compound having the chemical formula (XXIII):

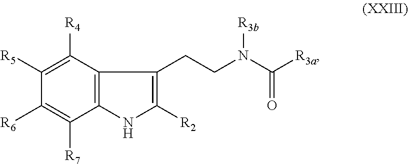

wherein at least one of R₂, R₅, R₆ and R₇ is a nitro group, wherein R₂, R₅, R₆, or R₇ which are not nitrated are a hydrogen atom or an alkyl, O-alkyl or O-aryl group, wherein R₄ when it is not nitrated is a phosphate group, a hydrogen atom, a hydroxy group, or an alkyl, O-alkyl or O-aryl group, and wherein at least one of R₃ₐ and R_{3b} is an alkyl group.

In at least one embodiment, in an aspect, suitable reducing conditions in step (b) can be reacting in the presence of ammonium formate and palladium on charcoal, or lithium aluminum hydride, or sodium borohydride-BF₃.Et₂O.

In at least one embodiment, in an aspect, in step (d) the protecting group can be selected from an alkyl group, an acyl group, or carbamate group.

In at least one embodiment, in an aspect, the nitro group donating compound in step (e) can be selected from AgNO₃-acyl halide, NO₂BF₄, nitric acid-H₂SO₄, and nitric acid-trifluoroacetic acid.

In at least one embodiment, in an aspect, the reagent to substitute the protective group in step (f) can be trifluoroacetic acid in dichloromethane.

In at least one embodiment, in an aspect, two of R₃ₐ and R_{3b} in the compounds having chemical formulas (XII) or (XIII) can be alkyl groups.

In at least one embodiment, in an aspect, R₂ in the compound having formula (I) can be a nitro group, R₅, R₆ and R₇ can each be a hydrogen atom or an alkyl, O-alkyl or O-aryl group, and R₄ can be a phosphate group, a hydrogen atom, a hydroxy group, or an alkyl, O-alkyl or O-aryl group.

In at least one embodiment, in an aspect, R₄ in the compound having formula (I) can be a nitro group and R₂, R₅, R₆ and R₇ can each be a hydrogen atom or an alkyl, O-alkyl or O-aryl group.

In at least one embodiment, in an aspect, R₅ in the compound having formula (I) can be a nitro group, R₂, R₆ and R₇ can each be a hydrogen atom or an alkyl, O-alkyl or O-aryl group, and R₄ can be a phosphate group, a hydrogen atom, a hydroxy group, or an alkyl, O-alkyl or O-aryl group.

In at least one embodiment, in an aspect, Re in the compound having formula (I) can be a nitro group atom, R₂, R₅ and R₇ can each be a hydrogen atom or an alkyl, O-alkyl or O-aryl group, and R₄ can be a phosphate group, a hydrogen atom, a hydroxy group, or an alkyl, O-alkyl or O-aryl group.

In at least one embodiment, in an aspect, R₇ in the compound having formula (I) can be a nitro group, R₂, R₅ and R₆ can each be a hydrogen atom or an alkyl, O-alkyl or O-aryl group, and R₄ can be a phosphate group, a hydrogen atom, a hydroxy group, or an alkyl, O-alkyl or O-aryl group.

In at least one embodiment, in an aspect, at least two of R₂, R₄, R₅, R₆ or R₇ in the compound formula (I) can be a nitro group.

In at least one embodiment, in an aspect, R₂ and R₄ in the compound having formula (I) can be a nitro group, and R₅, R₆ and R₇ can be a hydrogen atom or an alkyl, O-alkyl or O-aryl group.

In at least one embodiment, in an aspect, R₂ and R₅ in the compound having formula (I) can be a nitro group, R₆ and R₇ can be a hydrogen atom, or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₂ and R₆ in the compound having formula (I) can be a nitro group, R₅ and R₇ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₂ and R₇ in the compound having formula (I) can be a nitro group, R₅ and R₆ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₄ and R₅ in the compound having formula (I) can be a nitro group, and R₂, R₆ and R₇ can be a hydrogen atom, or an alkyl, O-alkyl or O-aryl group.

In at least one embodiment, in an aspect, R₄ and R₆ in the compound having formula (I) can be a nitro group, and R₂, R₅ and R₇ can be a hydrogen atom or an alkyl, O-alkyl or O-aryl group.

In at least one embodiment, in an aspect, R₄ and R₇ in the compound having formula (I) can be a nitro group and R₂, R₅ and R₆ can be a hydrogen atom or an alkyl, O-alkyl or O-aryl group.

In at least one embodiment, in an aspect, R₅ and R₆ in the compound having formula (I) can be a nitro group, R₂ and R₇ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₅ and R₇ in the compound having formula (I) can be a nitro group, R₂ and R₆ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₆ and R₇ in the compound having formula (I) can be a nitro group, R₂ and R₅ can be a hydrogen atom or an alkyl, O-alkyl or O-aryl group, and R₄ can be a hydrogen atom, an alkyl, O-alkyl or O-aryl group, a hydroxy group, or a phosphate group.

In at least one embodiment, in an aspect, R₄ in the compound having formula (I) when it is not nitrated can be a hydrogen atom.

In at least one embodiment, in an aspect, R₄ in the compound having formula (I) when it is not nitrated can be a hydroxy group.

In at least one embodiment, in an aspect, R₄ in the compound having formula (I) when it is not nitrated can be an alkyl group.

In at least one embodiment, in an aspect, R₄ in the compound having formula (I) when it is not nitrated can be a phosphate group.

In at least one embodiment, in an aspect, three, four or all five of R₂, R₄, R₅, R₆ or R₇ in the compound having formula (I) can be a nitro group.

In at least one embodiment, in an aspect, the compound having formula (I) can be selected from the group consisting of compounds having formulas (III); (IV); (V); (VI); (VII); (VIII); (IX); (X); (XXVIII); and (XXIX):

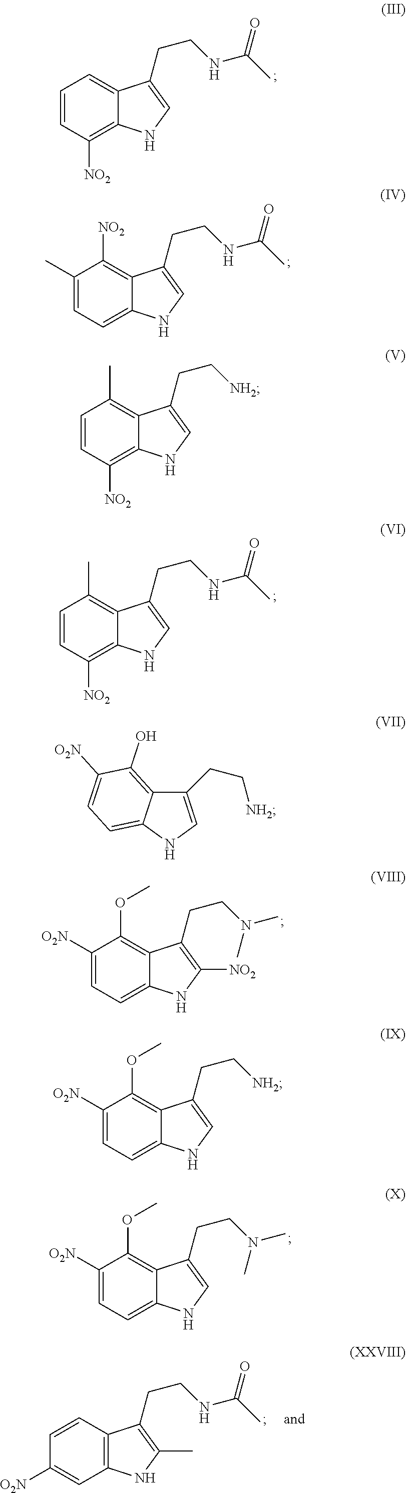

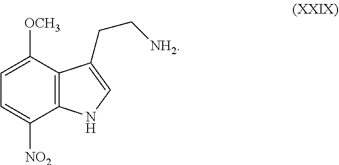 [SMILES: COc1ccc([N+](=O)[O-])c2[nH]cc(CCN)c12]

In another aspect, the present disclosure relates to further methods of making nitrated psilocybin derivatives. Accordingly, in one aspect, the present disclosure provides in at least one aspect, a method of making a nitrated psilocybin derivative the method comprising:
(a) contacting a nitrated psilocybin precursor compound with a host cell comprising a psilocybin biosynthetic enzyme complement; and
(b) growing the host cell to produce a nitrated psilocybin derivative or salts thereof having the formula (I):

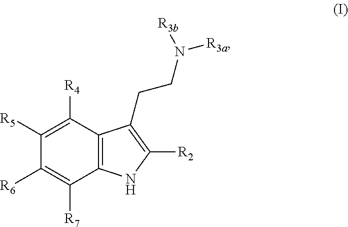

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen atom, an alkyl group, an aryl group, or an acyl group.

In at least one embodiment, in an aspect, the psilocybin biosynthetic enzyme complement can comprise at least one enzyme selected from a nucleic acid selected from:
(a) SEQ.ID NO: 4, SEQ.ID NO: 6, and SEQ.ID NO: 11;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 5, SEQ.ID NO: 7 or SEQ.ID NO: 12;
(f) a nucleic acid sequence that encodes a functional variant of any one of the amino acid sequences set forth in SEQ.ID NO: 5, SEQ.ID NO:7 or SEQ.ID NO: 12; and
(g) a nucleic acid sequence that hybridizes under stringent conditions to any one of the nucleic acid sequences set forth in (a), (b), (c), (d), (e) or (f).

In at least one embodiment, in an aspect, the nitrated psilocybin precursor compound can be a compound, having the formula (XXIV):

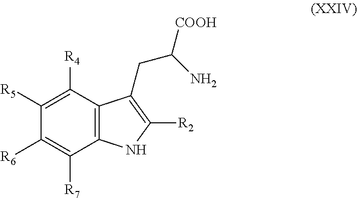

wherein at least one of R₂, R₄, R₅, R₆ and R₇ is a nitro group, wherein R₂, R₄, R₅, R₆ and R₇ when they are not nitrated are hydrogen atoms, or an alkyl, O-alkyl or O-aryl group, wherein R₄ when it is not nitrated is a hydrogen atom, an alkyl, O-alkyl or O-aryl group, a hydroxy group, or a phosphate group;
   wherein the psilocybin biosynthetic enzyme complement can comprise: a tryptophan decarboxylase encoded by a nucleic acid sequence selected from:
(a) SEQ.ID NO: 11;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having any one of the amino acid sequences set forth in SEQ.ID NO: 12;
(f) a nucleic acid sequence that encodes a functional variant of the amino acid sequence set forth in SEQ.ID NO: 12; and
(g) a nucleic acid sequence that hybridizes under stringent conditions to any one of the nucleic acid sequences set forth in (a), (b), (c), (d), (e) or (f); and the formed nitrated psilocybin derivative can be a compound having formula (XXV):

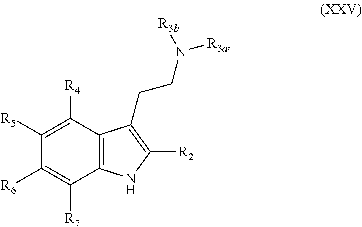

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein at least one of R_{3A} and R_{3B} are hydrogen atom.

In at least one embodiment, in an aspect, the nitrated psilocybin precursor compound can be a nitrated indole compound having the formula (XXVI):

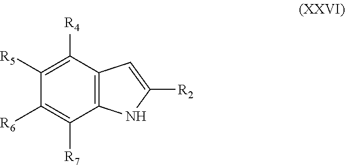

wherein at least one of R₂, R₄, R₅, R₆ and R₇ is a nitro group, wherein R₂, R₄, R₅, R₆ and R₇ when they are not nitrated are hydrogen atoms, or an alkyl, O-alkyl or O-aryl group, wherein R₄ when it is not nitrated is a hydrogen atom, an alkyl, O-alkyl or O-aryl group, a hydroxy group, or a phosphate group;
   wherein the psilocybin biosynthetic enzyme complement can comprise:

(i) a tryptophan synthase subunit B polypeptide encoded by a nucleic acid selected from:
(a) SEQ.ID NO: 6;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having any one of the amino acid sequences set forth in SEQ.ID NO: 7;
(f) a nucleic acid sequence that encodes a functional variant of the amino acid sequence set forth in SEQ.ID NO: 7; and
(g) a nucleic acid sequence that hybridizes under stringent conditions to any one of the nucleic acid sequences set forth in (a), (b), (c), (d), (e) or (f); and

(ii) a tryptophan decarboxylase encoded by a nucleic acid sequence selected from:
(a) SEQ.ID NO: 11;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having any one of the amino acid sequences set forth in SEQ.ID NO: 12;
(f) a nucleic acid sequence that encodes a functional variant of the amino acid sequence set forth in SEQ.ID NO: 12; and
(g) a nucleic acid sequence that hybridizes under stringent conditions to

any one of the nucleic acid sequences set forth in (a), (b), (c), (d), (e) or (f); and wherein the formed nitrated psilocybin derivative can be a compound having formula (XXV):

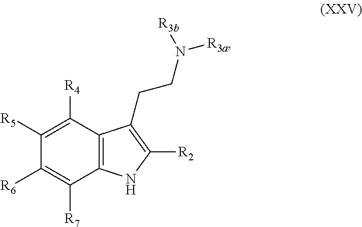

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein at least one of R_{3A} and R_{3B} are hydrogen atom.

In at least one embodiment, in an aspect, R_{3A} and R_{3B} in formula (XXV) are each a hydrogen atom.

In at least one embodiment, in an aspect, the psilocybin biosynthetic enzyme complement can further comprise an N-acetyl transferase.

In at least one embodiment, in an aspect, the N-acetyl transferase can be an enzyme encoded by. a nucleic acid sequence selected from:
(a) SEQ.ID NO: 4;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having any one of the amino acid sequences set forth in SEQ.ID NO: 5;
(f) a nucleic acid sequence that encodes a functional variant of the amino acid sequence set forth in SEQ.ID NO: 5; and
(g) a nucleic acid sequence that hybridizes under stringent conditions to any one of the nucleic acid sequences set forth in (a), (b), (c), (d), (e) or (f).

In at least one embodiment, in an aspect, the formed nitrogenated psilocybin compound can have the formula (XXVII):

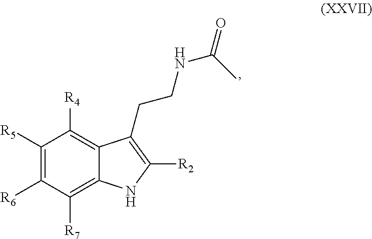

wherein, at least one of R₂, R₄, R₅, R₆ or R₇ is a nitro group, wherein each non-nitrated R₂, R₅, R₆, or R₇ is a hydrogen atom or an alkyl group or O-alkyl group, wherein R₄ when it is not nitrated is a phosphate group, a hydrogen atom or an alkyl group or O-alkyl group.

In at least one embodiment, in an aspect, the nitrated psilocybin derivative compound having formula (I) can be selected from the group consisting of compounds having formulas (III); (IV); (V); (VI); (VII); (VIII); (IX); (X); (XXVIII); and (XXIX):

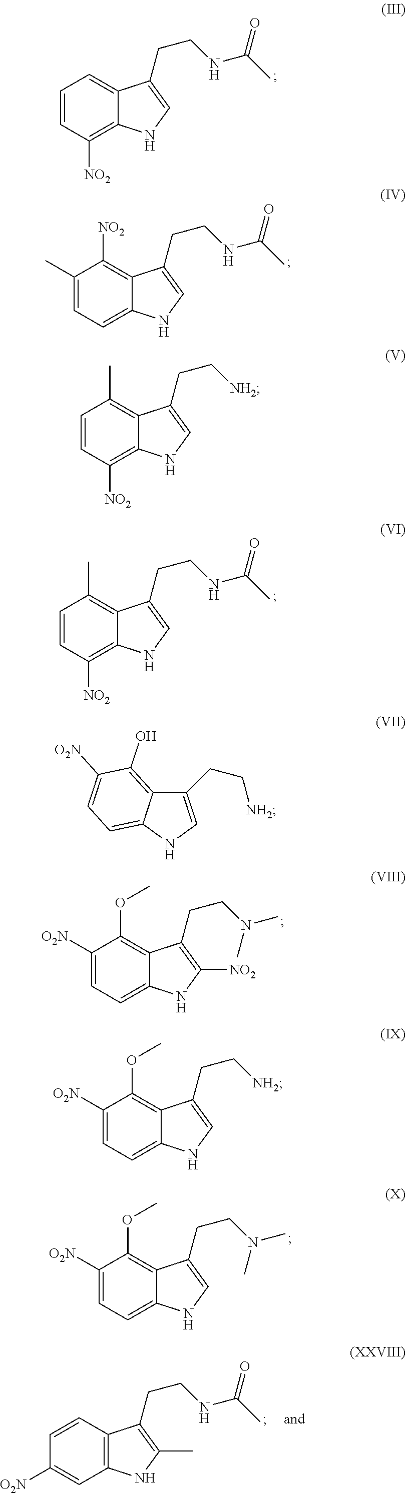

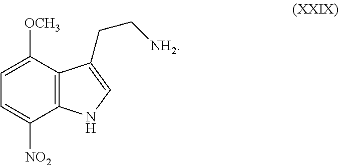 [SMILES: COc1ccc([N+](=O)[O-])c2[nH]cc(CCN)c12]

In at least one embodiment, in an aspect, the nitrated psilocybin precursor compound can be contacted with the host cell by including the nitrated psilocybin precursor compound in a growth medium for the host cell.

In at least one embodiment, in an aspect, the method can further include a step comprising isolating the nitrated psilocybin derivative.

In at least one embodiment, in an aspect, the host cell can be a microbial cell.

In at least one embodiment, in an aspect, the host cell can be a bacterial cell or a yeast cell.

In another aspect, the present disclosure provides, in at least one embodiment, a method for modulating a 5-HT_{2A} receptor, the method comprising contacting a 5-HT_{2A} receptor with a chemical compound or salt thereof having formula (I):

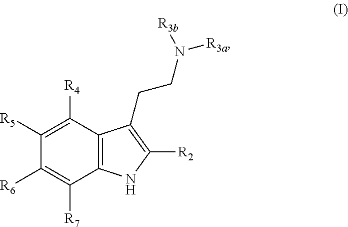

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group or a phosphate group, and wherein Ra and R_{3B} are a hydrogen atom, an alkyl group, an aryl group, or an acyl group under reaction conditions sufficient to thereby modulate receptor activity.

In some embodiments, in an aspect, the reaction conditions can be in vitro reaction conditions.

In some embodiments, in an aspect, the reaction conditions can be in vivo reaction conditions.

In another aspect, the present disclosure provides, in at least one embodiment, a use of a chemical compound having the formula (I):

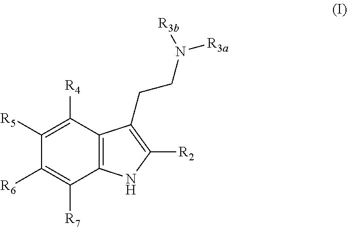

wherein, at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a phosphate group, a hydrogen atom, or a hydroxy group, an alkyl, O-alkyl or O-aryl group and wherein R_{3A} and R_{3B} are a hydrogen atom, an alkyl, O-alkyl or O-aryl group, in the manufacture of a pharmaceutical or recreational drug formulation.

In at least one embodiment, in an aspect, the manufacture can comprise formulating the chemical compound with an excipient, diluent or carrier.

In at least one embodiment, in an aspect, the manufacture can further include a step comprising derivatizing the chemical compound having the formula (I) by substituting the nitro group with another group or an atom.

In another aspect, the present disclosure provides, in at least one embodiment, a use of a chemical compound having the formula (I):

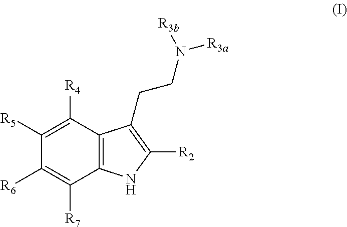

wherein, at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a phosphate group, a hydrogen atom, a hydroxy group, or an alkyl, O-alkyl or O-aryl group, and wherein R_{3A} and R_{3B} are a hydrogen atom, an alkyl, O-alkyl or O-aryl group, together with a diluent, carrier, or excipient as a pharmaceutical or recreational drug formulation.

Other features and advantages will become apparent from the following detailed description. It should be understood, however, that the detailed description, while indicating preferred implementations of the disclosure, are given by way of illustration only, since various changes and modifications within the spirit and scope of the disclosure will become apparent to those of skill in the art from the detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more dearly how the various embodiments may be carried into effect. The figures are not intended to limit the present disclosure.

FIG. 1 depicts the chemical structure of psilocybin.

FIG. 2 depicts a certain prototype structure of psilocybin and psilocybin derivative compounds, namely an indole. Certain carbon and nitrogen atoms may be referred to herein by reference to their position within the indole structure, i.e. N₁, C₂, C₃ etc. The pertinent atom numbering is shown.

FIGS. 3A, 3B, 3C, 3D, 3E, 3F, 3G, 3H, 3I, 3J, 3K, 3L, 3M, 3N, 3O, 3P and 3Q depict the chemical structures of certain example nitrated psilocybin derivative compounds, notably a 2-nitro psilocybin derivative (FIG. 3A); a 4-nitro derivative (FIG. 3B); a 5-nitro psilocybin derivative (FIG. 3C); a 6-nitro psilocybin derivative (FIG. 3D); a 7-nitro psilocybin derivative (FIG. 3E); a 2-nitro-4-phospho psilocybin derivative (FIG. 3F); a 4-phospho-5-nitro psilocybin derivative (FIG. 3G); a 4-phospho-6-nitro psilocybin derivative (FIG. 3H); a 4-phospho-7-nitro psilocybin derivative (FIG. 3I); a 2-nitro-4-methyl psilocybin derivative (FIG. 3J); 4-ethyl-5-nitro psilocybin derivative (FIG. 3K); a 2-methyl-6-nitro psilocybin derivative (FIG. 3L); a 4-propyl-7-nitro psilocybin derivative (FIG. 3M); a 2-nitro-4-O-methyl psilocybin derivative (FIG. 3N); 4-O-ethyl-5-nitro psilocybin derivative (FIG. 3O); a 4-O-methyl-6-nitro psilocybin derivative (FIG. 3P); a 4-O-propyl-7-nitro psilocybin derivative (FIG. 3Q). It is noted that in each of FIGS. 3A, 3B, 3C, 3D, 3E, 3F, 3G, 3H, 3I, 3J, 3K, 3L, 3M, 3N, 3O, 3P and 3Q R₃ₐ and R_{3b} can be a hydrogen atom, an alkyl group, an aryl group, or an acyl group.

FIGS. 4A, 4B, 4C, 4D, 4E, 4F, 4G, 4H, 4I and 4J depict the chemical structures of certain further example nitrated psilocybin derivative compounds, notably a 2,4-di-nitro psilocybin derivative (FIG. 4A); a 2,5-nitro psilocybin derivative (FIG. 4B); a 2,6-di-nitro-4-methyl psilocybin derivative (FIG. 4C); a 2,7-di-nitro-4-phospho psilocybin derivative (FIG. 4D); a 4,5-di-nitro psilocybin derivative (FIG. 4E); a 4,6-di-nitro psilocybin derivative (FIG. 4F); a 4,7-di-nitro psilocybin derivative (FIG. 4G); a 4-phospho-5,6-di-nitro psilocybin derivative (FIG. 4H) a 4-phospho-5,7-di-nitro psilocybin derivative (FIG. 4I); and a 6,7-di-nitro psilocybin derivative (FIG. 4J). It is noted that in each of FIGS. 4A, 48, 4C, 4D, 4E, 4F, 4G, 4H, 4I and 4J R₃ₐ and R_{3b} can be a hydrogen atom, an alkyl group, an aryl group, or an acyl group.

FIGS. 5A, 5B, 5C, 5D, 5E, and 5F depict the chemical structures of certain further example nitrated psilocybin derivative compounds, notably a 2,4,5-tri-nitro psilocybin derivative (FIG. 5A); a 2-5,6-tri-nitro-4-methyl psilocybin derivative (FIG. 5B); a 2,5,7-tri-nitro psilocybin derivative (FIG. 5C); a 4,5,6-tri-nitro psilocybin derivative (FIG. 5D); a 4,5,7-ti-nitro psilocybin derivative (FIG. 5E); and a 4-phospho-5,6,7-tri-nitro psilocybin derivative (FIG. 5F). It is noted that in each of FIGS. 5A, 5B, 5C, 5D, 5E, and 5F R₃ₐ and R_{3b} can be a hydrogen atom, an alkyl group, an aryl group, or an acyl group.

FIGS. 6A, 6B, 6C, 6D, and 6E depict the chemical structures of certain further example nitrated psilocybin derivative compounds, notably a 2,4,5,6-tetra-nitro psilocybin derivative (FIG. 6A); a 4,5,6,7-tetra-nitro psilocybin derivative (FIG. 6B); a 2,5,6-7-tetra-nitro-4-phospho psilocybin derivative (FIG. 6C); a 2,4,6,7-tetra hydroxy psilocybin derivative (FIG. 6D); and a 2,4,5,7-tetra-nitro psilocybin derivative (FIG. 6E). It is noted that in each of FIGS. 6A, 6B, 6C, 6D, and 6E R₃ₐ and R_{3b} can be a hydrogen atom, an alkyl group, an aryl group, or an acyl group.

FIGS. 7A, 7B, 7C, 7D, 7E, and 7F depict the chemical structures of certain example reactant psilocybin derivatives, notably a 4-O-methyl-psilocybin derivative (FIG. 7A), a 4-O-ethyl-psilocybin derivative (FIG. 7B), a 4-methyl-psilocybin derivative (FIG. 8C), a 4-ethyl-psilocybin derivative (FIG. 7D), a 4-hydroxy-psilocybin derivative (FIG. 7E), and a 4-phospho-psilocybin derivative (FIG. 7F). It is noted that in each of FIGS. 7A, 7B, 7C, 7D, 7E, and 7F R₃ₐ and R_{3b} can be a hydrogen atom, an alkyl group, an aryl group, or an acyl group.

FIG. 8 depicts an example chemical reaction for synthesizing a nitrated psilocybin derivative, notably a reaction wherein a 4-O-methyl psilocybin derivative is reacted with nitric acid in the presence of sulfuric acid to form a 4-O-methyl-5-nitro psilocybin derivative.

FIGS. 9A and 9B depict example chemical synthesis processes for the synthesis of certain example nitrated psilocybin derivatives, notably an example process for synthesis of an example nitrated psilocybin derivative (denoted as compounds 9A-7) (FIG. 9A) and an example process for example nitrated psilocybin derivatives (denoted as compounds 9B-9, 9B-9, 9B-10, 9B-11, 9B-12 and 9B-13) (FIG. 9B).

FIG. 10 depicts an example biosynthesis process for the synthesis of a nitrated psilocybin derivative.

FIG. 11 depicts a graph obtained in the performance of an experimental assay to evaluate the efficacy of an example nitrate psilocybin derivative, notably a cell viability assay involving an example nitrated psilocybin derivative compound having the chemical formula (XXIX) set forth herein.

FIGS. 12A, 12B, 12C, 12D, 12E, and 12F depict graphs obtained in the performance of an experimental assay to evaluate the efficacy of an example nitrate psilocybin derivative, notably a 5-HT₂ₐ receptor modulation, in particular a calcium flux assay involving psilocin (positive control) and +5-HT₂ₐ cells (FIG. 12A), serotonin (positive control) and +5-HT₂ₐ cells (FIG. 12B), mexamine (positive control) and +5-HT₂ₐ cells (FIG. 12C), a nitrated psilocybin derivative having chemical formula (XXIX) and +5-HT₂ₐ cells (FIG. 12D), a nitrated psilocybin derivative having chemical formula (XXIX) and −5-HT₂ₐ cells (FIG. 12E), and methanol (negative control) and +5-HT₂ₐ cells (FIG. 12F).

FIGS. 13A and 13B depict a representation of mass spectrometry data in the form of a chromatogram, notably a chromatogram obtained in the performance of an experiment to synthesize an example nitrated psilocybin derivative compound having the chemical formula (III) set forth herein (FIG. 13A); and in the form of a mass spectrometry spectrum obtained in the performance of an experiment to identify a nitrated psilocybin derivative compound having the chemical formula (III) set forth herein (FIG. 13B).

FIGS. 14A and 14B depict a representation of mass spectrometry data in the form of a chromatogram, notably a chromatogram obtained in the performance of an experiment to synthesize an example nitrated psilocybin derivative compound having the chemical formula (IV) set forth herein (FIG. 14A); and in the form of a mass spectrometry spectrum obtained in the performance of an experiment to identify a nitrated psilocybin derivative compound having the chemical formula (IV) set forth herein (FIG. 14B).

FIGS. 16A and 15B depict a representation of mass spectrometry data in the form of a chromatogram, notably a chromatogram obtained in the performance of an experiment to synthesize an example nitrated psilocybin derivative compound having the chemical formula (V) set forth herein (FIG. 15A); and in the form of a mass spectrometry spectrum obtained in the performance of an experiment to identify a nitrated psilocybin derivative compound having the chemical formula (V) set forth herein (FIG. 15B).

FIG. 16 depicts a representation of mass spectrometry data in the form of a chromatogram, notably a chromatogram obtained in the performance of an experiment to synthesize an example nitrated psilocybin derivative compound having the chemical formula (VI) set forth herein.

FIGS. 17A and 17B depict a representation of mass spectrometry data in the form of a chromatogram, notably a chromatogram obtained in the performance of an experiment to synthesize an example nitrated psilocybin derivative compound having the chemical formula (XXIX) set forth herein (FIG. 17A) and in the form of a mass spectrometry spectrum obtained in the performance of an experiment to identify a nitrated psilocybin derivative compound having the chemical formula (XXIX) set forth herein (FIG. 17B).

The figures together with the following detailed description make apparent to those skilled in the art how the disclosure may be implemented in practice.

### DETAILED DESCRIPTION

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

As used herein and in the claims, the singular forms, such “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers.

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

When ranges are used herein for physical properties, such as molecular weight, or chemical properties, such as chemical formulae, all combinations and sub-combinations of ranges and specific embodiments therein are intended to be included. Other than in the operating examples, or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Furthermore any range of values described herein is intended to specifically include the limiting values of the range, and any intermediate value or sub-range within the given range, and all such intermediate values and sub-ranges are individually and specifically disclosed (e.g. a range of 1 to 5 includes 1, 1.5, 2, 2.75, 3, 3.90, 4, and 5). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only, and is not intended to limit the scope of the present invention, which is defined solely by the claims.

All publications, patents and patent applications are herein incorporated by reference in their entirety to the same extent as if each individual publication, patent or patent application was specifically and individually indicated to be incorporated by reference in its entirety.

### Terms and Definitions

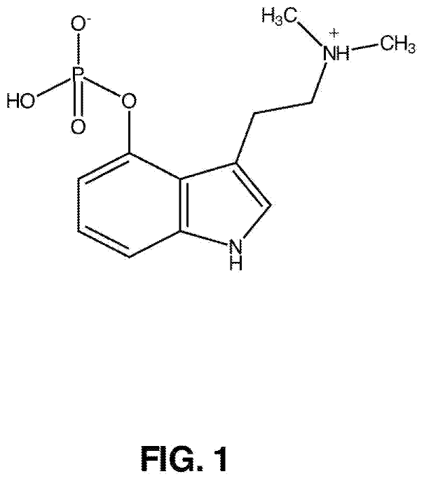

The term “psilocybin”, refers to a chemical compound having the structure set forth in FIG. 1.

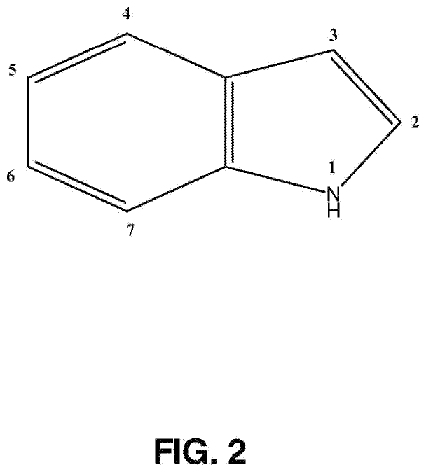

The term “indole prototype structure” refers to the chemical structure shown in FIG. 2. It is noted that specific carbon atoms and a nitrogen atom in the indole prototype structure are numbered. Reference may be made to these carbon and nitrogen numbers herein, for example C₂, C₄, N₁, and so forth. Furthermore, reference may be made to chemical groups attached to the indole prototype structure in accordance with the same numbering, for example R₄ and R₆ reference chemical groups attached to the C₄ and C₆ atom, respectively. In addition, R_{3A} and R_{3B}, in this respect, reference chemical groups extending from the 2-aminoethyl group extending in turn from the C₃ atom of the prototype indole structure.

The terms “nitrated psilocybin derivative” or “nitrated psilocybin derivative compound”, as used herein, refer to a psilocybin derivative compound comprising one or more nitro groups. Reference may be made to specific carbon atoms which may be nitrated. For example, a 7-nitro-psilocybin derivative refers to a nitrated psilocybin derivative in which carbon atom number 7 (as identified in the indole prototype structure) is nitrated, or, similarly, 2-nitro-psilocybin derivative refers to a nitrated psilocybin derivative in which carbon atom number 2 (as identified in the indole prototype structure) is nitrated. Thus, for example, nitrated psilocybin derivatives include, single nitro derivatives, 2-nitro, 4-nitro, 5-nitro, 6-nitro and 7-nitro psilocybin derivatives, for example, and multiple nitro derivatives, such as, for example, 4,7-di-nitro-psilocybin derivatives, 2,5,7-tri-nitro-psilocybin derivatives etc. The term nitrated psilocybin derivatives further includes chemical compounds having the chemical formula (I):

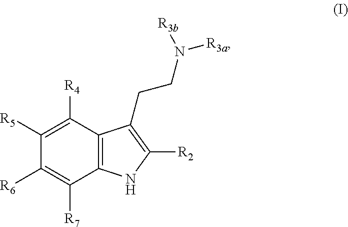

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen, an alkyl group, an aryl group, or an acyl group. The term further includes salts of nitrated psilocybin derivatives, such as a sodium salt, a potassium salt etc.

The terms “nitro” and “nitro group”, as used herein, refer to a molecule containing one atom of nitrogen bonded to two atoms of oxygen and having the formula —NO₂. A nitro group through its nitrogen atom may be chemically bonded to another entity. Furthermore, it is noted that an entity attached to a nitro group may be referred to herein as a “nitrated” entity, e.g. a nitrated psilocybin derivative is a psilocybin derivative possessing a nitro group.

The term “reactant psilocybin derivative”, as used herein, refers to any psilocybin derivative suitable for reaction with a nitro group donating compound, such as nitric acid (HNO₃), a nitrate salt, or an acyl nitrate, for example, to form a nitrated psilocybin derivative in such a manner that an existing atom or group of the reactant psilocybin derivative is substituted with a nitro group.

The term “nitrated indole compound”, as used herein refers to an indole comprising compound wherein at least one of the carbon atoms is nitrated and includes a compound having the formula (XXVI):

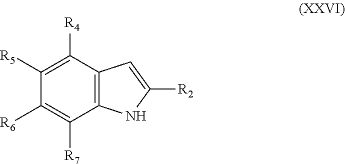

wherein at least one of R₂, R₄, R₅, R₆ and R₇ is a nitro group, wherein R₂, R₄, R₅, R₆ and R₇ when they are not nitrated are hydrogen atoms, or an alkyl, O-alkyl or O-aryl group, wherein R₄ when it is not nitrated is a hydrogen atom, an alkyl, O-alkyl or O-aryl group, a hydroxy group, or a phosphate group.

The term “tryptophan”, as used herein, refers to a molecule having the chemical structure (XXIV):

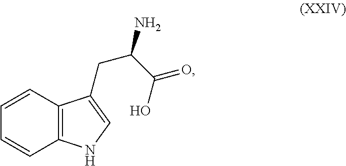 [SMILES: N[C@H](Cc1c[nH]c2ccccc12)C(=O)O]

and further includes its D-enantiomeric form (not shown).

The term “psilocybin precursor compound”, as used herein, refers to a chemical compound that may serve as a precursor compound in the synthesis or biosynthesis of a psilocybin derivative, including, notably, the synthesis or biosynthesis of a nitrated psilocybin derivative, and includes compounds comprising an indole prototype structure, including, for example, indole or tryptophan, and further includes nitrated derivatives and salts of any of the foregoing, such as, for example a nitrated indole or a nitrated tryptophan.

The term “nitrated psilocybin precursor compound”, as used herein, refers to a psilocybin precursor compound possessing a nitro group. Reference may be made to specific carbon atoms of the psilocybin precursor compound which may be nitrated, for example, 6-nitro-indole refers to a nitrated indole in which carbon atom number 7 (as identified in the indole prototype structure) is nitrated, or, similarly, 6-nitro-trptophan refers to a tryptophan in which carbon atom number 6 (as identified in the indole prototype structure) is nitrated.

The term “phosphate group”, as used herein, is a molecule containing one atom of phosphorus, covalently bound to four oxygen atoms (three single bonds and one double bond). Of the four oxygen atoms, one oxygen atom may be a hydroxy, and one of the non-hydroxylated oxygen atom may be chemically bonded to another entity.

The terms “hydroxy group”, and “hydroxy”, as used herein, refer to a molecule containing one atom of oxygen bonded to one atom of hydrogen, and having the formula —OH. A hydroxy through its oxygen atom may be chemically bonded to another entity.

The term “alkyl”, as used herein, refers to a straight and/or branched chain, saturated alkyl radical containing from one to “p” carbon atoms (“C₁-Cₚ-alkyl”) and includes, depending on the identity of “p”, methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable p is an integer representing the largest number of carbon atoms in the alkyl radical. Alkyl groups further include hydrocarbon groups arranged in a chain having the chemical formula —CₙH₂ₙ₊₁, including, without limitation, methyl groups (—CH₃), ethyl groups (—C₂H₅), propyl groups (—C₃H₇), and butyl groups (—C₄H₉).

The term “O-alkyl”, as used herein, refers to a hydrocarbon group (an alkyl group as defined herein) arranged in a chain having the chemical formula —O—CₙH₂ₙ₊₁. O-alkyl groups include, without limitation, O-methyl groups (—O—CH₃), O-ethyl groups (—O—C₂H₅), O-propyl groups (—O—C₃H₇) and O-butyl groups (—O—C₄H₉).

The term “aryl”, as used herein, refers to a monocyclic, bicyclic or tricyclic aromatic ring system containing, depending on the number of atoms in the rings, for example, from 6 to 14 carbon atoms (C₆-C₁₄-aryl) or from 6 to 10 carbons (C₆-C₁₀-aryl), and at least 1 aromatic ring and includes phenyl, naphthyl, anthracenyl, 1,2-dihydronaphthyl, 1,2,3,4-tetrahydronaphthyl, fluorenyl, phenanthrenyl, biphenylenyl, indanyl, indenyl and the like.

The term “O-aryl group”, as used herein, refers to an aryl group in which the carbon atom in the aromatic ring is single bonded to an additional oxygen atom. The additional oxygen atom can be bonded to another entity.

The term “acyl”, as used herein, refers to a carbon atom double bonded to an oxygen and single bonded to an alkyl group (as defined herein). The carbon atom further can be bonded to another entity. An acyl group can be described by the chemical formula: —C(═O)—CₙH₂ₙ₊₁.

The term “5-HT_{2A} receptor”, as used herein, refers to a subclass of a family of receptors for the neurotransmitter and peripheral signal mediator serotonin. 5-HT_{2A} receptors can mediate a plurality of central and peripheral physiologic functions of serotonin. Central nervous system effects can include mediation of hallucinogenic effects of hallucinogenic compounds.

The term “modulating 5-HT_{2A} receptors”, as used herein, refers to the ability of a compound disclosed herein to alter the function of 5-HT_{2A} receptors. A 5-HT_{2A} receptor modulator may activate the activity of a 5-HT_{2A} receptor, may activate or inhibit the activity of a 5-HT_{2A} receptor depending on the concentration of the compound exposed to the 5-HT_{2A} receptor, or may inhibit the activity of a 5-HT_{2A} receptor. Such activation or inhibition may be contingent on the occurrence of a specific event, such as activation of a signal transduction pathway, and/or maybe manifest only in particular cell types. The term “modulating 5-HT_{2A} receptors,” also refers to altering the function of a 5-HT_{2A} receptor by increasing or decreasing the probability that a complex forms between a 5-HT_{2A} receptor and a natural binding partner to form a multimer. A 5-HT_{2A} receptor modulator may increase the probability that such a complex forms between the 5-HT_{2A} receptor and the natural binding partner, may increase or decrease the probability that a complex forms between the 5-HT_{2A} receptor and the natural binding partner depending on the concentration of the compound exposed to the 5-HT_{2A} receptor, and or may decrease the probability that a complex forms between the 5-HT_{2A} receptor and the natural binding partner. Furthermore, the term includes allosteric modulation of the receptor 5-HT_{2A}, i.e. modulation of the 5-HT_{2A} receptor through interaction with the 5-HT_{2A} receptor that is topographically different than the orthosteric site recognized by the cell's endogenous agonist, such modulation further including positive allosteric modulation (PAM), negative allosteric modulation (NAM) and silent allosteric modulation (SAM).

The term “5-HT_{2A} receptor-mediated disorder”, as used herein, refers to a disorder that is characterized by abnormal 5-HT_{2A} receptor activity. A 5-HT_{2A} receptor-mediated disorder may be completely or partially mediated by modulating 5-HT_{2A} receptors. In particular, a 5-HT_{2A} receptor-mediated disorder is one in which modulation of 5-HT_{2A} receptors results in some effect on the underlying disorder e.g., administration of a 5-HT_{2A} receptor modulator results in some improvement in at least some of the subjects being treated.

The term “pharmaceutical formulation”, as used herein, refers to a preparation in a form which allows an active ingredient, including a psychoactive ingredient, contained therein to provide effective treatment, and which does not contain any other ingredients which cause excessive toxicity, an allergic response, irritation, or other adverse response commensurate with a reasonable risk/benefit ratio. The pharmaceutical formulation may contain other pharmaceutical ingredients such as excipients, carriers, diluents, or auxiliary agents.

The term “recreational drug formulation”, as used herein, refers to a preparation in a form which allows a psychoactive ingredient contained therein to be effective for administration as a recreational drug, and which does not contain any other ingredients which cause excessive toxicity, an allergic response, irritation, or other adverse response commensurate with a reasonable risk/benefit ratio. The recreational drug formulation may contain other ingredients such as excipients, carriers, diluents, or auxiliary agents.

The term “effective for administration as a recreational drug”, as used herein, refers to a preparation in a form which allows a subject to voluntarily induce a psychoactive effect for non-medical purposes upon administration, generally in the form of self-administration. The effect may include an altered state of consciousness, satisfaction, pleasure, euphoria, perceptual distortion, or hallucination.

The term “effective amount”, as used herein, refers to an amount of an active agent, pharmaceutical formulation or recreational drug formulation, sufficient to induce a desired biological or therapeutic effect, including a prophylactic effect, and further including a psychoactive effect. Such effect can include an effect with respect to the signs, symptoms or causes of a disorder, or disease or any other desired alteration of a biological system. The effective amount can vary depending, for example, on the health condition, injury stage, disorder stage, or disease stage, weight, or sex of a subject being treated, timing of the administration, manner of the administration, age of the subject, and the like, all of which can be determined by those of skill in the art.

The terms “treating” and “treatment”, and the like, as used herein, are intended to mean obtaining a desirable physiological, pharmacological, or biological effect, and includes prophylactic and therapeutic treatment. The effect may result in the inhibition, attenuation, amelioration, or reversal of a sign, symptom or cause of a disorder, or disease, attributable to the disorder, or disease, which includes mental and psychiatric diseases and disorders. Clinical evidence of the prevention or treatment may vary with the disorder, or disease, the subject and the selected treatment.

The term “pharmaceutically acceptable”, as used herein, refers to materials, including excipients, carriers, diluents, or auxiliary agents, that are compatible with other materials in a pharmaceutical or recreational drug formulation and within the scope of reasonable medical judgement suitable for use in contact with a subject without excessive toxicity, allergic response, irritation, or other adverse response commensurate with a reasonable risk/benefit ratio.

The term “psilocybin biosynthetic enzyme complement”, as used herein, refers to one or more polypeptides which alone or together are capable of facilitating the chemical conversion of a psilocybin precursor compound and form another psilocybin precursor compound, or a nitrated psilocybin derivative compound. A psilocybin biosynthetic enzyme complement can include, for example, a tryptophan synthase subunit B polypeptide, a tryptophan decarboxylase and/or a N-acetyl transferase.

The term “tryptophan synthase subunit B polypeptide” as used herein, refers to any and all enzymes comprising a sequence of amino acid residues which is (i) substantially identical to the amino acid sequences constituting any tryptophan synthase subunit B polypeptide set forth herein, including, for example, SEQ.ID NO: 7, or (ii) encoded by a nucleic acid sequence capable of hybridizing under at least moderately stringent conditions to any nucleic acid sequence encoding any tryptophan synthase subunit B polypeptide set forth herein, but for the use of synonymous codons.

The term “tryptophan decarboxylase” as used herein, refers to any and all enzymes comprising a sequence of amino acid residues which is (i) substantially identical to the amino acid sequences constituting any tryptophan decarboxylase polypeptide set forth herein, including, for example, SEQ.ID NO: 12, or (ii) encoded by a nucleic acid sequence capable of hybridizing under at least moderately stringent conditions to any nucleic acid sequence encoding any tryptophan decarboxylase set forth herein, but for the use of synonymous codons.

The term “N-acetyl transferase” as used herein, refers to any and all enzymes comprising a sequence of amino acid residues which is (i) substantially identical to the amino acid sequences constituting any N-acetyl transferase polypeptide set forth herein, including, for example, SEQ.ID NO: 5, or (ii) encoded by a nucleic acid sequence capable of hybridizing under at least moderately stringent conditions to any nucleic acid sequence encoding any N-acetyl transferase set forth herein, but for the use of synonymous codons.

The terms “nucleic acid sequence encoding tryptophan synthase subunit B polypeptide”, as may be used interchangeably herein, refer to any and all nucleic acid sequences encoding a tryptophan synthase subunit B polypeptide, including, for example, SEQ.ID NO: 6. Nucleic acid sequences encoding a tryptophan synthase subunit B polypeptide further include any and all nucleic acid sequences which (i) encode polypeptides that are substantially identical to the tryptophan synthase subunit B polypeptide sequences set forth herein; or (ii) hybridize to any tryptophan synthase subunit B polypeptide nucleic acid sequences set forth herein under at least moderately stringent hybridization conditions or which would hybridize thereto under at least moderately stringent conditions but for the use of synonymous codons.

The terms “nucleic acid sequence encoding tryptophan decarboxylase”, and “nucleic acid sequence encoding a tryptophan decarboxylase polypeptide”, as may be used interchangeably herein, refer to any and all nucleic acid sequences encoding a tryptophan decarboxylase, including, for example, SEQ.ID NO: 11. Nucleic acid sequences encoding a tryptophan decarboxylase polypeptide further include any and all nucleic acid sequences which (i) encode polypeptides that are substantially identical to the tryptophan decarboxylase polypeptide sequences set forth herein; or (ii) hybridize to any tryptophan decarboxylase nucleic acid sequences set forth herein under at least moderately stringent hybridization conditions or which would hybridize thereto under at least moderately stringent conditions but for the use of synonymous codons.

The terms “nucleic acid sequence encoding N-acetyl transferase”, and “nucleic acid sequence encoding an N-acetyl transferase polypeptide”, as may be used interchangeably herein, refer to any and all nucleic acid sequences encoding an N-acetyl transferase, including, for example, SEQ.ID NO: 4. Nucleic acid sequences encoding an N-acetyl transferase polypeptide further include any and all nucleic acid sequences which (i) encode polypeptides that are substantially identical to the N-acetyl transferase polypeptide sequences set forth herein; or (ii) hybridize to any N-acetyl transferase nucleic acid sequences set forth herein under at least moderately stringent hybridization conditions or which would hybridize thereto under at least moderately stringent conditions but for the use of synonymous codons.

The terms “nucleic acid”, or “nucleic acid sequence”, as used herein, refer to a sequence of nucleoside or nucleotide monomers, consisting of naturally occurring bases, sugars and intersugar (backbone) linkages. The term also includes modified or substituted sequences comprising non-naturally occurring monomers or portions thereof. The nucleic acids of the present disclosure may be deoxyribonucleic nucleic acids (DNA) or ribonucleic acids (RNA) and may include naturally occurring bases including adenine, guanine, cytosine, thymidine and uracil. The nucleic acids may also contain modified bases. Examples of such modified bases include aza and deaza adenine, guanine, cytosine, thymidine and uracil, and xanthine and hypoxanthine. A sequence of nucleotide or nucleoside monomers may be referred to as a polynucleotide sequence, nucleic acid sequence, a nucleotide sequence or a nucleoside sequence.

The term “polypeptide”, as used herein in conjunction with a reference SEQ.ID NO, refers to any and all polypeptides comprising a sequence of amino acid residues which is (i) substantially identical to the amino acid sequence constituting the polypeptide having such reference SEQ.ID NO, or (ii) encoded by a nucleic acid sequence capable of hybridizing under at least moderately stringent conditions to any nucleic acid sequence encoding the polypeptide having such reference SEQ.ID NO, but for the use of synonymous codons. A sequence of amino acid residues may be referred to as an amino acid sequence, or polypeptide sequence.

The term “nucleic acid sequence encoding a polypeptide”, as used herein in conjunction with a reference SEQ.ID NO, refers to any and all nucleic acid sequences encoding a polypeptide having such reference SEQ.ID NO. Nucleic acid sequences encoding a polypeptide, in conjunction with a reference SEQ.ID NO, further include any and all nucleic acid sequences which (i) encode polypeptides that are substantially identical to the polypeptide having such reference SEQ.ID NO; or (ii) hybridize to any nucleic acid sequences encoding polypeptides having such reference SEQ.ID NO under at least moderately stringent hybridization conditions or which would hybridize thereto under at least moderately stringent conditions but for the use of synonymous codons.

By the term “substantially identical” it is meant that two amino acid sequences preferably are at least 70% identical, and more preferably are at least 85% identical and most preferably at least 95% identical, for example 96%, 97%, 98% or 99% identical. In order to determine the percentage of identity between two amino acid sequences the amino acid sequences of such two sequences are aligned, using for example the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Mol. Biol., 1990: 215:403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919 using a gap opening penalty of 10 and a gap extension penalty of 0.1, so that the highest order match obtained between two sequences wherein at least 50% of the total length of one of the two sequences is involved in the alignment.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.-16.6 (Log 10 [Na+])+0.41(% (G+C)−600/I), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm (based on the above equation) −5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

The term “functional variant”, as used herein in reference to polynucleotides or polypeptides, refers to polynucleotides or polypeptides capable of performing the same function as a noted reference polynucleotide or polypeptide. Thus, for example, a functional variant of the polypeptide set forth in SEQ.ID NO: 2, refers to a polypeptide capable of performing the same function as the polypeptide set forth in SEQ.ID NO: 2. Functional variants include modified a polypeptide wherein, relative to a noted reference polypeptide, the modification includes a substitution, deletion or addition of one or more amino acids. In some embodiments, substitutions are those that result in a replacement of one amino acid with an amino acid having similar characteristics. Such substitutions include, without limitation (i) glutamic acid and aspartic acid; (i) alanine, serine, and threonine; (iii) isoleucine, leucine and valine, (iv) asparagine and glutamine, and (v) tryptophan, tyrosine and phenylalanine. Functional variants further include polypeptides having retained or exhibiting an enhanced psilocybin biosynthetic bioactivity.

The term “chimeric”, as used herein in the context of nucleic acids, refers to at least two linked nucleic acids which are not naturally linked. Chimeric nucleic acids include linked nucleic acids of different natural origins. For example, a nucleic acid constituting a microbial promoter linked to a nucleic acid encoding a plant polypeptide is considered chimeric. Chimeric nucleic acids also may comprise nucleic acids of the same natural origin, provided they are not naturally linked. For example a nucleic acid constituting a promoter obtained from a particular cell-type may be linked to a nucleic acid encoding a polypeptide obtained from that same cell-type, but not normally linked to the nucleic acid constituting the promoter. Chimeric nucleic acids also include nucleic acids comprising any naturally occurring nucleic acids linked to any non-naturally occurring nucleic acids.

The terms “substantially pure” and “isolated”, as may be used interchangeably herein describe a compound, e.g., a secondary metabolite, psilocybin or a psilocybin derivative, polynucleotide or a polypeptide, which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., in the case of polypeptides, by chromatography, gel electrophoresis or HPLC analysis.

The term “recovered” as used herein in association with a chemical compound, refers to a more or less pure form of the chemical compound.

### General Implementation

As hereinbefore mentioned, the present disclosure relates to psilocybin derivatives. In particular, the present disclosure provides novel nitrated psilocybin derivatives. In general, the herein provided compositions exhibit functional properties which deviate from the functional properties of psilocybin. Thus, for example, the nitrated psilocybin derivatives, can exhibit pharmacological properties which deviate from psilocybin. Furthermore, the nitrated derivatives may psilocybin derivatives may exhibit physico-chemical properties which differ from psilocybin. Thus, for example, nitrated psilocybin derivatives may exhibit superior solubility in a solvent, for example, an aqueous solvent. The nitrated psilocybin derivatives in this respect are useful in the formulation of pharmaceutical and recreational drug formulations. Furthermore, the nitrated psilocybin compounds of the present disclosure may be used as a feedstock material for deriving further psilocybin derivatives. In one embodiment, the nitrated psilocybin derivatives of the present disclosure can conveniently be synthetically produced. The practice of this method avoids the extraction of psilocybin from mushrooms and the performance of subsequent chemical reactions to achieve nitrated derivatives. Furthermore, the growth of mushrooms can be avoided thus limiting the dependence on climate and weather, and potential legal and social challenges associated with the cultivation of mushrooms containing psychoactive compounds. The method can efficiently yield substantial quantities of nitrated psilocybin derivatives.

In what follows selected embodiments are described with reference to the drawings.

Initially example nitrated psilocybin derivatives will be described. Thereafter example methods of using and making the nitrated psilocybin derivatives will be described.

Accordingly, in one aspect, the present disclosure provides derivatives of a compound known as psilocybin of which the chemical structure is shown in FIG. 1. The derivatives herein provided are, in particular, derivatives of psilocybin including a nitro group.

Thus, in one aspect, the present disclosure provides, in accordance with the teachings herein, in at least one embodiment, a chemical compound or salt thereof having formula (I):

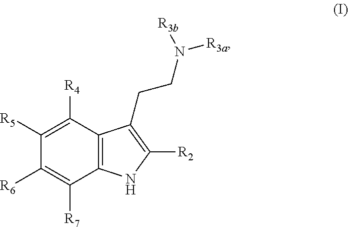

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen, an alkyl group, an aryl group or an acyl group.

Thus, referring to the chemical compound having formula (I), initially it is noted that, in an aspect thereof, at least one of R₂, R₄, R₅, R₆, or R₇ is a nitro group.

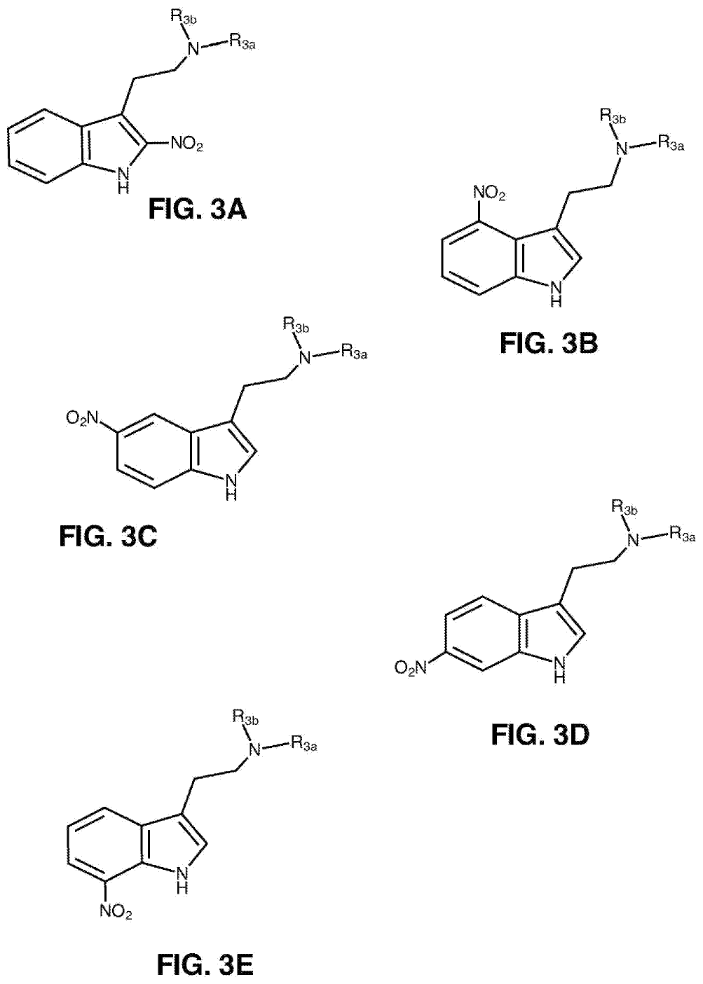

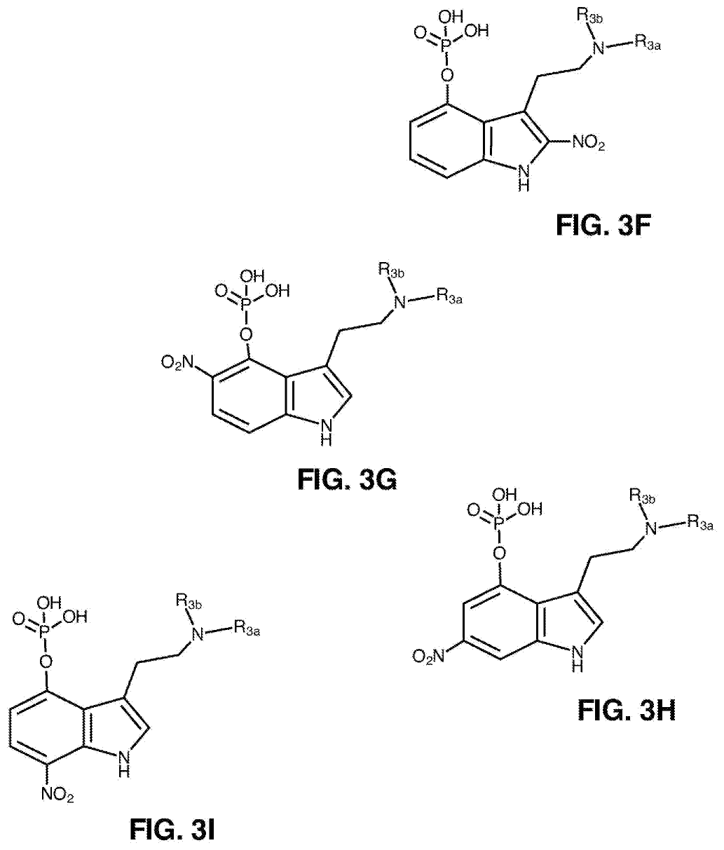

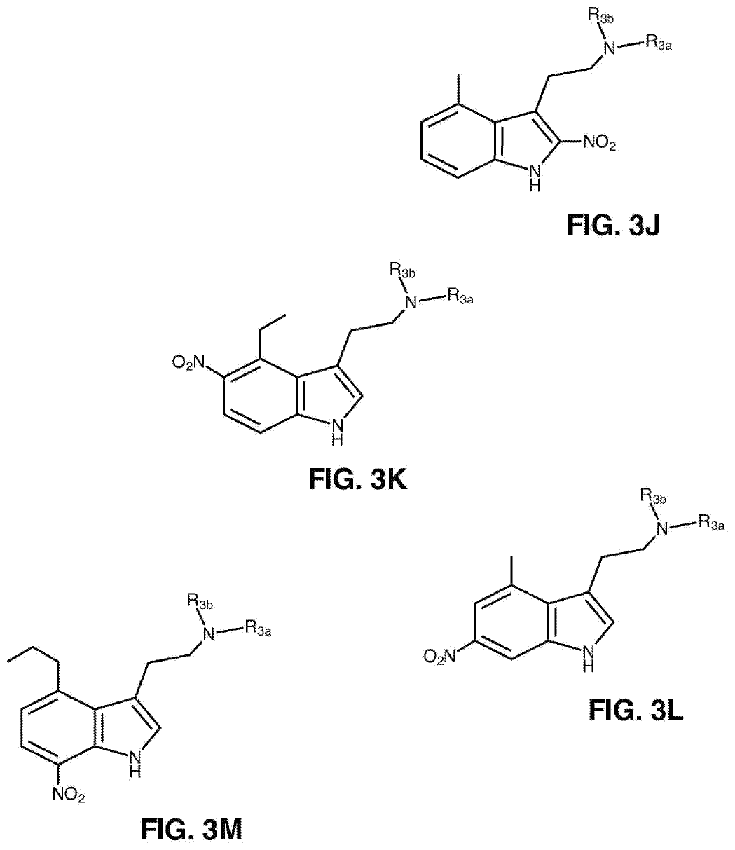

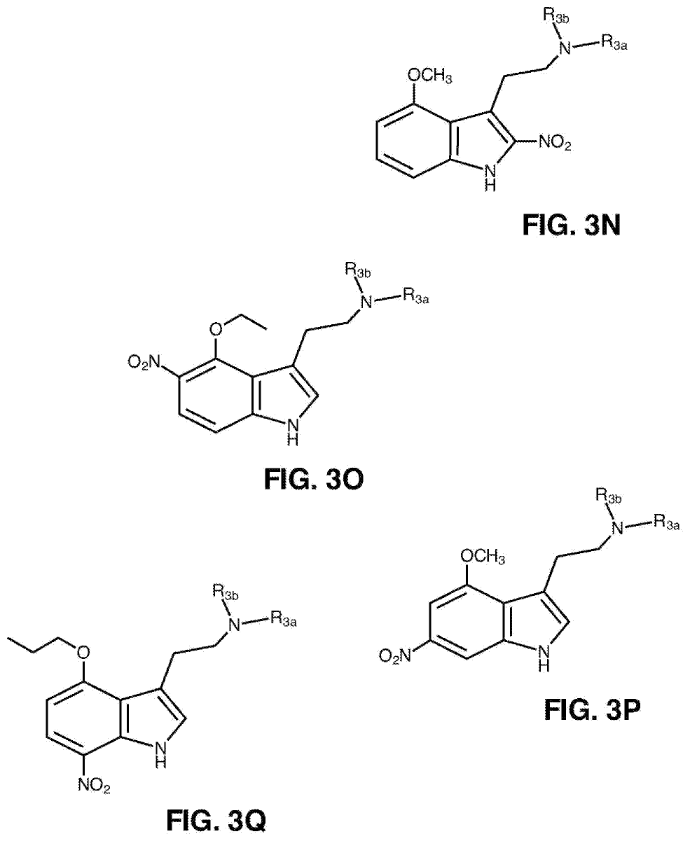

Continuing to refer to the chemical compound having formula (I), in one embodiment, one of R₂, R₄, R₅, R₆ and R₇ can be a nitro group. Thus, in one embodiment, R₂ can be a nitro group, each of R₅, R₆ and R₇ can be a hydrogen atom or an alkyl, O-alkyl or O-aryl group, and R₄ can be a phosphate group, a hydrogen atom, a hydroxy group, or an alkyl, O-alkyl or O-aryl group (see: the example nitrated psilocybin derivatives shown in FIG. 3A (R₂ is a nitro group; R₄ is a hydrogen atom; R₅, R₆ and R₇ are a hydrogen atom; R₃ₐ and R_{3b} are a hydrogen atom, an alkyl group, an aryl group, or an acyl group); FIG. 3F (R₂ is a nitro group; R₄ is a phosphate group; R₅, R₆ and R₇ are a hydrogen atom; R₃ₐ and R_{3b} are a hydrogen atom, an alkyl group, an aryl group, or an acyl group); FIG. 3J (R₂ is a nitro group; R₄ is a methyl group; R₅, R₆ and R₇ are a hydrogen atom; R₃ₐ and R_{3b} are a hydrogen atom, an alkyl group, an aryl group, or an acyl group); and FIG. 3N (R₂ is a nitro group; R₄ is an O-methyl group; R₅, R₆ and R₇ are a hydrogen atom; R₃ₐ and R_{3b} are a hydrogen atom, an alkyl group, an aryl group, or an acyl group)).

Continuing to refer to the chemical compound having formula (I), in one embodiment, R₄ can be a nitro group, and each of R₂, R₅, R₆ and R₇ can be a hydrogen atom or an alkyl, O-alkyl or O-aryl group (see: the example nitrated psilocybin derivative shown in FIG. 3B (R₄ is a nitro group; R₂, R₅, R₆ and R₇ are a hydrogen atom; and R₃ₐ and R_{3b} are a hydrogen atom, an alkyl group, an aryl group, or an acyl group)).

Continuing to refer to the chemical compound having formula (I), in one embodiment, R₅ can be a nitro group, and each of R₂, R₆ and R₇ can be a hydrogen atom or an alkyl, O-alkyl or O-aryl group, and R₄ can be a phosphate group, a hydrogen atom, a hydroxy group, or an alkyl, O-alkyl or O-aryl group (see: the example nitrated psilocybin derivatives shown in FIG. 3C (R₅ is a nitro group; R₄ is a hydrogen atom; R₂, R₆ and R₇ are a hydrogen atom; R₃ₐ and R_{3b} are a hydrogen atom, an alkyl group, an aryl group, or an acyl group); FIG. 3G (R₅ is a nitro group; R₄ is a phosphate group; R₄, R₆ and R₇ are a hydrogen atom; R₃ₐ and R_{3b} are a hydrogen atom, an alkyl group, an aryl group, or an acyl group); FIG. 3K (R₅ is a nitro group; R₄ is an ethyl group; R₄, R₆ and R₇ are a hydrogen atom; R₃ₐ and R_{3b} are a hydrogen atom, an alkyl group, an aryl group, or an acyl group); and FIG. 3O (R₅ is a nitro group; R₄ is an O-ethyl group; R₄, R₆ and R₇ are a hydrogen atom; R₃ₐ and R_{3b} are a hydrogen atom, an alkyl group, an aryl group, or an acyl group)).

Continuing to refer to the chemical compound having formula (I), in one embodiment, Re can be a nitro group, and each of R₂, R₅ and R₇ can be a hydrogen atom or an alkyl, O-alkyl or O-aryl group, and R₄ can be a phosphate group, a hydrogen atom, a hydroxy group, or an alkyl, O-alkyl or O-aryl group (see: the example nitrated psilocybin derivatives shown in FIG. 3D (R₆ is a nitro group; R₄ is a phosphate group; R₂, R₅ and R₇ are a hydrogen atom; R₃ₐ and R_{3b} are a hydrogen atom, an alkyl group, an aryl group, or an acyl group) and FIG. 3H (Re is a nitro group; R₄ is a phosphate group; R₂, R₅ and R₇ are a hydrogen atom; R₃ₐ and R_{3b} are a hydrogen atom, an alkyl group, an aryl group, or an acyl group); FIG. 3L (R₆ is a nitro group; R₄ is a methyl group; R₂, R₅ and R₇ are a hydrogen atom; R₃ₐ and R_{3b} are a hydrogen atom, an alkyl group, an aryl group, or an acyl group); and FIG. 3P (R₆ is a nitro group; R₄ is an O-methyl group; R₂, R₅ and R₇ are a hydrogen atom; R₃ₐ and R_{3b} are a hydrogen atom, an alkyl group, an aryl group, or an acyl group).

Continuing to refer to the chemical compound having formula (I), in one embodiment, R₇ can be a nitro group, and each of R₂, R₅ and R₆ can be a hydrogen atom or an alkyl, O-alkyl or O-aryl group, and R₄ can be a phosphate group, a hydrogen atom, a hydroxy group, or an alkyl, O-alkyl or O-aryl group (see: the example nitrated psilocybin derivatives shown in FIG. 3E (R₇ is a nitro group; R₄ is a hydrogen atom; R₂, R₅ and R₆ are a hydrogen atom R₃ₐ and R_{3b} are a hydrogen atom, an alkyl group, an aryl group, or an acyl group); FIG. 3I (R₇ is a nitro group; R₄ is a phosphate group; R₂, R₅ and R₆ are a hydrogen atom; R₃ₐ and R_{3b} are a hydrogen atom, an alkyl group, an aryl group, or an acyl group); FIG. 3M (R₇ is a nitro group; R₄ is a propyl group; R₂, R₅ and R₆ are a hydrogen atom; R₃ₐ and R_{3b} are a hydrogen atom, an alkyl group, an aryl group, or an acyl group); and FIG. 3O (R₇ is a nitro group; R₄ is an O-propyl group; R₂, R₅ and R₆ are a hydrogen atom; R₃ₐ and R_{3b} are a hydrogen atom, an alkyl group, an aryl group, or an acyl group).

In some embodiments, two of R₂, R₄, R₅, R₆ and R₇ of the chemical compound having formula (I) can be nitro groups. Thus, continuing to refer to the chemical compound having formula (I), in one embodiment, two of R₂, R₄, R₅, Re and R₇ can be a nitro group, wherein each non-nitrated R₂, R₅, R₆ and R₇ is a hydrogen atom or an alkyl, O-alkyl or O-aryl group, and wherein R₄, when it is not a nitro group, is a phosphate group, a hydrogen atom, a hydroxy group, or an alkyl, O-alkyl or O-aryl group.

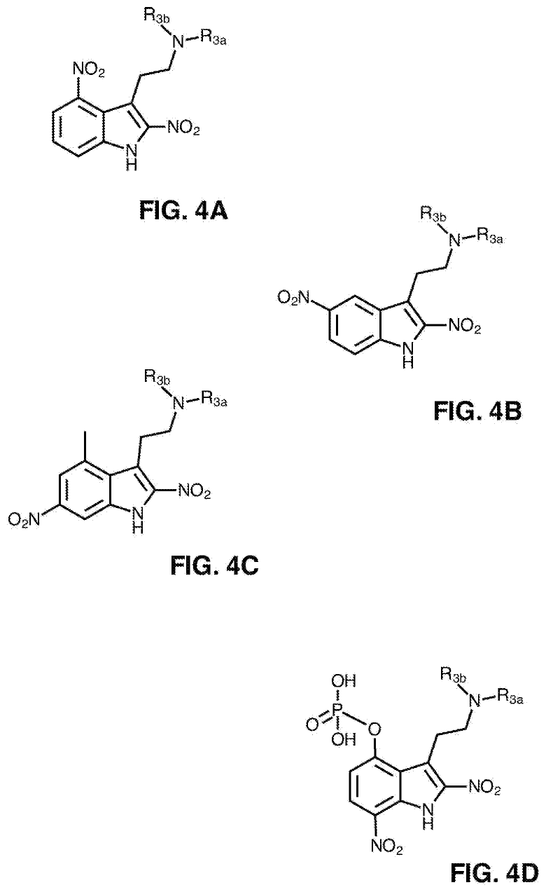

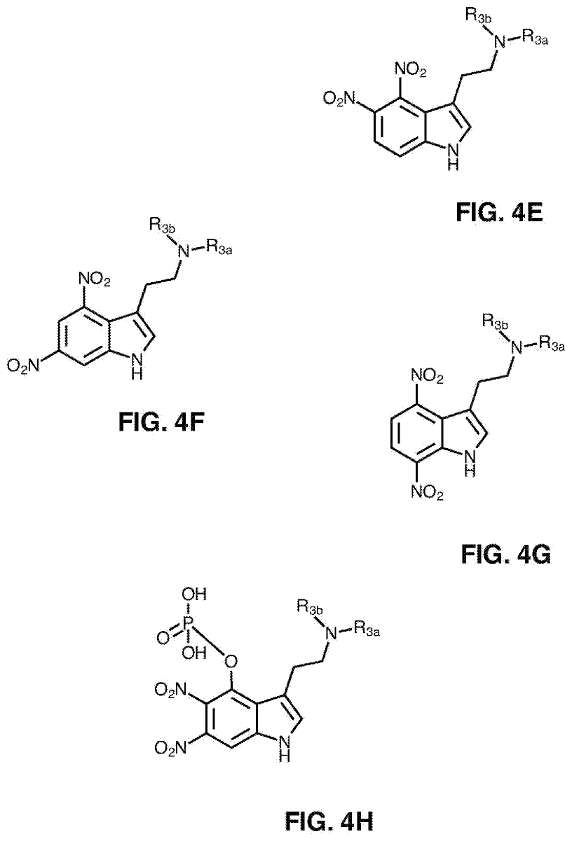

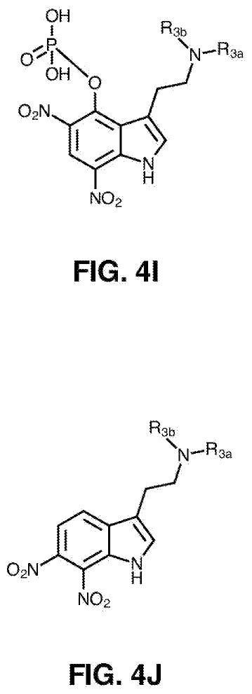

Still continuing to refer to the chemical compound having formula (I), in one embodiment, R₂ and R₄ can be nitro groups and R₅, R₆ and R₇ can be a hydrogen atom or an alkyl, O-alkyl or O-aryl group (see: the example nitrated psilocybin derivative shown in FIG. 4A (R₂ and R₄ are each a nitro group; R₅, Re and R₇ are hydrogen atoms; and R₃ₐ and R_{3b} are a hydrogen atom, an alkyl group, an aryl group, or an acyl group)).

Continuing to refer to the chemical compound having formula (I), in one embodiment, R₂ and R₅ can be nitro groups, and R₆ and R₇ can be a hydrogen atom or an alkyl, O-alkyl or O-aryl group, and R₄ can be a phosphate group, a hydrogen atom, a hydroxy group, or an alkyl, O-alkyl or O-aryl group (see: the example nitrated psilocybin derivative shown in FIG. 4B (R₂ and R₅ are each a nitro group; R₄, R₆ and R₇ are hydrogen atoms; and R₃ₐ and R_{3b} are a hydrogen atom, an alkyl group, an aryl group, or an acyl group)).

Continuing to refer to the chemical compound having formula (I), in one embodiment, R₂ and R₆ can nitro groups, and R₅ and R₇ can be a hydrogen atom or an alkyl, O-alkyl or O-aryl group, and R₄ can be a phosphate group, a hydrogen atom, a hydroxy group, or an alkyl, O-alkyl or O-aryl group (see: the example nitrated psilocybin derivative shown in FIG. 4C (R₂ and R₆ are each a nitro group; R₄ is a methyl group, R₅ and R₇ are hydrogen atoms; and R₃ₐ and R_{3b} are a hydrogen atom, an alkyl group, an aryl group, or an acyl group)).

Continuing to refer to the chemical compound having formula (I), in one embodiment, R₂ and R₇ can be nitro groups, and R₅ and R₆ can be a hydrogen atom or alkyl, O-alkyl or O-aryl group, and R₄ can be a phosphate group, a hydrogen atom, a hydroxy group, or an alkyl, O-alkyl or O-aryl group (see: the example nitrated psilocybin derivative shown in FIG. 4D (R₂ and R₇ are each nitro groups; R₄ is a phosphate group; R₅ and R₆ are hydrogen atoms; and R₃ₐ and R_{3b} are a hydrogen atom, an alkyl group, an aryl group, or an acyl group)).

In one embodiment, R₄ and R₅ can be nitro groups, R₂, R₆ and R₇ can be a hydrogen atom or alkyl, O-alkyl or O-aryl group (see: the example nitrated psilocybin derivative shown in FIG. 4E (R₄ and R₅ are each nitro groups; R₂, Re and R₇ are hydrogen atoms; and R₃ₐ and R_{3b} are a hydrogen atom, an alkyl group, an aryl group, or an acyl group)).

Continuing to refer to the chemical compound having formula (I), in one embodiment, R₄ and R₆ can be nitro groups, and R₂, R₅ and R₇ can be a hydrogen atom or an alkyl, O-alkyl or O-aryl group (see: the example nitrated psilocybin derivative shown in FIG. 4F (R₄ and R₆ are each nitro groups; R₂, R₅ and R₇ are hydrogen atoms; and R₃ₐ and R_{3b} are a hydrogen atom, an alkyl group, an aryl group, or an acyl group)).

Continuing to refer to the chemical compound having formula (I), in one embodiment, R₄ and R₇ can be nitro groups, and R₂, R₅ and R₆ can be a hydrogen atom or alkyl, O-alkyl or O-aryl group (see: the example hydroxy psilocybin derivative shown in FIG. 4G (R₄ and R₇ are each nitro groups; R₂, R₅ and R₆ are hydrogen atoms; and R₃ₐ and R_{3b} are a hydrogen atom, an alkyl group, an aryl group, or an acyl group)).

Continuing to refer to the chemical compound having formula (I), in one embodiment, R₅ and R₆ can be nitro groups and R₂ and R₇ can be a hydrogen atom or alkyl, O-alkyl or O-aryl group and R₄ can be a phosphate group, a hydrogen atom, a hydroxy group, or an alkyl, O-alkyl or O-aryl group (see: the example nitrated psilocybin derivative shown in FIG. 4H (R₅ and R₆ are each nitro groups; R₄ is a phosphate group; R₂ and R₇ are hydrogen atoms; and R₃ₐ and R_{3b} are a hydrogen atom, an alkyl group, an aryl group, or an acyl group)).

Continuing to refer to the chemical compound having formula (I), in one embodiment, R₅ and R₇ can be nitro groups, and R₂ and R₆ can be a hydrogen atom or alkyl, O-alkyl or O-aryl group and R₄ can be a phosphate group, a hydrogen atom, a hydroxy group, or an alkyl, O-alkyl or O-aryl group (see: the example nitrated psilocybin derivative shown in FIG. 4I (R₅ and R₇ are each nitro groups; R₄ is a phosphate group; R₂ and R₆ are hydrogen atoms; and R₃ₐ and R_{3b} are a hydrogen atom, an alkyl group, an aryl group, or an acyl group)).

Continuing to refer to the chemical compound having formula (I), in one embodiment, R₆ and R₇ can be nitro groups, and R₂ and R₅ can be a hydrogen atom or alkyl, O-alkyl or O-aryl group and R₄ can be a phosphate group, a hydrogen atom, a hydroxy group, or an alkyl, O-alkyl or O-aryl group (see: the example nitrated psilocybin derivative shown in FIG. 4J (R₆ and R₇ are each nitro groups; R₂, R₄ and R₅ are hydrogen atoms; and R₃ₐ and R_{3b} are a hydrogen atom, an alkyl group, an aryl group, or an acyl group)).

Referring again to the chemical compound having formula (I), in one further embodiment, three of R₂, R₄, R₅, R₆ and R₇ can be a nitro group, wherein the non-nitrated R₂, R₅, R₆, or R₇ substituents are a hydrogen atom or an alkyl, O-alkyl or O-aryl group, and wherein R₄, when it is not a nitro group, is a phosphate group, a hydrogen atom, a hydroxy group, or an alkyl, O-alkyl or O-aryl group.

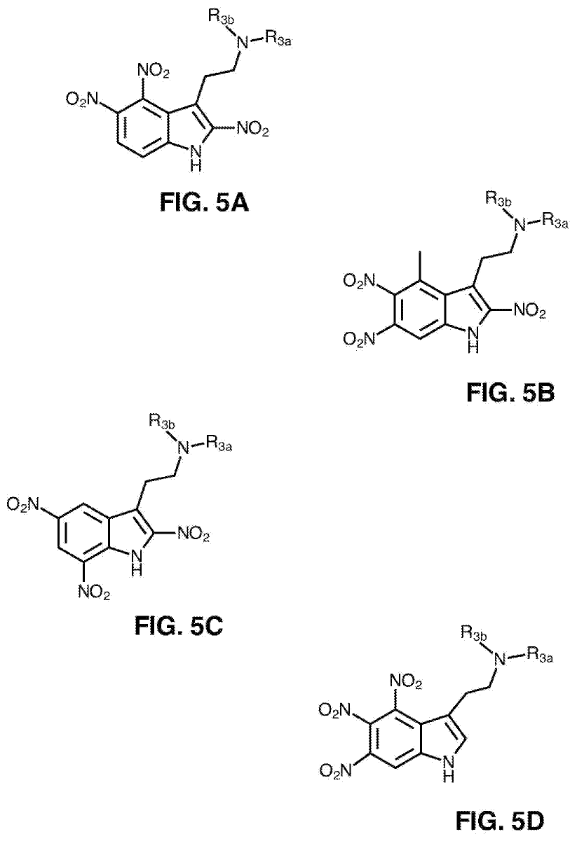

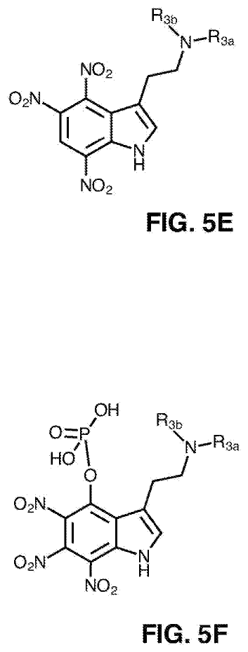

Thus, referring to the chemical compound having formula (I) again, in one embodiment R₂, R₄, and R₅ can be a nitro group, and R₆ and R₇ can be a hydrogen atom, an alkyl, O-alkyl or O-aryl group (see: the example nitrated psilocybin derivative shown in FIG. 5A (R₂, R₄ and R₅ are each nitro groups; Re and R₇ are hydrogen atoms; and R₃ₐ and R_{3b} are a hydrogen atom, an alkyl group, an aryl group, or an acyl group)).

Referring to the chemical compound having formula (I), in one embodiment, R₂, R₅, and R₆ can be a nitro groups, and R₇ can be a hydrogen atom, an alkyl, O-alkyl or O-aryl group, and R₄ can a phosphate group, a hydrogen atom, a hydroxy group, or an alkyl, O-alkyl or O-aryl group (see: the example nitrated psilocybin derivative shown in FIG. 5B (R₂, R₅ and R₆ are each nitro groups; R₄ is a methyl group; R₇ is a hydrogen atom; and R₃ₐ and R_{3b} are a hydrogen atom, an alkyl group, an aryl group, or an acyl group)).

Referring to the chemical compound having formula (I), in one embodiment, R₂, R₅, and R₇ can be a nitro group and R₆ can be a hydrogen atom or an alkyl, O-alkyl or O-aryl group, and R₄ can a phosphate group, a hydrogen atom, a hydroxy group, or an alkyl, O-alkyl or O-aryl group (see: the example nitrated psilocybin derivative shown in FIG. 5C (R₂, R₅ and R₇ are each nitro groups; R₄ and R₆ are hydrogen atoms; and R₃ₐ and R_{3b} are a hydrogen atom, an alkyl group, an aryl group, or an acyl group)).

Referring to the chemical compound having formula (I), in one embodiment, R₄, R₅, and R₆ can be a nitro group, and R₂ and R₇ can be a hydrogen atom or an alkyl, O-alkyl or O-aryl group (see: the example nitrated psilocybin derivative shown in FIG. 5D (R₄, R₅ and R₆ are each a nitro group; R₂ and R₇ are hydrogen atoms; and R₃ₐ and R_{3b} are a hydrogen atom, an alkyl group, an aryl group, or an acyl group)).

Referring to the chemical compound having formula (I), in one embodiment, R₄, R₅, and R₇ can be a nitro group, and R₂ and R₆ can be a hydrogen atom or an alkyl, O-alkyl or O-aryl group (see: the example nitrated psilocybin derivative shown in FIG. 5E (R₄ R₅ and R₇ are each a nitro group; R₂ and R₆ are hydrogen atoms; and R₃ₐ and R_{3b} are a hydrogen atom, an alkyl group, an aryl group, or an acyl group)).

Referring to the chemical compound having formula (I), in one embodiment, R₅, R₆, and R₇ can a nitro group, and R₂ can be a hydrogen atom or an alkyl, O-alkyl or O-aryl group, and R₄ can be a phosphate group, a hydrogen atom, a hydroxy group, or an alkyl, O-alkyl or O-aryl group (see: the example nitrated psilocybin derivative shown in FIG. 5F (R₅, R₆ and R₇ are each a nitro group; R₄ is a phosphate group; R₂ is a hydrogen atom; and R₃ₐ and R_{3b} are a hydrogen atom, an alkyl group, an aryl group, or an acyl group)).

Referring again to the chemical compound having formula (I), in one embodiment, four of R₂, R₄, R₅, R₆ and R₇ can be a nitro group and wherein the non-nitrated R₂, R₅, R₆, or R₇ is a hydrogen atom or an alkyl, O-alkyl or O-aryl group, and wherein R₄, when it is not a nitro group, is a phosphate group, a hydrogen atom, a hydroxy group, or an alkyl, O-alkyl or O-aryl group.

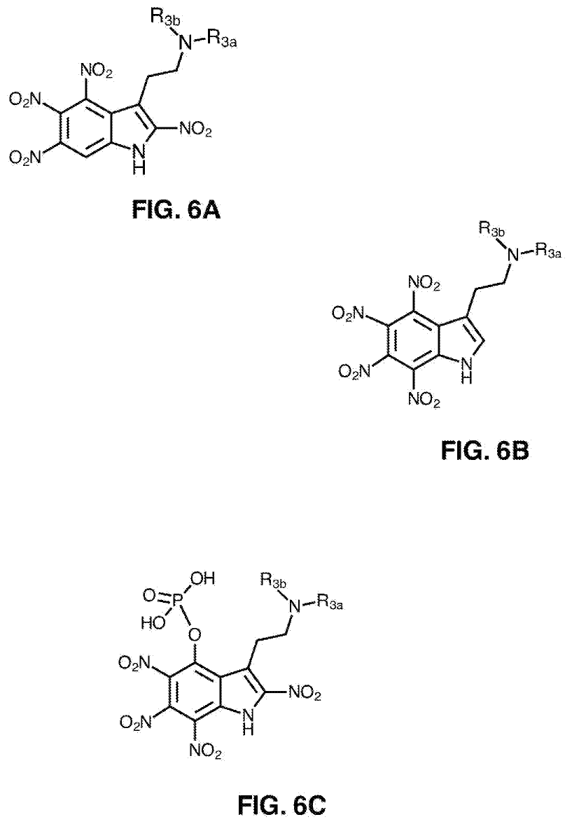

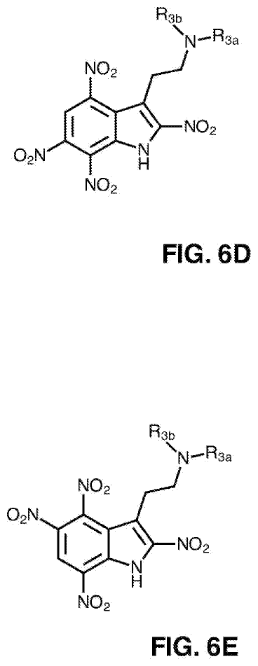

Thus, referring to the chemical compound having formula (I), in one embodiment, R₂, R₄, R₅ and R₆ can be a nitro group and R₇ can be a hydrogen atom or an alkyl, O-alkyl or O-aryl group (see: the example nitrated psilocybin derivative shown in FIG. 6A (R₂, R₄, R₅ and R₆ are each nitro groups; R₇ is a hydrogen atom; and R₃ₐ and R_{3b} are a hydrogen atom, an alkyl group, an aryl group, or an acyl group)).

Referring to the chemical compound having formula (I), in one embodiment, R₄, R₅, R₆ and R₇ can be a nitro group and R₂ can be a hydrogen atom or an alkyl, O-alkyl or O-aryl group (see: the example nitrated psilocybin derivative shown in FIG. 6B (R₄, R₅, R₆ and R₇ are each nitro groups; R₂ is a hydrogen atom; and R₃ₐ and R_{3b} are a hydrogen atom, an alkyl group, an aryl group, or an acyl group)).

Referring to the chemical compound having formula (I), in one embodiment, R₂, R₅, R₆ and R₇ can be a nitro group, R₄ can be a phosphate group, a hydrogen atom, a hydroxy group, or an alkyl, O-alkyl or O-aryl group (see: the example nitrated psilocybin derivative shown in FIG. 6C (R₂, R₅, R₆ and R₇ are each nitro groups; R₄ is a phosphate group; and R₃ₐ and R_{3b} are a hydrogen atom, an alkyl group, an aryl group, or an acyl group)).

Referring to the chemical compound having formula (I), in one embodiment R₂, R₄, R₆ and R₇ can be a nitro group and R₅ can be a hydrogen atom or an alkyl, O-alkyl or O-aryl group (see: the example nitrated psilocybin derivative shown in FIG. 6D (R₂, R₄, R₆, and R₇ are nitro groups; R₅ is a hydrogen atom; and R₃ₐ and R_{3b} are a hydrogen atom, an alkyl group, an aryl group, or an acyl group)).

Referring to the chemical compound having formula (I), in one embodiment R₂, R₄, R₅ and R₇ can be nitro groups and R₆ can be a hydrogen atom or an alkyl, O-alkyl or O-aryl group (see: the example nitrated psilocybin derivative shown in FIG. 6E (R₂, R₄, R₅ and R₇ are each nitro groups; R₆ is a hydrogen atom; and R₃ₐ and R_{3b} are a hydrogen atom, an alkyl group, an aryl group, or an acyl group)).

In one embodiment, all five of R₂, R₄, R₅, R₆ and R₇ can be a nitro group.

It is noted that, in a further aspect hereof, R_{3A} and R_{3B} can be a hydrogen atom, an alkyl group, an aryl group or an acyl group. Thus, for example, R_{3A} and R_{3B} can each be a hydrogen atom, or R_{3A} and R_{3B} can each be an alkyl group, such as a methyl group, ethyl group, propyl group, or longer chain alkyl group, or R_{3A} and R_{3B} can each be an aryl group, such as a phenyl group or a naphthyl group, or R_{3A} and R_{3B} can each be an acyl group, such as an acetyl group. Furthermore, one of R_{3A} and R_{3B} can be a hydrogen atom, and one of R_{3A} and R_{3B} can be an alkyl group, and aryl group, or an acyl group. Furthermore, RA and R_{3B} can be an aryl group and an alkyl group, an aryl group and an acyl group, and an acyl group.

Furthermore, in one embodiment, a nitrated psilocybin derivative according to the present disclosure can be a chemical compound having the formula (III):

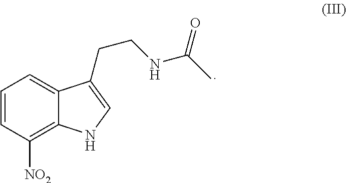 [SMILES: CC(=O)NCCc1c[nH]c2c([N+](=O)[O-])cccc12]

Furthermore, in one embodiment, a nitrated psilocybin derivative according to the present disclosure can be a chemical compound having the formula (IV):

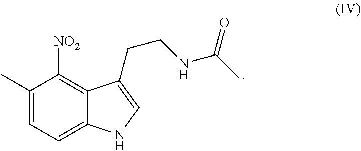 [SMILES: CC(=O)NCCc1c[nH]c2ccc(C)c([N+](=O)[O-])c12]

Furthermore, in one embodiment, a nitrated psilocybin derivative according to the present disclosure can be a chemical compound having the formula (V):

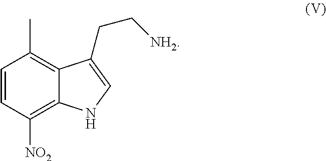 [SMILES: Cc1ccc([N+](=O)[O-])c2[nH]cc(CCN)c12]

Furthermore, in one embodiment, a nitrated psilocybin derivative according to the present disclosure can be a chemical compound having the formula (VI):

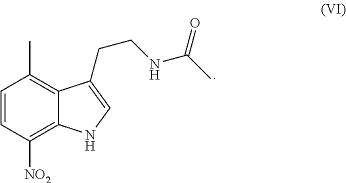 [SMILES: CC(=O)NCCc1c[nH]c2c([N+](=O)[O-])ccc(C)c12]

Furthermore, in one embodiment, a nitrated psilocybin derivative according to the present disclosure can be a chemical compound having the formula (VII):

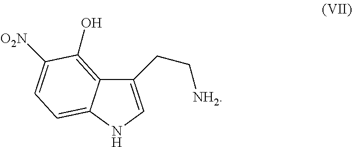 [SMILES: NCCc1c[nH]c2ccc([N+](=O)[O-])c(O)c12]

Furthermore, in one embodiment, a nitrated psilocybin derivative according to the present disclosure can be a chemical compound having the formula (VIII):

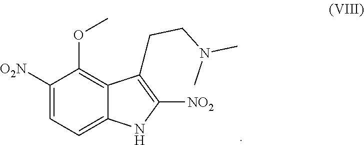 [SMILES: COc1c([N+](=O)[O-])ccc2[nH]c([N+](=O)[O-])c(CCN(C)C)c12]

Furthermore, in one embodiment, a nitrated psilocybin derivative according to the present disclosure can be a chemical compound having the formula (IX):

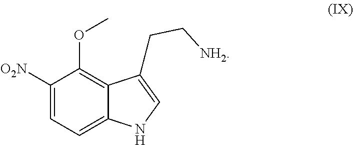 [SMILES: COc1c([N+](=O)[O-])ccc2[nH]cc(CCN)c12]

Furthermore, in one embodiment, a nitrated psilocybin derivative according to the present disclosure can be a chemical compound having the formula (X):

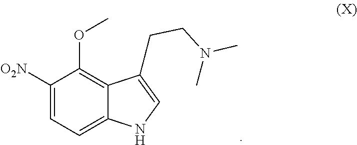 [SMILES: COc1c([N+](=O)[O-])ccc2[nH]cc(CCN(C)C)c12]

Furthermore, in one embodiment, a nitrated psilocybin derivative according to the present disclosure can be a chemical compound having the formula (XXVIII):

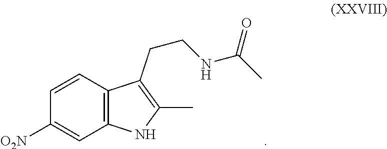 [SMILES: CC(=O)NCCc1c(C)[nH]c2cc([N+](=O)[O-])ccc12]

Furthermore, in one embodiment, a nitrated psilocybin derivative according to the present disclosure can be a chemical compound having the formula (XXIX):

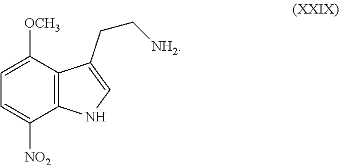 [SMILES: COc1ccc([N+](=O)[O-])c2[nH]cc(CCN)c12]

Furthermore, it is noted that the nitrated psilocybin derivatives of the present disclosure include salts thereof, including pharmaceutically acceptable salts. Thus, the nitrogen atom of the 2-aminoethyl group extending in turn from the C₃ atom may be protonated, and the positive charge may be balanced by, for example, chloride or sulfate ions, to thereby form a chloride salt or a sulfate salt. Furthermore, in compounds wherein R₄ is a phosphate group, the phosphate group may be de-protonated, and the negative charge may be balanced by, for example, sodium ions or potassium ions, to thereby form a sodium salt or a potassium salt.

Furthermore, it is noted that when R₄ is a phosphate group, the term nitrated psilocybin derivative also includes compounds having the formula (XI):

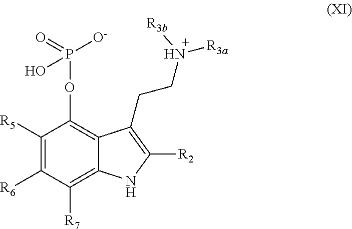

wherein at least one of R₂, R₅, R₆, or R₇ is a nitro group, and wherein any R₂, R₅, R₆, or R₇ which are not a nitro group are a hydrogen atom or an alkyl, O-alkyl or O-aryl group, and wherein R_{3A} and R_{3B} are a hydrogen atom, an alkyl group, and aryl group or an acyl group. Further included are salts of nitrated psilocybins having the formula (VII), such as a sodium salt, a potassium salt etc.

Thus, to briefly recap, the present disclosure provides nitrated psilocybin derivatives. The disclosure provides, in particular, a chemical compound or salt thereof having formula (I):

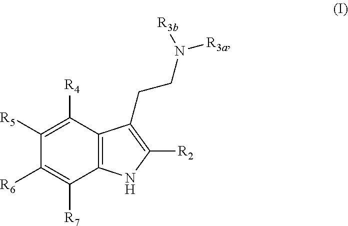

wherein in an aspect, at least one of R₂, R₄, R₅, R₆, or R₇ is a nitro group. In an aspect, in formula (I), each non-nitrated R₂, R₅, R₆, or R₇ is a hydrogen atom or an alkyl, O-alkyl or O-aryl group. In a further aspect, in formula (I), R₄ when it is not nitrated is a hydrogen atom, an alkyl, O-alkyl or O-aryl group, a hydroxy group, or a phosphate group. Yet in a further aspect, R_{3A} and R_{3B} are a hydrogen atom, an alkyl group, an aryl group, or an acyl group.

In one embodiment of the disclosure, a chemical compound or salt thereof having formula (I) is included:

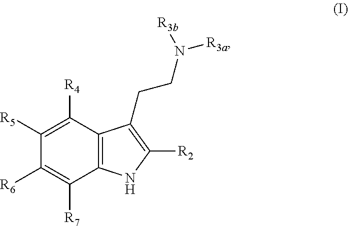

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, alkyl, O-alkyl or O-aryl group, a hydroxy group, or a phosphate group, and wherein R_{3A} and R_{3B} are a hydrogen atom, an alkyl group, an aryl group, or an acyl group.

In one embodiment, at least one of R₂, R₄, R₅, R₆, or R₇ is a nitro group, and wherein each non-nitrated R₂, R₅, R₆, or R₇ is a hydrogen atom or a (C₁-C₂₀)-alkyl group or (C₁-C₂₀)—O-alkyl group. In another embodiment, each non-nitrated R₂, R₅, R₆, or R₇ is a hydrogen atom, a methyl group, ethyl group, a propyl group, an O-methyl group, an O-ethyl group, an O-propyl group or a benzyloxy group.

In another embodiment, each non-nitrated R₂, R₅, R₆, or R₇ is a hydrogen atom or a (C₁-C₁₀)-alkyl group or (C₁-C₁₀)—O-alkyl group. In another embodiment, each non-nitrated R₂, R₅, R₆, or R₇ is a hydrogen atom, a methyl group, ethyl group, a propyl group, an O-methyl group, an O-ethyl group, an O-propyl group, or a benzyloxy group.

In another embodiment, each non-nitrated R₂, R₅, R₆, or R₇ is a hydrogen atom or a (C₁-C₆)-alkyl group or (C₁-C₆)—O-alkyl group. In another embodiment, each non-nitrated R₂, R₅, R₆, or R₇ is a hydrogen atom, a methyl group, ethyl group, a propyl group, an O-methyl group, an O-ethyl group, an O-propyl group, or a benzyloxy group.

In another embodiment, each non-nitrated R₂, R₅, R₆, or R₇ is a hydrogen atom or a (C₁-C₂₀)—O-aryl group or (C₁-C₁₀)—O-aryl group. In another embodiment, In another embodiment, each non-nitrated R₂, R₅, R₆, or R₇ is a hydrogen atom, a methyl group, ethyl group, a propyl group, an O-methyl group, an O-ethyl group, an O-propyl group, or a benzyloxy group.

In another embodiment, when R₄ is not nitrated, R₄ is a hydrogen atom, a (C₁-C₂₀)-alkyl group, (C₁-C₂₀)—O-alkyl group or (C₁-C₂₀)—O-aryl group, a hydroxy group, or a phosphate group. In another embodiment, when R₄ is not nitrated, R₄ is a hydrogen atom, a (C₁-C₁₀)-alkyl group, (C₁-C₁₀)—O-alkyl group or (C₁-C₁₀)—O-aryl group, a hydroxy group, or a phosphate group. In another embodiment, when R₄ is not nitrated, R₄ is a hydrogen atom, a (C₁-C₆)-alkyl group or (C₁-C₆)—O-alkyl group, a hydroxy group, or a phosphate group. In another embodiment, when R₄ is not nitrated, R₄ is a hydrogen atom, a methyl group, an ethyl group, a propyl group, a phosphate group, an O-methyl group, an O-ethyl group, an O-propyl group or a benzyloxy group.

In another embodiment, R_{3A} and R_{3B} are a hydrogen atom, a (C₁-C₂₀)-alkyl group, a (C₆-C₁₄)-aryl group, or a —C(═O)(C₁-C₂₀)-alkyl group. In another embodiment, R_{3A} and R_{3B} are a hydrogen atom, a (C₁-C₁₀)-alkyl group, a (C₆-C₁₀)-aryl group, or a —C(═O)(C₁-C₁₀)-alkyl group. In another embodiment, R_{3A} and R_{3B} are a hydrogen atom, a (C₁-C₆)-alkyl group, a phenyl group, or a —C(═O)(C₁-C₆)-alkyl group. In another embodiment, R_{3A} and R_{3B} are a hydrogen atom, a methyl group, an ethyl group, a propyl group, a phenyl group, —C(═O)—CH₃, —C(═O)—CH₂CH₃, or —C(═O)—CH₂CH₂CH₃.

In one embodiment of the disclosure, a chemical compound or salt thereof having formula (I) is included:

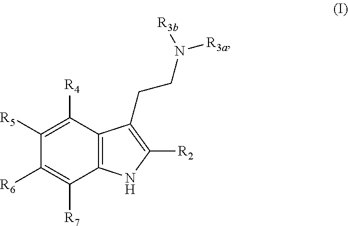

wherein
R₂, R₅, R₆, and R₇ are independently or simultaneously H, an alkyl, O-alkyl or O-aryl group or a nitro group, R_{3A} and R_{3B} are a hydrogen atom, an alkyl group, an aryl group, or an acyl group; and R₄ is hydrogen atom, an alkyl, O-alkyl or O-aryl group, a nitro group, a hydroxy group, or a phosphate group; wherein at least one of R₂, R₄ R₅, R₆, and R₇ is a nitro group.

In one embodiment, R₂, R₅, R₆, and R₇ are independently or simultaneously H, (C₁-C₂₀)-alkyl group or (C₁-C₂₀)—O-alkyl group or (C₁-C₂₀)—O-aryl group or a nitro group. In one embodiment, R₂, R₅, R₆, and R₇ are independently or simultaneously H, (C₁-C₁₀)-alkyl group or (C₁-C₁₀)—O-alkyl group or (C₁-C₁₀)—O-aryl group or a nitro group. In one embodiment, R₂, R₅, R₆, and R₇ are independently or simultaneously H, (C₁-C₆)-alkyl group or (C₁-C₆)—O-alkyl group or a nitro group. In one embodiment, R₂, R₅, R₆, and R₇ are independently or simultaneously H, methyl, ethyl, propyl, O-methyl, O-ethyl, O-propyl, benzyloxy, or a nitro group.

In one embodiment, R₄ is H, (C₁-C₂₀)-alkyl group or (C₁-C₂₀)—O-alkyl group or (C₁-C₂₀)—O-aryl group, a nitro group or a phosphate group. In one embodiment, R₄ is H, (C₁-C₁₀)-alkyl group or (C₁-C₁₀)—O-alkyl group (C₁-C₁₀)—O-aryl group or a nitro group or a phosphate group. In one embodiment, R₄ is H, (C₁-C₆)-alkyl group or (C₁-C₆)—O-alkyl group, a nitro group, a hydroxy group, or a phosphate group. In one embodiment, R₄ is H, methyl, ethyl, propyl, O-methyl, O-ethyl, O-propyl, benzyloxy, a nitro group, a hydroxy group, or a phosphate group.

In another embodiment, R_{3A} and R_{3B} are a hydrogen atom, a (C₁-C₂₀)-alkyl group, a (C₆-C₁₄)-aryl group, or a —C(═O)(C₁-C₂₀)-alkyl group. In another embodiment, R_{3A} and R_{3B} are a hydrogen atom, a (C₁-C₁₀)-alkyl group, a (C₆-C₁₀)-aryl group, or a —C(═O)(C₁-C₁₀)-alkyl group or O-alkyl group. In another embodiment, R_{3A} and R_{3B} are a hydrogen atom, a (C₁-C₆)-alkyl group, a phenyl group, or a —C(═O)(C₁-C₆)-alkyl group. In another embodiment, R_{3A} and R_{3B} are a hydrogen atom, a methyl group, an ethyl group, a propyl group, a phenyl group, —C(═O)—CH₃, —C(═O)—CH₂CH₃, or —C(═O)—CH₂CH₂CH₃.

The nitrated psilocybin derivatives of the present disclosure may be used to prepare a pharmaceutical or recreational drug formulation. Thus in one embodiment, the present disclosure further provides in another aspect, pharmaceutical and recreational drug formulations comprising nitrated psilocybin derivatives. Accordingly, in one aspect, the present disclosure provides in a further embodiment a pharmaceutical or recreational drug formulation comprising a chemical compound having formula (I):

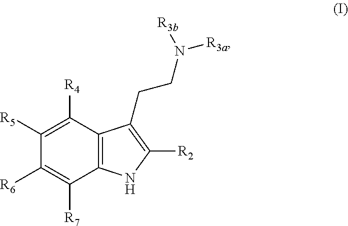

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen atom an alkyl group, an aryl group, or an acyl group, or a slat of the chemical compound, together with a diluent, carrier or excipient.

The dose when using the compounds of the present disclosure can vary within wide limits, and as is customary and is known to those of skill in the art, the dose can be tailored to the individual conditions in each individual case. The dose depends, for example, on the nature and severity of the illness to be treated, on the condition of the patient, on the compound employed or on whether an acute or chronic disease state is treated or prophylaxis is conducted, on the mode of delivery of the compound, or on whether further active compounds are administered in addition to the compounds of the present disclosure. Representative doses of the present disclosure include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to about 100 mg, about 0.001 mg to about 50 mg, and about 0.001 mg to about 25 mg. Representative doses of the present disclosure include, but are not limited to, about 0.0001 to about 1,000 mg, about 10 to about 160 mg, about 10 mg, about 20 mg, about 40 mg, about 80 mg or about 160 mg. Multiple doses may be administered during the day, especially when relatively large amounts are deemed to be needed, for example 2, 3 or 4, doses. Depending on the subject and as deemed appropriate from the patient's physician or care giver it may be necessary to deviate upward or downward from the doses described herein.

The pharmaceutical or recreational drug formulations may be prepared as liquids, tablets, capsules, microcapsules, nanocapsules, trans-dermal patches, gels, foams, oils, aerosols, nanoparticulates, powders, creams, emulsions, micellar systems, films, sprays, ovules, infusions, teas, decoctions, suppositories, etc. and include a pharmaceutically acceptable salt or solvate of the nitrated psilocybin compound together with an excipient. The term “excipient” as used herein means any ingredient other than the chemical compound of the disclosure. As will readily be appreciated by those of skill in art, the selection of excipient may depend on factors such as the particular mode of administration, the effect of the excipient on solubility of the chemical compounds of the present disclosure and methods for their preparation will be readily apparent to those skilled in the art. Such compositions and methods for their preparation may be found, for example, in “Remington's Pharmaceutical Sciences”, 22nd Edition (Pharmaceutical Press and Philadelphia College of Pharmacy at the University of the Sciences, 2012).

The pharmaceutical and drug formulations comprising the nitrated psilocybin derivatives of the present disclosure may be administered orally. Oral administration may involve swallowing, so that the compound enters the gastrointestinal tract, or buccal or sublingual administration may be employed by which the compound enters the blood stream directly from the mouth. Formulations suitable for oral administration include both solid and liquid formulations.

Solid formulations include tablets, capsules (containing particulates, liquids, microcapsules, or powders), lozenges (including liquid-filled lozenges), chews, multi- and nano-particulates, gels, solid solutions, liposomal preparations, microencapsulated preparations, creams, films, ovules, suppositories and sprays.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose.

Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80. When present, surface active agents may comprise from 0.2% (w/w) to 5% (w/w) of the tablet.

Tablets may further contain lubricants such as magnesium stearate, calcium stearate, zinc stearate, sodium stearyl fumarate, and mixtures of magnesium stearate with sodium lauryl sulphate. Lubricants generally comprise from 0.25% (w/w) to 10% (w/w), from 0.5% (w/w) to 3% (w/w) of the tablet.

In addition to the nitrated psilocybin derivative, tablets may contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1% (w/w) to 25% (w/w) or from 5% (w/w) to 20% (w/w) of the dosage form.

Other possible auxiliary ingredients include anti-oxidants, colourants, flavouring agents, preservatives and taste-masking agents.

For tablet dosage forms, depending on the desired effective amount of the chemical compound, the chemical compound of the present disclosure may make up from 1% (w/w) to 80% (w/w) of the dosage form, more typically from 5% (w/w) to 60% (w/w) of the dosage form.

Exemplary tablets contain up to about 80% (w/w) of the chemical compound, from about 10% (w/w) to about 90% (w/w) binder, from about 0% (w/w) to about 85% (w/w) diluent, from about 2% (w/w) to about 10% (w/w) disintegrant, and from about 0.25% (w/w) to about 10% (w/w) lubricant.

The formulation of tablets is discussed in “Pharmaceutical Dosage Forms: Tablets”, Vol. 1-Vol. 3, by CRC Press (2008).

The pharmaceutical and recreational drug formulations comprising the nitrated psilocybin derivatives of the present disclosure may also be administered directly into the blood stream, into muscle, or into an internal organ. Thus, the pharmaceutical and recreational drug formulations can be administered parenterally (for example, by subcutaneous, intravenous, intraarterial, intrathecal, intraventricular, intracranial, intramuscular, or intraperitoneal injection). Parenteral formulations are typically aqueous solutions which may contain excipients such as salts, carbohydrates and buffering agents (in one embodiment, to a pH of from 3 to 9), but, for some applications, they may be more suitably formulated as a sterile non-aqueous solution or as a dried form to be used in conjunction with a suitable vehicle such as sterile water.

Formulations comprising the nitrated psilocybin derivatives of the present disclosure for parenteral administration may be formulated to be immediate and/or modified release. Modified release formulations include delayed-, sustained-, pulsed-, controlled-, targeted and programmed release. Thus the chemical compounds of the disclosure may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The pharmaceutical or recreational drug formulations of the present disclosure also may be administered topically to the skin or mucosa, i.e. dermally or transdermally. Example pharmaceutical and recreational drug formulations for this purpose include gels, hydrogels, lotions, solutions, creams, ointments, dusting powders, cosmetics, oils, eye drops, dressings, foams, films, skin patches, wafers, implants, sponges, fibres, bandages and microemulsions. Liposomes may also be used. Example carriers include alcohol, water, mineral oil, liquid petrolatum, white petrolatum, glycerin, polyethylene glycol and propylene glycol. Penetration enhancers may be incorporate (see: for example, Finnin, B. and Morgan, T. M., 1999 J. Pharm. Sci, 88 (10), 955-958).

Other means of topical administration include delivery by electroporation, iontophoresis, phonophoresis, sonophoresis and microneedle or needle-free (e.g., Powderject™, Bioject™, etc.) injection.

Pharmaceutical and recreational drug formulations for inhalation or insufflation include solutions and suspensions in pharmaceutically acceptable, aqueous or organic solvents, or mixtures thereof, and powders. The liquid or solid pharmaceutical compositions can contain suitable pharmaceutically acceptable excipients. In some embodiments, the pharmaceutical compositions are administered by the oral or nasal respiratory route for local or systemic effect. Pharmaceutical compositions in pharmaceutically acceptable solvents can be nebulized by use of inert gases. Nebulized solutions can be inhaled directly from the nebulizing device or the nebulizing device can be attached to a face mask tent, or intermittent positive pressure breathing machine. Solution, suspension, or powder pharmaceutical compositions can be administered, e.g., orally or nasally, from devices that deliver the formulation in an appropriate manner.

In further embodiments, in which the nitrated psilocybin compounds of present disclosure are used as a recreational drug, the compounds may be included in compositions such as a food or food product, a beverage, a food seasoning, a personal care product, such as a cosmetic, perfume or bath oil, or oils (both for topical administration as massage oil, or to be burned or aerosolized). The chemical compounds of the present disclosure may also be included in a “vape” product, which may also include other drugs, such as nicotine, and flavorings.

The pharmaceutical formulations comprising the chemical compounds of the present disclosure may be used to treat a subject, and in particular to treat a psychiatric disorder in a subject. Accordingly, the present disclosure includes in a further embodiment, a method for treating a psychiatric disorder, the method comprising administering to a subject in need thereof a pharmaceutical formulation comprising a chemical compound or salt thereof having formula (I):

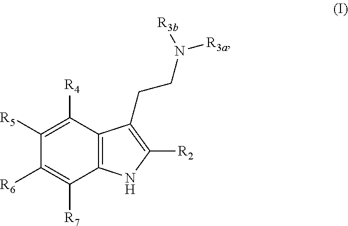

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen atom, an alkyl group, an aryl group, or an acyl group, together with a diluent, carrier or excipient.

Psychiatric disorders that may be treated include, for example, neurodevelopmental disorders such as intellectual disability, global development delay, communication disorders, autism spectrum disorder, and attention-deficit hyperactivity disorder (ADHD); bipolar and related disorders, such as mania, and depressive episodes; anxiety disorder, such as generalized anxiety disorder (GAD), agoraphobia, social anxiety disorder, specific phobias (natural events, medical, animal, situational, for example), panic disorder, and separation anxiety disorder; stress disorders, such as acute stress disorder, adjustment disorders, post-traumatic stress disorder (PTSD), and reactive attachment disorder; dissociative disorders, such as dissociative amnesia, dissociative identity disorder, and depersonalization/derealization disorder; somatoform disorders, such as somatic symptom disorders, illness anxiety disorder, conversion disorder, and factitious disorder; eating disorders, such as anorexia nervosa, bulimia nervosa, rumination disorder, pica, and binge-eating disorder; sleep disorders, such as narcolepsy, insomnia disorder, hypersomnolence, breathing-related sleep disorders, parasomnias, and restless legs syndrome; disruptive disorders, such as kleptomania, pyromania, intermittent explosive disorder, conduct disorder, and oppositional defiant disorder; depressive disorders, such as disruptive mood dysregulation disorder, major depressive disorder, persistent depressive disorder (dysthymia), premenstrual dysphoric disorder, substance/medication-induced depressive disorder, postpartum depression, and depressive disorder caused by another medical condition for example, psychiatric and existential distress within life-threatening cancer situations (ACS Pharmacol. Transl. Sci. 4: 553-562; J PsychiatrRes 137: 273-282); substance-related disorders, such as alcohol-related disorders, cannabis related disorders, inhalant-use related disorders, stimulant use disorders, and tobacco use disorders; neurocognitive disorders, such as delirium; schizophrenia; compulsive disorders, such as obsessive compulsive disorders (OCD), body dysmorphic disorder, hoarding disorder, trichotillomania disorder, excoriation disorder, substance/medication induced obsessive-compulsive disorder, and obsessive-compulsive disorder related to another medical condition; and personality disorders, such as antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder.

In an aspect, the compounds of the present disclosure may be used to be contacted with a 5-HT_{2A} receptor to thereby modulate the 5-HT_{2A} receptor. Such contacting includes bringing a compound of the present disclosure and 5-HT_{2A} receptor together under in vitro conditions, for example, by introducing the compounds in a sample containing a 5-HT_{2A} receptor, for example, a sample containing purified 5-HT_{2A} receptors, or a sample containing cells comprising 5-HT_{2A} receptors. In vitro conditions further include the conditions described in Example 3 hereof. Contacting further includes bringing a compound of the present disclosure and 5-HT_{2A} receptor together under in vivo conditions. Such in vivo conditions include the administration to an animal or human subject, for example, of a pharmaceutically effective amount of the compound of the present disclosure, when the compound is formulated together with a pharmaceutically active carrier, diluent or excipient, as hereinbefore described, to thereby treat the subject. Upon having contacted the 5-HT_{2A} receptor, the compound may activate the 5-HT_{2A} receptor or inhibit the 5-HT_{2A} receptor.

Thus, in a further aspect, the condition that may be treated in accordance herewith can be any 5-HT_{2A} receptor mediated disorder. Such disorders include, but are not limited to schizophrenia, psychotic disorder, attention deficit hyperactivity disorder, autism, and bipolar disorder.

The chemical compounds of the present disclosure may also be used as a feedstock material for other psilocybin derivatives. Thus in one embodiment, the chemical compounds of the present disclosure may be in used manufacture of a pharmaceutical or recreational drug formulation, wherein the manufacture may comprise derivatizing a chemical compound having the formula (I):

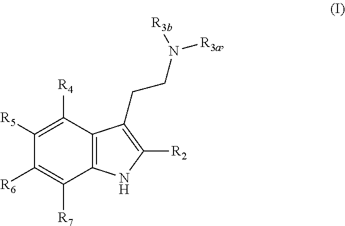

wherein, at least one of R₂, R₄, R₅, R₆ or R₇ is a nitro group, wherein each non-nitrated R₂, R₅, R₆, or R₇ is a hydrogen atom or alkyl, O-alkyl or O-aryl group, wherein R₄ when it is not nitrated is a phosphate group, a hydrogen atom, a hydroxy group, an alkyl, O-alkyl or O-aryl group, and wherein R_{3A} and R_{3B} are a hydrogen atom, an alkyl group, an aryl group, or an acyl group, or a salt of the chemical compound.

In order to use the compound having formula (I) as a feedstock, one or more nitro groups may be substituted by any atoms or groups, for example hydrocarbon groups. Those of skill in the art will be generally familiar with methods that may be used to substitute nitro groups. In this respect, guidance may be found in Schnepel C. et al. (2017) Chem. Eur. J. 23:12064-12086; Durak L. J. et al. (2016) ACS Catal. 6: 1451; Runguphan W. et al. (2013) Org Lett 15: 2850; Corr M. J. et al. (2017) Chem. Sci. 8: 2039; and Roy A. D. et al. Chem. Comm. 4831.

Turning now to methods of making the nitrated psilocybin derivatives, it is noted that the psilocybin compounds of the present disclosure may be prepared in any suitable manner, including any organic chemical synthesis methods, biosynthetic methods, or a combination thereof.

One suitable method of making the nitrated psilocybin derivatives of the present disclosure comprises a method of making a nitrated psilocybin derivative comprising:

reacting a reactant psilocybin derivative compound or a salt thereof having the formula (II):

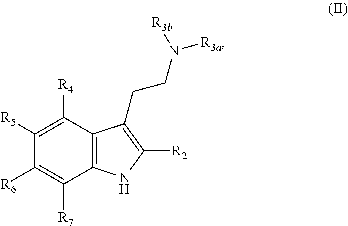

wherein, at least one of R₂, R₄, R₅, R₆, or R₇ is a reactant group, and wherein each R₂, R₅, R₆, or R₇ which is not a reactant group is a hydrogen atom or an alkyl, O-alkyl or O-aryl group, wherein R₄ when it is not a reactant group is a phosphate group, a hydrogen atom, a hydroxy group, an alkyl, O-alkyl or O-aryl group, and wherein R_{3A} and R_{3B} are a hydrogen atom, an alkyl group, an aryl group or an acyl group, with a nitro group donating compound under conditions sufficient to form a chemical compound having formula (I):

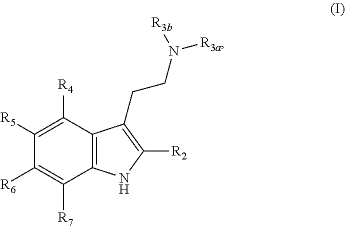

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, alkyl, O-alkyl or O-aryl group, a hydroxy group, or a phosphate group, and wherein R_{3A} and R_{3B} are a hydrogen atom, an alkyl group, an aryl group, or an acyl group.

Thus, in an aspect hereof, a reactant psilocybin derivative and a nitro group donating compound are provided, and the reactant psilocybin derivative and the nitro group donating compound are contacted to react in a chemical reaction resulting in the formation of a nitrated psilocybin derivative compound.

Suitable reactant psilocybin derivative compounds include compounds comprising an indole prototype structure (see: FIG. 2), including, for example, a chemical compound having formula (II)

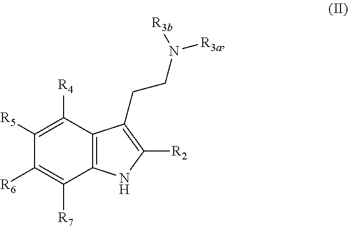

wherein, at least one of R₂, R₅, R₆, or R₇ is a reactant group, an wherein R₂, R₅, R₆, or R₇ which are not a reactant group is a hydrogen atom or an alkyl, O-alkyl or O-aryl group, wherein R₄ is a hydrogen atom, an alkyl, O-alkyl or O-aryl group, a hydroxy group, or a phosphate group and wherein R_{3A} and R_{3B} are a hydrogen atom or an alkyl group.

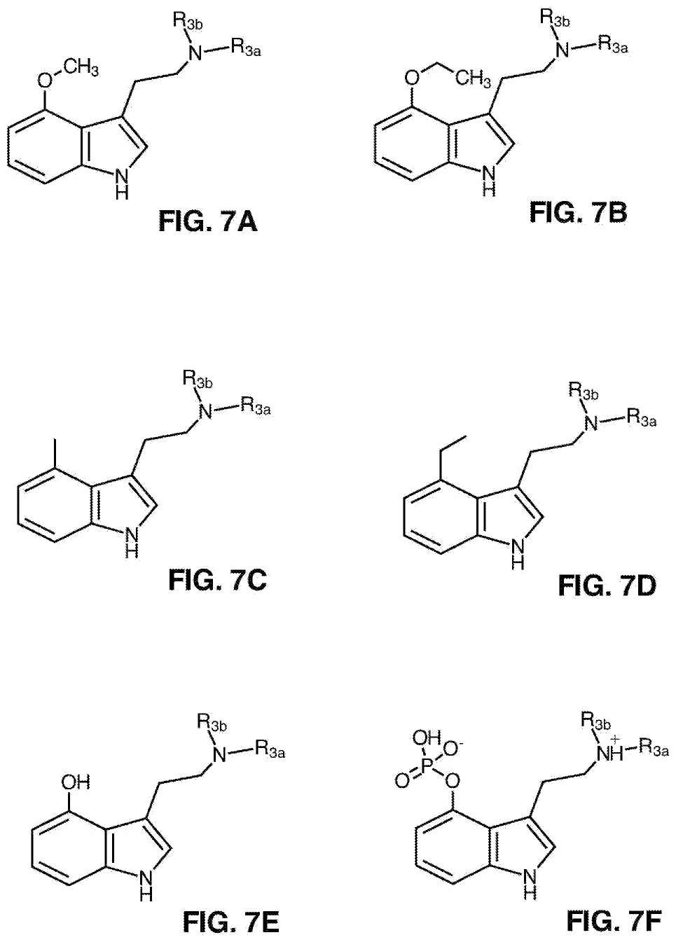

In one example embodiment, the reactant psilocybin derivative can be selected to be a chemical compound wherein R₄ is an O-alkyl group, R₂, R₅, R₆, and R₇ are a hydrogen atom, and R_{3A} and R_{3B} are a hydrogen atom, an alkyl group, an aryl group or an acyl group such as, for example, the reactant psilocybin derivative shown in FIGS. 7A and 7B.

In one example embodiment, the reactant psilocybin derivative can be selected to be a chemical compound wherein R₄ is an alkyl group, R₂, R₅, R₆, and R₇ are a hydrogen atom, and R_{3A} and R_{3B} are a hydrogen atom or an alkyl group, such as, for example, the reactant psilocybin derivative shown in FIGS. 7C and 7D.

In one example embodiment, the reactant psilocybin derivative can be selected to be a chemical compound wherein R₄ is a hydroxyl group, R₂, R₅, R₆, and R₇ are a hydrogen atom, and R_{3A} and R_{3B} are a hydrogen atom or an alkyl group, such as, for example, the reactant psilocybin derivative shown in FIG. 7E.

In one example embodiment, the reactant psilocybin derivative can be selected to be a chemical compound wherein R₄ is a phosphate group, R₂, R₅, R₆, and R₇ are a hydrogen atom, and R_{3A} and R_{3B} are a hydrogen atom or an alkyl group, such as, for example, the reactant psilocybin derivative shown in FIG. 7F.

The reactant psilocybin compound may be provided in a more or less chemically pure form, for example, in the form of a reactant psilocybin compound preparation having a purity of at least about 95%, at least about 96%, at least about 97%, at least about 98%, at least about 99%, or at least 99.9%. The reactant psilocybin may be chemically synthesized, or obtained from a fine chemical manufacturer.

The nitro group donating compound is in general any compound including a reactive nitro group. A particular suitable compound is nitric acid (HNO₃), or a nitrate salt in conjunction with another acid, a nitronium salt, such as nitronium tetraborofluoride, HNO₃ with acetic anhydride, or acyl nitrate such as acetyl nitrate, trifluomethansulfonyl nitrate, trifluoracetyl nitrate, nitric acid with a Lewis acid, such as copper (II) triflate etc.

The nitro group-containing compound may be provided in a more or less chemically pure form, for example, in the form of a nitro group-containing compound preparation having different purity, such as a dilute (1-30%) or concentrated (>30%) nitric acid, or even fuming nitric acid, or a nitrate salt with either a Bronsted-Lowry acid or a Lewis acid. The nitro group-containing compound may be chemically synthesized, or obtained from a fine chemical manufacturer.

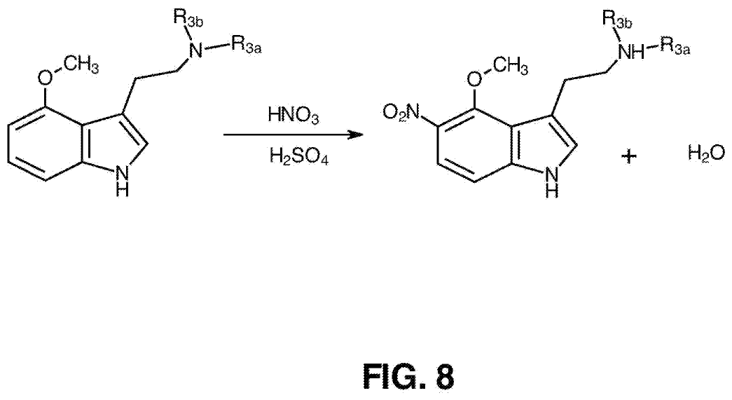

To further illustrate nitration reactions that may be performed according to the present disclosure, FIG. 8 shows an example chemical reaction wherein nitric acid is reacted with a 4-O-methyl psilocybin derivative in a chemical reaction which results in the formation of a 4-O-methyl-5-nitro-psilocybin derivative.

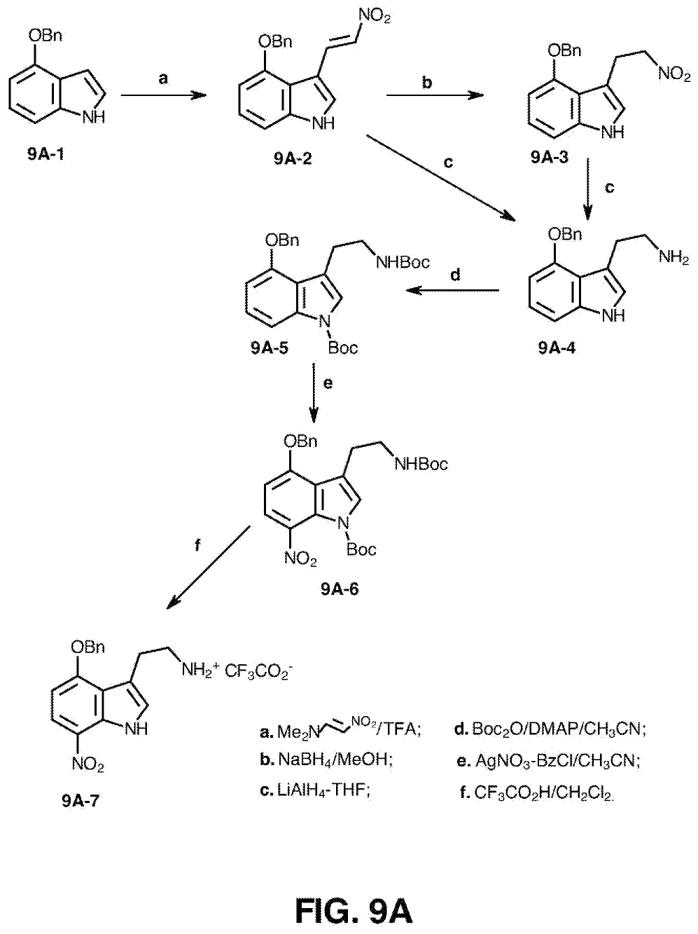

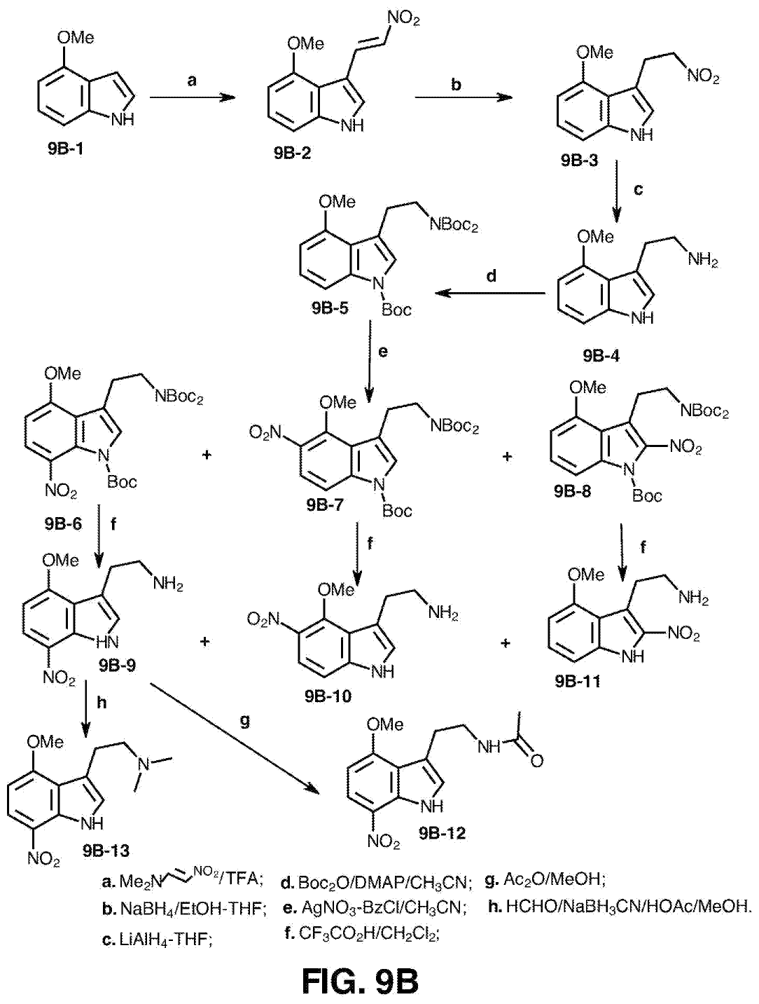

Referring now to FIG. 9A, shown therein is a further example of a chemical synthesis process resulting in the formation of a nitrated psilocybin derivative, notably a 4-benzyloxy-7-nitro psilocybin derivative 9A-7 which can be initiated starting with 4-benzyloxyindole (9A-1). The process can be initiated by an acid-catalyzed regioselective 3-nitrovinylation of compound 9A-1 by reacting compound 9A-1 with 1-(dimethylamino)-2-nitroethylene and using trifluoroacetic acid as an activator. This can provide the desired (E)-4-benzyloxy-3-(2-nitrovinyl)indole (9A-2). The alkene functionality of compound 9A-2 can subsequently be reduced using sodium borohydride as the reagent to provide the 4-benzyloxy-3-(2-nitroethyl)indole (9A-3). A further reduction of the nitro functionality using lithium aluminum hydride can furnish the desired 4-benzyloxy psilocybin derivative (9A-4). Alternatively, the 2-nitrovinyl functionality in compound 9A-2 can be directly reduced to 2-aminoethyl group in compound 9A-4 using lithium aluminum hydride. To facilitate a regioselective nitration, both the primary amine and indole N—H functionalities can be protected with tert-butoxycarbonyl groups using an excess amount of di-tert-butyl dicarbonate and 4-N,N-dimethylaminopyridine, to provide the corresponding N1,N-di-Boc protected derivative 9A-5. A subsequent treatment of compound 9A-5 with benzoyl nitrate, can generate in-situ by reacting silver nitrate with benzoyl chloride in anhydrous acetonitrile to provide the C₇-mononitrated compound 9A-6 as a major constituent Finally, a full removal of the two Boc protecting groups of compound 9A-6 by reacting with trifluoroacetic acid can afford the desired 4-benzyloxy-7-nitro psilocybin derivative, isolated in the form of trifluoroacetic acid salt (9A-7).

Referring now to FIG. 9B, shown therein is a further example of a chemical synthesis process resulting in the formation of a nitrated psilocybin derivative, notably a nitrated 4-methoxy psilocybin derivatives respectively at C₇ (9B-8, 9-B10 and 9-B11) and C₅ (9B-9), which are synthesized from 4-methoxyindole (9B-1). The synthesis process can begin with an acid-catalyzed regioselective 3-nitrovinylation of compound 9B-1 by reacting compound 9B-1 with 1-(dimethylamino)-2-nitroethylene and using trifluoroacetic acid as an activator. This can provide the desired (E)-4-methoxy-3-(2-nitrovinyl)indole (9B-2). The alkene functionality of compound 9B-2 can subsequently be reduced using sodium borohydride as the reagent to provide the reduced 4-methoxy-3-(2-nitroethyl)indole (9B-3). A further reduction of the nitro functionality using either lithium aluminum hydride in tetrahydrofuran or ammonium formate in methanol at 60° C. in the presence of 10% palladium on charcoal can furnish the desired 4-methoxy psilocybin derivative (9B-4) as a key intermediate. To facilitate a regioselective nitration, both the primary amine and indole N—H functionalities can be protected with tert-butoxycarbonyl groups using an excess amount of di-tert-butyl dicarbonate and 4-N,N-dimethylaminopyridine, to provide the corresponding N1,N,N-tri-Boc protected derivative 9B-5. A subsequent treatment of the fully protected 4-methoxy psilocybin derivative (9B-5) with benzoyl nitrate, can be generated in-situ by reacting silver nitrate with benzoyl chloride in anhydrous dichloromethane to provide two partially mononitrated isomers that have a nitro group at C₇ (compound 9B-6) and C₅ (compound 9B-7) and C₂ (compound 9B-8). Finally, a full removal of the three Boc protecting groups in compound 9B-6 by reacting with trifluoroacetic acid can afford the desired 4-methoxy-7-nitro psilocybin derivative, isolated in the form of trifluoroacetic acid salt (9B-9). In a similar manner, the treatment of either compounds 9B-7 and 984 with trifluoroacetic acid can yield the desired 4-methoxy-5-nitro psilocybin derivative (9B-10) and 4-methoxy-2-nitro psilocybin derivative (9B-11). Furthermore, using the 4-methoxy-7-nitro psilocybin derivative (9B-9) as a substrate, other modifications can be carried out. For example, a regioselective acylation can be conducted on the side chain primary amine using acetic anhydride as a reagent to afford the desired N-acetylated 4-methoxy-7-nitro psilocybin derivative (9B-12), and a reductive amination can also be carried out on the same primary amine on the side chain by reacting with formaldehyde and sodium cyanoborohydride to obtain the N,N-dimethylated 4-methoxy-7-nitro psilocybin (9B-13).

Thus, it is noted that the reactions depicted in FIGS. 9A and 9B show a reaction sequences starting from the 4-alkoxyindole resulting in the partially nitrated psilocybin products. Other nitrated psilocybin derivatives can be prepared by following a similar reaction sequence reaction using a 4-alkoxyindole derivative that contains one or more compatible substituent(s) on the ring, such as alkyl, halides etc. The amount of formaldehyde in the final reductive amination step can be reduced to allow mono N-alkylation, and the formaldehyde can be switched to any other aldehyde/ketone to obtain variants of substituents on the nitrogen. The amine functionality can also be N-alkylated using an appropriate alkylating reagent such as an alkyl halide, alkyl p-tosylate/mesylate/triflate, or conjugated reagents such as α,β-unsaturated ester/amide/aldehyde/ketone (Michael additions) to afford higher substituted amines or quaternary ammonium salts. Multiple N-alkylations can be carried out in one-pot or stepwise manner using the same or different alkyl halides. The amine functionality can also be N-acylated using an appropriate acylating reagent such as an acid anhydride or acyl halide.

Thus, it will now be clear that, in an aspect hereof, other nitrated psilocybin derivatives can be formed by following a similar reaction sequence reaction using an indole derivative that contains one or more alkyl, alkoxy, acyloxy groups or halogen group on the indole ring other than the C-3 position, as a starting material. Once the 2-aminoethyl chain is introduced, the primary amine group of the side chain can be protected alone or protected along with the indole N—H group using Boc or other appropriate protecting groups for facilitate the subsequent nitration(s). The primary amine on the 2-aminoethyl group can be further modified with aldehyde/ketone using reductive amination conditions either in one-pot, or stepwise manner for afford N-monoalkylated- or N,N-dialkylated products. Instead of using reductive amination, the amine functionality can also be N-alkylated using an appropriate alkylating reagent such as an alkyl halides, alkyl p-tosylate/mesylate/triflate or/conjugated reagents to afford higher substituted amines or quaternary ammonium salts. The amine functionality can also be N-acylated using an appropriate acylating reagent such as an acid anhydride or acyl halide.

In general, the reactants are reacted under reaction conditions which permit the reactants to chemically react with each other and form a product, i.e. the nitrated psilocybin derivatives of the present disclosure. Such reactions conditions may be selected, adjusted and optimized as known by those of skill in the art. Thus, for example, the reaction may be catalyzed by, for example, sulfuric acid (H₂SO₄) (see: FIG. 8). Other catalysts that may be used include HNO₃ with acetic acid (AcOH); HNO₃ with acetic anhydride, trifluromethansulfonyl nitrate, trifluoracetyl nitrate, HNO₃ and NaNO₂ with AcOH: HNO₃ with CH₂Cl₂; HNO₃ and NaNO₂ with CHCl₃; HNO₃ and NaNO₂ with CH₂Cl₂; and NH₂CONH₂ HNO₃, or a solid catalyst such as claycop (Gigante et al., J. Org. Chem., 1995, (60), 3445-3447), or a phase catalyst such as tetra-n-butylammonium bromide (Joshi et al, Org. Proc. Res. Dev. 2003, 7 (1), 95-97).

Furthermore, it is noted that the performance of the reactions, in example different embodiments, may involve nitration of different carbon atoms, i.e. the C₂, C₅, C₆ and/or C₇ atom. In general reaction conditions may be selected so that different carbon atoms or combinations thereof are nitrated. Thus, for example, the nitration of a 4-O-substituted psilocin derivative would regioselectively install one nitro group at C₅, C₆ or C₇, or two nitro groups at both C₅ and C₇ positions.

The reactions may be conducted in any suitable reaction vessel (e.g. a tube, bottle). Suitable solvents that may be used are for example water, acetic acid, dichloromethane, chloroform, 1,2-dichloroethane, nitrobenzene etc. Suitable temperatures may range from, for example, e.g. from about −78° C. to about 60° C. Furthermore, reaction times may be varied. As will readily be appreciated by those of skill in the art, the reaction conditions may be optimized, for example by preparing several psilocybin derivative reactants preparations and nitro group donating compounds and reacting these in different reaction vessels under different reaction conditions, for example, at different temperatures, using different solvents, using different catalysts etc., evaluating the obtained nitrated psilocybin derivative reaction product, adjusting reaction conditions, and selecting a desired reaction condition. Further general guidance regarding appropriate reaction conditions for performing nitration reactions may be found in, for example “Nitration: Methods and Mechanisms”, by Olah, G. A.; Malhotra, R.; Narang, S. C. John Wiley & Sons Inc. 1989.

In another aspect of the present disclosure, the nitrated psilocybin compounds may be made biosynthetically. Accordingly, the present disclosure further includes, in one embodiment, a method of making a nitrated psilocybin derivative the method comprising:
(a) contacting a nitrated psilocybin precursor compound with a host cell comprising a psilocybin biosynthetic enzyme complement; and
(b) growing the host cell to produce a nitrated psilocybin derivative or salts thereof having the formula (I):

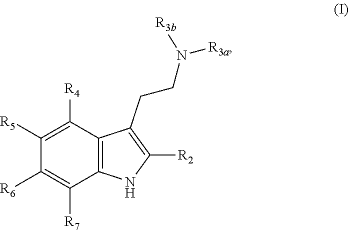

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein R_{3A} and R_{3B} are a hydrogen atom, an alkyl group, an aryl group, or an acyl group.

Implementation of the foregoing example embodiment initially involves providing nitrated psilocybin precursor compounds and host cells having a psilocybin biosynthetic enzyme complement. Accordingly, next, example nitrated psilocybin precursor compounds and example host cells that may be selected and used in accordance with the present disclosure will be described. Thereafter, example methodologies and techniques will be described to contact and use the nitrated psilocybin precursor compounds and cells to produce example nitrated psilocybin compounds.

A variety of nitrated psilocybin precursor compounds may be selected, prepared and used. In some embodiments, for example, the nitrated psilocybin precursor compound is a compound comprising a nitrated indole prototype structure. Examples of such compounds are a nitrated indole, e.g. 2-nitro-indole, 4, nitro-indole, 5-nitro-indole, 6-nitro-indole and 7-nitro indole; and nitrated tryptophan derivatives, e.g. 2-nitro-tryptophan, 4, nitro-tryptophan, 5-nitro-tryptophan, 6-nitro-tryptophan and 7-nitro tryptophan.

Further nitrated psilocybin precursor compounds that may be used include nitrated indoles, having the formula (XXVI):

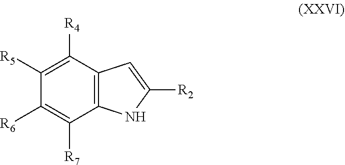

wherein at least one of R₂, R₄, R₅, R₆ and R₇ is a nitro group, wherein R₂, R₄, R₅, R₆ and R₇ when they are not nitrated are hydrogen atoms, or an alkyl, O-alkyl or O-aryl group, wherein R₄ when it is not nitrated is a hydrogen atom, an alkyl, O-alkyl or O-aryl group, a hydroxy group, or a phosphate group.

Further nitrated psilocybin precursor compounds that may be used include compounds having the formula (XXIV):

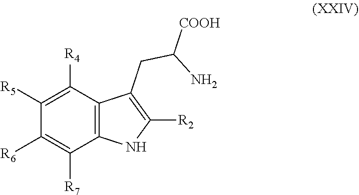

wherein at least one of R₂, R₄, R₅, R₆ and R₇ is a nitro group, wherein R₂, R₄, R₅, R₆ and R₇ when they are not nitrated are hydrogen atoms, or an alkyl, O-alkyl or O-aryl group, wherein R₄ when it is not nitrated is a hydrogen atom, an alkyl, O-alkyl or O-aryl group, a hydroxy group, or a phosphate group

Turning now to the host cells that can be used in accordance with the present disclosure, it is initially noted that a variety of host cells may be selected in accordance with the present disclosure, including microorganism host cells, plant host cells, and animal host cells.

In accordance herewith the host cell includes a psilocybin biosynthetic enzyme complement. Such cells can be obtained in at least two ways. First, in some embodiments, host cells may be selected in which a psilocybin biosynthetic enzyme complement is naturally present Generally cells naturally producing psilocybin for example, cells of fungal species belonging to the genus psilocybe, are suitable in this respect. Second, in some embodiments, a host cell that not naturally produces psilocybin may be modulated to produce a psilocybin biosynthetic enzyme complement. Thus, for example, a nucleic acid sequence encoding a psilocybin biosynthetic enzyme complement may be introduced into a host cell, and upon cell growth the host cells can make the psilocybin biosynthetic enzyme complement.

Typically a nucleic acid sequence encoding one or more enzymes constituting a psilocybin biosynthetic enzyme complement further includes one or more additional nucleic acid sequences, for example, a nucleic acid sequences controlling expression of the one or more enzymes, and these one or more additional nucleic acid sequences together with the nucleic acid sequence encoding the one or more enzymes can be said to form a chimeric nucleic acid sequence.

A host cell which upon cultivation expresses the chimeric nucleic acid can be selected and used in accordance with the present disclosure. Suitable host cells in this respect include, for example, microbial cells, such as bacterial cells, yeast cells, for example, and algal cells or plant cells. A variety of techniques and methodologies to manipulate host cells to introduce nucleic acid sequences in cells and attain expression exists and are well known to the skilled artisan. These methods include, for example, cation based methods, for example, lithium ion or calcium ion based methods, electroporation, biolistics, and glass beads based methods. As will be known to those of skill in the art, depending on the host cell selected, the methodology to introduce nucleic acid material in the host cell may vary, and, furthermore, methodologies may be optimized for uptake of nucleic acid material by the host cell, for example, by comparing uptake of nucleic acid material using different conditions. Detailed guidance can be found, for example, in Sambrook et al., Molecular Cloning, a Laboratory Manual, Cold Spring Harbor Laboratory Press, 2012, Fourth Ed. It is noted that the chimeric nucleic acid is a non-naturally occurring chimeric nucleic acid sequence and can be said to be heterologous to the host cell.

In some embodiments, the one or more enzymes constituting a psilocybin enzyme complement can be selected from by a nucleic acid sequence selected from the nucleic acid sequences consisting of:
(a) SEQ.ID NO: 4, SEQ.ID NO: 6, and SEQ.ID NO: 11;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ.ID NO: 5, SEQ.ID NO: 7, and SEQ.ID NO: 12;
(f) a nucleic acid sequence that encodes a functional variant of any one of the amino acid sequences set forth in SEQ.ID NO: 5, SEQ.ID NO: 7, and SEQ.ID NO: 12; and
(g) a nucleic acid sequence that hybridizes under stringent conditions to any one of the nucleic acid sequences set forth in (a), (b), (c), (d), (e) or (f).

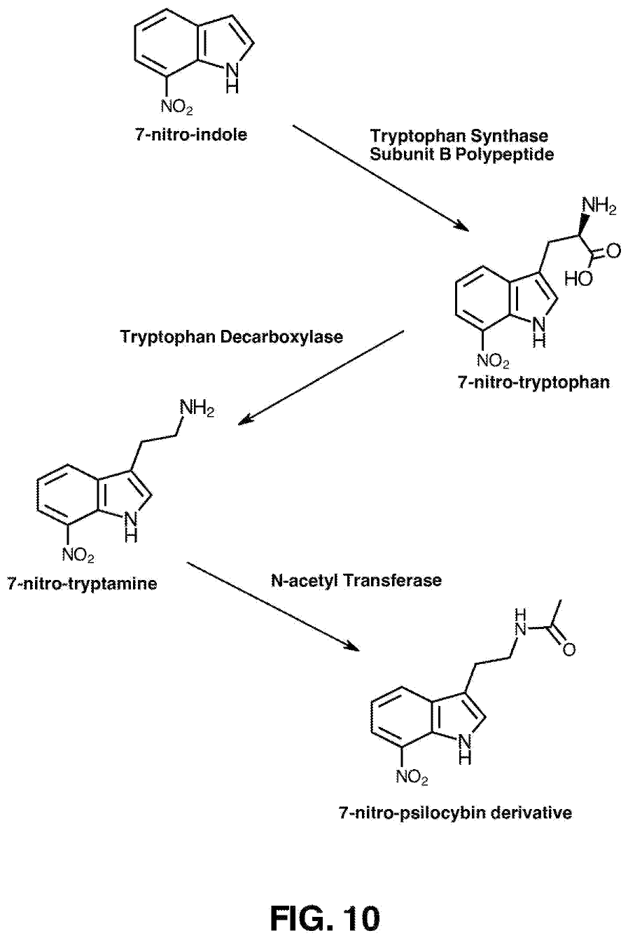

Thus any of the nucleic acid sequence set forth in (a), (b), (c), (d), (e), (f) or (g) may be selected and introduced into a host cell. In particular, however the nucleic acid sequence is selected in conjunction with the selected psilocybin precursor compound, as hereinafter further discussed in reference with FIG. 10.

One example host cell that conveniently may be used is Escherichia coli. The preparation of the E. coli vectors may be accomplished using commonly known techniques such as restriction digestion, ligation, gel electrophoresis, DNA sequencing, the polymerase chain reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Suitable promoter sequences for use in E. coli include, for example, the T7 promoter, the T5 promoter, tryptophan (trp) promoter, lactose (lac) promoter, tryptophan/lactose (tac) promoter, lipoprotein (Ipp) promoter, and A phage PL promoter. Typically, cloning vectors contain a marker, for example, an antibiotic resistance marker, such as ampicillin or kanamycin resistance marker, allowing selection of transformed cells. Nucleic acid sequences may be introduced in these vectors, and the vectors may be introduced in E. coli by preparing competent cells, electroporation or using other well known methodologies to a person of skill in the art. E. coli may be grown in an appropriate medium, such as Luria-Broth medium and harvested. Recombinant expression vectors may readily be recovered from cells upon harvesting and lysing of the cells.

Another example host cell that may be conveniently used is a yeast cell. Example yeast host cells that can be used are yeast cells belonging to the genus Candida, Kluyveromyces, Saccharomyces. Schizosaccharomyces, Pichia, Hansenula, and Yarrowia. In specific example embodiments, the yeast cell can be a Saccharomyces cerevisiae cell, a Yarrowia lipolytica cell, or Pichia pastoris cell.

A number of vectors exist for the expression of recombinant proteins in yeast host cells. Examples of vectors that may be used in yeast host cells include, for example, Yip type vectors, YEp type vectors, YRp type vectors, YCp type vectors, pGPD-2, pAO815, pGAPZ, pGAPZα, pHIL-D2, pHIL-S1, pPIC3.5K, pPIC9K, pPICZ, pPICZα, pPIC3K, pHWO10, pPUZZLE and 2 μm plasmids. Such vectors are known to the art and are, for example, described in Cregg et al., Mol Biotechnol. (2000) 16(1): 23-52. Suitable promoter sequences for use in yeast host cells are also known and described, for example, in Mattanovich et al., Methods Mol. Biol., 2012, 824:329-58, and in Romanos et al., 1992, Yeast 8: 423-488. Examples of suitable promoters for use in yeast host cells include promoters of glycolytic enzymes, like triosephosphate isomerase (TPI), phosphoglycerate kinase (PGK), glyceraldehyde-3-phosphate dehydrogenase (GAPDH or GAP) and variants thereof, lactase (LAC) and galactosidase (GAL), P. pastoris glucose-6-phosphate isomerase promoter (PPGI), the 3-phosphoglycerate kinase promoter (PPGK), the glycerol aldehyde phosphate dehydrogenase promoter (PGAP), translation elongation factor promoter (PTEF), S. cerevisiae enolase (ENO-1), S. cerevisiae galactokinase (GAL1), S. cerevisiae alcohol dehydrogenase/glyceraldehyde-3-phosphate dehydrogenase (ADH1, ADH2/GAP), S. cerevisiae triose phosphate isomerase (TPI), S. cerevisiae metallothionein (CUP1), and S. cerevisiae 3-phosphoglycerate kinase (PGK), and the maltase gene promoter (MAL). Marker genes suitable for use in yeast host cells are also known to the art. Thus, antibiotic resistance markers, such as ampicillin resistance markers, can be used in yeast, as well as marker genes providing genetic functions for essential nutrients, for example, leucine (LEU2), tryptophan (TRP1 and TRP2), uracil (URA3, URA5, URA6), histidine (HIS3), and the like. Methods for introducing vectors into yeast host cells can, for example, be found in S. Kawai et al., 2010. Bioeng. Bugs 1(6): 395-403.

Further, guidance with respect to the preparation of expression vectors and introduction thereof into host cells, including in E. coli cells, yeast cells, and other host cells, may be found in, for example: Sambrook et al., Molecular Cloning, a Laboratory Manual, Cold Spring Harbor Laboratory Press, 2012, Fourth Ed.

Thus, to briefly recap, a host cell comprising a chimeric nucleic acid comprising (i) a nucleic acid sequence controlling expression in a host cell and (ii) a nucleic acid sequence encoding a psilocybin biosynthetic enzyme complement, can be prepared in accordance with the present disclosure.

In accordance herewith, host cells are grown to multiply and to express a chimeric nucleic acid. Expression of the chimeric nucleic acid results in the biosynthetic production in the host cell of a psilocybin biosynthetic enzyme complement. Growth media and growth conditions can vary depending on the host cell that is selected, as will be readily appreciated to those of ordinary skill in the art. Growth media typically contain a carbon source, one or several nitrogen sources, essential salts including salts of potassium, sodium, magnesium, phosphate and sulphate, trace metals, water soluble vitamins, and process aids including but not limited to antifoam agents, protease inhibitors, stabilizers, ligands and inducers. Example carbon sources are e.g. mono- or disaccharides. Example nitrogen sources are, e.g. ammonia, urea, amino acids, yeast extract, corn steep liquor and fully or partially hydrolyzed proteins. Example trace metals are e.g. Fe, Zn, Mn, Cu, Mo and H₃BO₃. Example water soluble vitamins are e.g. biotin, pantothenate, niacin, thiamine. p-aminobenzoic acid, choline, pyridoxine, folic acid, riboflavin and ascorbic acid. Further, specific example media include liquid culture media for the growth of yeast cells and bacterial cells including, Luria-Bertani (LB) broth for bacterial cell cultivation, and yeast extract peptone dextrose (YEPD or YPD), for yeast cell cultivation. Further media and growth conditions can be found in Sambrook et al., Molecular Cloning, a Laboratory Manual, Cold Spring Harbor Laboratory Press, 2012, Fourth Ed.

In order for the host cells to produce the nitrated psilocybin compound, the cells are provided with a psilocybin precursor compound. Thus in accordance herewith, host cells may be contacted with a psilocybin precursor compound. In some embodiments, a psilocybin precursor compound can be exogenously supplied, for example, by including a psilocybin precursor compound in the growth medium of the host cells, and growing the host cells in a medium including the psilocybin precursor compound.

Referring next to FIG. 10, shown therein is an example biosynthetic pathway showing the conversion of example psilocybin precursor compounds to form a nitrated psilocybin. Thus, as can be appreciated from FIG. 10, various psilocybin precursor compounds may be selected and prepared in nitrated form, in conjunction with a psilocybin biosynthetic enzyme complement. Thus, by way of example, nitrated tryptophan (e.g. 2-, 5-, 6-, or 7-nitrated tryptophan) may be selected and contacted with a host cell comprising a psilocybin biosynthetic enzyme complement comprising tryptophan decarboxylase and optionally N-acetyl transferase, and upon growth of the cells nitrated psilocybin derivatives can be formed. By way of further example, nitrated indole (e.g. 2-, 5-, 6-, or 7-nitrated indole) may be selected and contacted with a host cell comprising a psilocybin biosynthetic enzyme complement comprising tryptophan synthase subunit B polypeptide and tryptophan decarboxylase and optionally N-acetyl transferase, and upon growth of the cells nitrated psilocybin derivatives can be formed

In some embodiments, the psilocybin precursor compound can be a nitrated psilocybin precursor compound which is exogenously supplied to a host cell, for example by inclusion in the host cell's growth medium. Thus, for example, referring to FIG. 10, it will be understood that in accordance herewith, for example, 7-nitro-indole or 7-nitro-tryptophan, may be included in the growth medium of a host cell comprising a psilocybin biosynthetic enzyme complement.

Referring to FIG. 10, in a further example embodiment, the nitrated psilocybin precursor compound can be a nitrated indole, having the formula (XXVI):

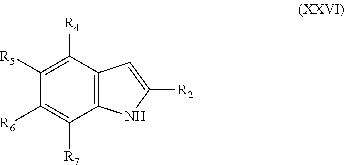

wherein at least one of R₂, R₄, R₅, R₆ and R₇ is a nitro group, wherein R₂, R₄, R₅, R₆ and R₇ when they are not nitrated are hydrogen atoms, or an alkyl, O-alkyl or O-aryl group, wherein R₄ when it is not nitrated is a hydrogen atom, an alkyl, O-alkyl or O-aryl group, a hydroxy group, or a phosphate group;
   the psilocybin biosynthetic enzyme complement can comprise:
   (i) a tryptophan synthase subunit B polypeptide encoded by a nucleic acid selected from:
(a) SEQ.ID NO: 6;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having any one of the amino acid sequences set forth in SEQ.ID NO: 7;
(f) a nucleic acid sequence that encodes a functional variant of any one of the amino acid sequences set forth in SEQ.ID NO: 7; and
(g) a nucleic acid sequence that hybridizes under stringent conditions to any one of the nucleic acid sequences set forth in (a), (b), (c), (d), (e) or (f); and
   (ii) a tryptophan decarboxylase encoded by a nucleic acid sequence selected from:
(a) SEQ.ID NO: 11;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having any one of the amino acid sequences set forth in SEQ.ID NO: 12;
(f) a nucleic acid sequence that encodes a functional variant of any one of the amino acid sequences set forth in SEQ.ID NO: 12; and
(g) a nucleic acid sequence that hybridizes under stringent conditions to any one of the nucleic acid sequences set forth in (a), (b), (c), (d), (e) or (f); and the formed nitrated psilocybin derivative can be a compound having formula (XXV):

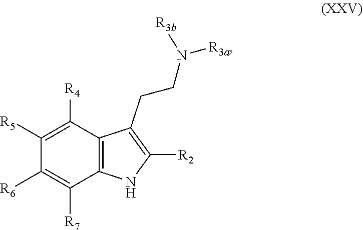

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein at least one of R_{3A} and R_{3B} are hydrogen atom.

Referring further to FIG. 10, in another example embodiment, the nitrated psilocybin precursor compound can be a compound, having the formula (XXIV):

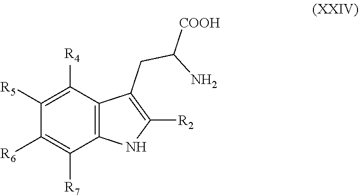

wherein at least one of R₂, R₄, R₅, R₆ and R₇ is a nitro group, wherein R₂, R₄, R₅, R₆ and R₇ when they are not nitrated are hydrogen atoms, or an alkyl, O-alkyl or O-aryl group, wherein R₄ when it is not nitrated is a hydrogen atom, an alkyl, O-alkyl or O-aryl group, a hydroxy group, or a phosphate group;
   the psilocybin biosynthetic enzyme complement can comprise:
   a tryptophan decarboxylase encoded by a nucleic acid sequence selected from:
(a) SEQ.ID NO: 11;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having any one of the amino acid sequences set forth in SEQ.ID NO: 12;
(f) a nucleic acid sequence that encodes a functional variant of any one of the amino acid sequences set forth in SEQ.ID NO: 12; and
(g) a nucleic acid sequence that hybridizes under stringent conditions to any one of the nucleic acid sequences set forth in (a), (b), (c), (d), (e) or (f); and the formed nitrated psilocybin derivative can be a compound having formula (XXV):

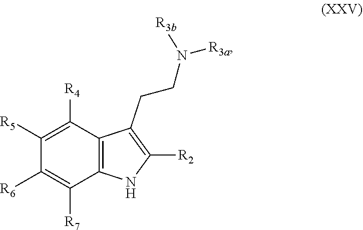

wherein at least one of R₂, R₄, R₅, R₆, or R₇ is a nitro group, and wherein each non-nitrated R₂, R₅, R₆, or R₇ is a hydrogen atom or an alkyl, O-alkyl or O-aryl group, wherein R₄ when it is not nitrated is a hydrogen atom, an alkyl, O-alkyl or O-aryl group, a hydroxy group, or a phosphate group, and wherein at least one of R_{3A} and R_{3B} are hydrogen atom.

In some embodiments, in formula (XXV) R_{3A} and R_{3B} are each a hydrogen atom.

Referring again to FIG. 10, the psilocybin biosynthetic enzyme complement can, in addition to the aforementioned tryptophan decarboxylase and tryptophan synthase subunit B polypeptide further comprise an N-acetyl transferase.

In at least one embodiment, in an aspect, the N-acetyl transferase can be an enzyme encoded by. a nucleic acid sequence selected from:
(a) SEQ.ID NO: 4;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having any one of the amino acid sequences set forth in SEQ.ID NO: 5;
(f) a nucleic acid sequence that encodes a functional variant of any one of the amino acid sequences set forth in SEQ.ID NO: 5; and
(g) a nucleic acid sequence that hybridizes under stringent conditions to any one of the nucleic acid sequences set forth in (a), (b), (c), (d), (e) or (f).

In at least one embodiment, in an aspect, the formed nitrogenated psilocybin compound can have the formula (XXVII):

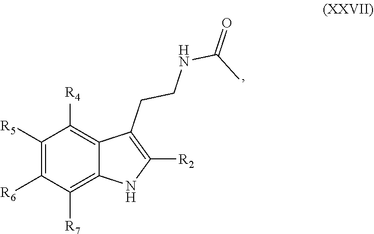

wherein, at least one of R₂, R₄, R₅, R₆ or R₇ is a nitro group, wherein each non-nitrated R₂, R₅, R₆, or R₇ is a hydrogen atom or an alkyl group or O-alkyl group, wherein R₄ when it is not nitrated is a phosphate group, a hydrogen atom or an alkyl group or O-alkyl group.

It will be clear to those of skill in the art that a significant variety of different nitrated psilocybin precursor compounds may be selected. FIG. 10 in this respect provides guidance and allows a person of skill in the art to select appropriate psilocybin precursor compounds and a matching a psilocybin biosynthetic enzyme complement.

Upon production by the host cells of the nitrated psilocybin compounds in accordance with the methods of the present disclosure, the nitrated psilocybin compounds may be extracted from the host cell suspension, and separated from other constituents within the host cell suspension, such as media constituents and cellular debris. Separation techniques will be known to those of skill in the art and include, for example, solvent extraction (e.g. butane, chloroform, ethanol), column chromatography based techniques, high-performance liquid chromatography (HPLC), for example, and/or countercurrent separation (CCS) based systems. The recovered nitrated psilocybin compounds may be obtained in a more or less pure form, for example, a preparation of nitrated psilocybin compounds of at least about 60% (w/w), about 70% (w/w), about 80% (w/w), about 90% (w/w), about 95% (w/w), about 96% (w/w), about 97% (w/w), about 98% (w/w) or about 99% (w/w) purity may be obtained. Thus, in this manner, nitrated psilocybin derivatives in more or less pure form may be prepared.

Similarly, other methods of making the nitrated psilocybin compounds that may be used in accordance herewith may yield preparations of nitrated compounds of at least about 60% (w/w), about 70% (w/w), about 80% (w/w), about 90% (w/w), about 95% (w/w), about 96% (w/w), about 97% (w/w), about 98% (w/w), or about 99% (w/w) purity.

It will now be clear form the foregoing that novel nitrated psilocybin derivatives are disclosed herein. The nitrated psilocybin compounds may be formulated for use as a pharmaceutical drug or recreational drug. The nitrated psilocybin compounds may also be used as a feedstock to produce other psilocybin derivatives.

Hereinafter are provided examples of specific implementations for performing the methods of the present disclosure, as well as implementations representing the compositions of the present disclosure. The examples are provided for illustrative purposes only, and are not intended to limit the scope of the present disclosure in any way.

### SUMMARY OF SEQUENCES

SEQ.ID NO: 1 sets forth a nucleic acid sequence of pCDM4 vector

SEQ.ID NO: 2 sets forth a nucleic acid sequence encoding a synthetic FLAG epitope tag polypeptide

SEQ.ID NO: 3 sets forth deduced amino acid sequence of a synthetic FLAG epitope tag polypeptide

SEQ.ID NO: 4 sets forth a nucleic acid sequence encoding a Streptomyces griseofuscus PsmF N-acetyltransferase polypeptide.

SEQ.ID NO: 5 sets forth a deduced amino acid sequence of a Streptomyces griseofuscus PsmF N-acetyltransferase polypeptide.

SEQ.ID NO: 6 sets forth a nucleic acid sequence encoding a mutated Thermotoga maritima TmTrpB-2F3 tryptophan synthase subunit B polypeptide.

SEQ.ID NO: 7 sets forth a deduced amino acid sequence of a mutated Thermotoga maritima TmTrpB-2F3 tryptophan synthase subunit B polypeptide.

SEQ.ID NO: 8 sets forth a nucleic acid sequence encoding a synthetic V5 epitope tag polypeptide

SEQ.ID NO: 9 sets forth deduced amino acid sequence of a synthetic V5 epitope tag polypeptide

SEQ.ID NO: 10 sets forth a nucleic acid sequence of pETM6-H10 vector

SEQ.ID NO: 11 sets forth a nucleic acid sequence encoding a Bacillus atrophaeus BaTDC tryptophan decarboxylase polypeptide.

SEQ.ID NO: 12 sets forth a deduced amino acid sequence of a Bacillus atrophaeus BaTDC tryptophan decarboxylase polypeptide.

SEQUENCE LISTING
SEQ.ID NO: 1
GCTAACAGCGCGATTTGCTGGTGACCCAATGCGACCAGATGCTCCACGCCCAGTCGCGT
ACCGTCTTCATGGGAGAAAATAATACTGTTGATGGGTGTCTGGTCAGAGACATCAAGAA
ATAACGCCGGAACATTAGTGCAGGCAGCTTCCACAGCAATGGCATCCTGGTCATCCAGC
GGATAGTTAATGATCAGCCCACTGACGCGTTGCGCGAGAAGATTGTGCACCGCCGCTTT
ACAGGCTTCGACGCCGCTTCGTTCTACCATCGACACCACCACGCTGGCACCCAGTTGAT
CGGCGCGAGATTTAATCGCCGCGACAATTTGCGACGGCGCGTGCAGGGCCAGACTGGAG
GTGGCAACGCCAATCAGCAACGACTGTTTGCCCGCCAGTTGTTGTGCCACGCGGTTGGG
AATGTAATTCAGCTCCGCCATCGCCGCTTCCACTTTTTCCCGCGTTTTCGCAGAAACGT
GGCTGGCCTGGTTCACCACGCGGGAAACGGTCTGATAAGAGACACCGGCATACTCTGCG
ACATCGTATAACGTTACTGGTTTCACATTCACCACCCTGAATTGACTCTCTTCCGGGCG
CTATCATGCCATACCGCGAAAGGTTTTGCGCCATTCGATGGTGTCCGGGATCTCGACGC
TCTCCCTTATGCGACTCCTGCATTAGGAAGCAGCCCAGTAGTAGGTTGAGGCCGTTGAG
CACCGCCGCCGCAAGGAATGGTGCATGCAAGGAGATGGCGCCCAACAGTCCCCCGGCCA
CGGGGCCTGCCACCATACCCACGCCGAAACAAGCGCTCATGAGCCCGAAGTGGCGAGCC
CGATCTTCCCCATCGGTGATGTCGGCGATATAGGCGCCAGCAACCGCACCTGTGGCGCC
GGTGATGCCGGCCACGATGCGTCCGGCGTAGCCTAGGATCGAGATCGATCTCGATCCCG
CGAAATTAATACGACTCACTATAGGGGAATTGTGAGCGGATAACAATTCCCCTCTAGAA
ATAATTTTGTTTAACTTTAAGAAGGAGATATACATATGGCAGATCTCAATTGGATATCG
GCCGGCCACGCGATCGCTGACGTCGGTACCCTCGAGTCTGGTAAAGAAACCGCTGCTGC
GAAATTTGAACGCCAGCACATGGACTCGTCTACTAGTCGCAGCTTAATTAACCTAAACT
GCTGCCACCGCTGAGCAATAACTAGCATAACCCCTTGGGGCCTCTAAACGGGTCTTGAG
GGGTTTTTTGCTAGCGAAAGGAGGAGTCGACACTGCTTCCGGTAGTCAATAAACCGGTA
AACCAGCAATAGACATAAGCGGCTATTTAACGACCCTGCCCTGAACCGACGACCGGGTC
ATCGTGGCCGGATCTTGCGGCCCCTCGGCTTGAACGAATTGTTAGACATTATTTGCCGA
CTACCTTGGTGATCTCGCCTTTCACGTAGTGGACAAATTCTTCCAACTGATCTGCGCGC
GAGGCCAAGCGATCTTCTTCTTGTCCAAGATAAGCCTGTCTAGCTTCAAGTATGACGGG
CTGATACTGGGCCGGCAGGCGCTCCATTGCCCAGTCGGCAGCGACATCCTTCGGCGCGA
TTTTGCCGGTTACTGCGCTGTACCAAATGCGGGACAACGTAAGCACTACATTTCGCTCA
TCGCCAGCCCAGTCGGGCGGCGAGTTCCATAGCGTTAAGGTTTCATTTAGCGCCTCAAA
TAGATCCTGTTCAGGAACCGGATCAAAGAGTTCCTCCGCCGCTGGACCTACCAAGGCAA
CGCTATGTTCTCTTGCTTTTGTCAGCAAGATAGCCAGATCAATGTCGATCGTGGCTGGC
TCGAAGATACCTGCAAGAATGTCATTGCGCTGCCATTCTCCAAATTGCAGTTCGCGCTT
AGCTGGATAACGCCACGGAATGATGTCGTCGTGCACAACAATGGTGACTTCTACAGCGC
GGAGAATCTCGCTCTCTCCAGGGGAAGCCGAAGTTTCCAAAAGGTCGTTGATCAAAGCT
CGCCGCGTTGTTTCATCAAGCCTTACGGTCACCGTAACCAGCAAATCAATATCACTGTG
TGGCTTCAGGCCGCCATCCACTGCGGAGCCGTACAAATGTACGGCCAGCAACGTCGGTT
CGAGATGGCGCTCGATGACGCCAACTACCTCTGATAGTTGAGTCGATACTTCGGCGATC
ACCGCTTCCCTCATACTCTTCCTTTTTCAATATTATTGAAGCATTTATCAGGGTTATTG
TCTCATGAGCGGATACATATTTGAATGTATTTAGAAAAATAAACAAATAGCCAGCTCAC
TCGGTCGCTACGCTCCGGGCGTGAGACTGCGGCGGGCGCTGCGGACACATACAAAGTTA
CCCACAGATTCCGTGGATAAGCAGGGGACTAACATGTGAGGCAAAACAGCAGGGCCGCG
CCGGTGGCGTTTTTCCATAGGCTCCGCCCTCCTGCCAGAGTTCACATAAACAGACGCTT
TTCCGGTGCATCTGTGGGAGCCGTGAGGCTCAACCATGAATCTGACAGTACGGGCGAAA
CCCGACAGGACTTAAAGATCCCCACCGTTTCCGGCGGGTCGCTCCCTCTTGCGCTCTCC
TGTTCCGACCCTGCCGTTTACCGGATACCTGTTCCGCCTTTCTCCCTTACGGGAAGTGT
GGCGCTTTCTCATAGCTCACACACTGGTATCTCGGCTCGGTGTAGGTCGTTCGCTCCAA
GCTGGGCTGTAAGCAAGAACTCCCCGTTCAGCCCGACTGCTGCGCCTTATCCGGTAACT
GTTCACTTGAGTCCAACCCGGAAAAGCACGGTAAAACGCCACTGGCAGCAGCCATTGGT
AACTGGGAGTTCGCAGAGGATTTGTTTAGCTAAACACGCGGTTGCTCTTGAAGTGTGCG
CCAAAGTCCGGCTACACTGGAAGGACAGATTTGGTTGCTGTGCTCTGCGAAAGCCAGTT
ACCACGGTTAAGCAGTTCCCCAACTGACTTAACCTTCGATCAAACCACCTCCCCAGGTG
GTTTTTTCGTTTACAGGGCAAAAGATTACGCGCAGAAAAAAAGGATCTCAAGAAGATCC
TTTGATCTTTTCTACTGAACCGCTCTAGATTTCAGTGCAATTTATCTCTTCAAATGTAG
CACCTGAAGTCAGCCCCATACGATATAAGTTGTAATTCTCATGTTAGTCATGCCCCGCG
CCCACCGGAAGGAGCTGACTGGGTTGAAGGCTCTCAAGGGCATCGGTCGAGATCCCGGT
GCCTAATGAGTGAGCTAACTTACATTAATTGCGTTGCGCTCACTGCCCGCTTTCCAGTC
GGGAAACCTGTCGTGCCAGCTGCATTAATGAATCGGCCAACGCGCGGGGAGAGGCGGTT
TGCGTATTGGGCGCCAGGGTGGTTTTTCTTTTCACCAGTGAGACGGGCAACAGCTGATT
GCCCTTCACCGCCTGGCCCTGAGAGAGTTGCAGCAAGCGGTCCACGCTGGTTTGCCCCA
GCAGGCGAAAATCCTGTTTGATGGTGGTTAACGGCGGGATATAACATGAGCTGTCTTCG
GTATCGTCGTATCCCACTACCGAGATGTCCGCACCAACGCGCAGCCCGGACTCGGTAAT
GGCGCGCATTGCGCCCAGCGCCATCTGATCGTTGGCAACCAGCATCGCAGTGGGAACGA
TGCCCTCATTCAGCATTTGCATGGTTTGTTGAAAACCGGACATGGCACTCCAGTCGCCT
TCCCGTTCCGCTATCGGCTGAATTTGATTGCGAGTGAGATATTTATGCCAGCCAGCCAG
ACGCAGACGCGCCGAGACAGAACTTAATGGGCCC
SEQ.ID NO: 2
GACTACAAGGATGACGATGACAAA
SEQ.ID NO: 3
DYKDDDDK
SEQ.ID NO: 4
ATGAACACCTTCAGAACAGCCACTGCCAGAGACATACCTGATGTAGCAGCAACTCTTAC
GGAAGCCTTCGCAACTGATCCACCCACGCAGTGGGTGTTCCCCGACGGTACTGCCGCCG
TCAGCAGGTTCTTTACACATGTTGCAGATAGGGTTCACACGGCCGGTGGTATTGTTGAG
CTACTACCAGACAGAGCCGCCATGATTGCATTGCCACCACACGTGAGGCTGCCAGGAGA
AGCTGCCGACGGAAGGCAGGCGGAAATTCAGAGAAGGCTGGCAGACAGGCACCCGCTGA
CACCTCACTACTACCTGCTGTTTTACGGAGTTAGAACGGCACACCAGGGTTCGGGATTG
GGCGGAAGAATGCTGGCCAGATTAACTAGCAGAGCTGATAGGGACAGGGTGGGTACATA
TACTGAGGCATCCACCTGGCGTGGCGCTAGACTGATGCTGAGACATGGATTCCATGCTA
CAAGGCCACTAAGATTGCCAGATGGACCCAGCATGTTTCCACTTTGGAGAGATCCAATC
CATGATCATTCTGATTAG
SEQ.ID NO: 5
MNTFRTATARDIPDVAATLTEAFATDPPTQWVFPDGTAAVSRFFTHVADRVHTAGGIVE
LLPDRAAMIALPPHVRLPGEAADGRQAEIQRRLADRHPLTPHYYLLFYGVRTAHQGSGL
GGRMLARLTSRADRDRVGTYTEASTWRGARLMLRHGFHATRPLRLPDGPSMFPLWRDPI
HDHSD
SEQ.ID NO: 6
ATGAAAGGATATTTCGGACCATACGGTGGCCAGTACGTACCAGAAATATTAATGGGTGC
CTTAGAGGAGTTAGAGGCAGCATACGAGGAGATTATGAAGGATGAGAGCTTCTGGAAGG
AGTTCAACGATCTACTGAGGGATTACGCAGGCAGACCAACGCCATTGTACTTTGCCAGG
AGATTGTCTGAGAAGTACGGCGCCCGTGTTTACTTGAAGCGTGAGGATCTGCTGCACAC
TGGAGCACACAAGATAAATAACGCTATCGGACAGGTTTTATTGGCCAAATTAATGGGGA
AGACACGTATCATAGCCGAGACGGGAGCTGGGCAGCATGGAGTCGCTACTGCTACCGCT
GCTGCCCTGTTCGGAATGGAATGTGTGATCTACATGGGTGAAGAGGACACAATCAGACA
GAAGTTGAACGTGGAGCGTATGAAATTATTAGGGGCTAAAGTTGTCCCTGTTAAGTCTG
GCAGTAGGACCTTGAAGGATGCGATAGACGAGGCTTTGAGAGACTGGATTACTAATTTA
CAGACAACATATTATGTTATCGGATCTGTTGTTGGTCCCCACCCTTACCCAATTATCGT
AAGGAATTTCCAGAAGGTTATCGGTGAGGAGACCAAGAAGCAAATACCAGAAAAGGAAG
GTCGTTTGCCAGACTATATAGTTGCCTGCGTAGGCGGCGGTAGCAATGCCGCAGGTATA
TTTTACCCATTCATAGACTCTGGAGTAAAGCTGATAGGTGTTGAGGCAGGTGGCGAGGG
ATTGGAGACAGGTAAACACGCAGCCTCGTTATTAAAGGGTAAAATTGGCTATTTACATG
GATCGAAGACCTTTGTTCTACAAGATGACTGGGGTCAAGTCCAAGTGAGCCATTCGGTG
TCAGCTGGTCTTGACTATTCAGGAGTAGGACCTGAGCATGCTTATTGGAGAGAGACAGG
GAAGGTTCTGTACGACGCAGTGACTGACGAAGAGGCTTTGGACGCATTTATAGAGTTAT
CAAGACTAGAGGGCATTATACCCGCTTTAGAGTCATCGCATGCTCTAGCATATTTGAAG
AAGATAAATATAAAAGGTAAGGTTGTGGTGGTCAACCTATCAGGGAGAGGGGATAAAGA
CCTGGAGTCAGTCTTAAACCATCCATACGTGAGAGAAAGAATTAGATGA
SEQ.ID NO: 7
MKGYFGPYGGQYVPEILMGALEELEAAYEEIMKDESFWKEFNDLLRDYAGRPTPLYFAR
RLSEKYGARVYLKREDLLHTGAHKINNAIGQVLLAKLMGKTRIIAETGAGQHGVATATA
AALFGMECVIYMGEEDTIRQKLNVERMKLLGAKVVPVKSGSRTLKDAIDEALRDWITNL
QTTYYVIGSVVGPHPYPIIVRNFQKVIGEETKKQIPEKEGRLPDYIVACVGGGSNAAGI
FYPFIDSGVKLIGVEAGGEGLETGKHAASLLKGKIGYLHGSKTFVLQDDWGQVQVSHSV
SAGLDYSGVGPEHAYWRETGKVLYDAVTDEEALDAFIELSRLEGIIPALESSHALAYLK
KINIKGKVVVVNLSGRGDKDLESVLNHPYVRERIR
SEQ.ID NO: 8
GGTAAGCCAATTCCAAATCCTTTGTTGGGTTTGGACTCCACC
SEQ.ID NO: 9
GKPIPNPLLGLDST
SEQ.ID NO: 10
GAAGAATTGTGAGCGGATAACAATTCCCCTCTAGAAATAATTTTGTTTAACTTTAAGAA
GGAGATATACATATGGCAGATCTCAATTGGATATCGGCCGGCCACGCGATCGCTGACGT
CGGTACCCTCGAGTCTGGTAAAGAAACCGCTGCTGCGAAATTTGAACGCCAGCACATGG
ACTCGTCTACTAGTCGCAGCTTAATTAACCTAAACTGCTGCCACCGCTGAGCAATAACT
AGCATAACCCCTTGGGGCCTCTAAACGGGTCTTGAGGGGTTTTTTGCTAGCGAAAGGAG
GAGTCGACTATATCCGGATTGGCGAATGGGACGCGCCCTGTAGCGGCGCATTAAGCGCG
GCGGGTGTGGTGGTTACGCGCAGCGTGACCGCTACACTTGCCAGCGCCCTAGCGCCCGC
TCCTTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACCCCAAA
AAACTTGATTAGGGTGATGGTTCACGTAGTGGGCCATCGCCCTGATAGACGGTTTTTCG
CCCTTTGACGTTGGAGTCCACGTTCTTTAATAGTGGACTCTTGTTCCAAACTGGAACAA
CACTCAACCCTATCTCGGTCTATTCTTTTGATTTATAAGGGATTTTGCCGATTTCGGCC
TATTGGTTAAAAAATGAGCTGATTTAACAAAAATTTAACGCGAATTTTAACAAAATATT
AACGTTTACAATTTCTGGCGGCACGATGGCATGAGATTATCAAAAAGGATCTTCACCTA
GATCCTTTTAAATTAAAAATGAAGTTTTAAATCAATCTAAAGTATATATGAGTAAACTT
GGTCTGACAGTTACCAATGCTTAATCAGTGAGGCACCTATCTCAGCGATCTGTCTATTT
CGTTCATCCATAGTTGCCTGACTCCCCGTCGTGTAGATAACTACGATACGGGAGGGCTT
ACCATCTGGCCCCAGTGCTGCAATGATACCGCGAGACCCACGCTCACCGGCTCCAGATT
TATCAGCAATAAACCAGCCAGCCGGAAGGGCCGAGCGCAGAAGTGGTCCTGCAACTTTA
TCCGCCTCCATCCAGTCTATTAATTGTTGCCGGGAAGCTAGAGTAAGTAGTTCGCCAGT
TAATAGTTTGCGCAACGTTGTTGCCATTGCTACAGGCATCGTGGTGTCACGCTCGTCGT
TTGGTATGGCTTCATTCAGCTCCGGTTCCCAACGATCAAGGCGAGTTACATGATCCCCC
ATGTTGTGCAAAAAAGCGGTTAGCTCCTTCGGTCCTCCGATCGTTGTCAGAAGTAAGTT
GGCCGCAGTGTTATCACTCATGGTTATGGCAGCACTGCATAATTCTCTTACTGTCATGC
CATCCGTAAGATGCTTTTCTGTGACTGGTGAGTACTCAACCAAGTCATTCTGAGAATAG
TGTATGCGGCGACCGAGTTGCTCTTGCCCGGCGTCAATACGGGATAATACCGCGCCACA
TAGCAGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTGATCT
TCAGCATCTTTTACTTTCACCAGCGTTTCTGGGTGAGCAAAAACAGGAAGGCAAAATGC
CGCAAAAAAGGGAATAAGGGCGACACGGAAATGTTGAATACTCATACTCTTCCTTTTTC
AATCATGATTGAAGCATTTATCAGGGTTATTGTCTCATGAGCGGATACATATTTGAATG
TATTTAGAAAAATAAACAAATAGGTCATGACCAAAATCCCTTAACGTGAGTTTTCGTTC
CACTGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGAGCGCAGATA
CCAAATACTGTCCTTCTAGTGTAGCCGTAGTTAGGCCACCACTTCAAGAACTCTGTAGC
ACCGCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGG
ACAGGTATCCGGTAAGCGGCAGGGTCGGAACAGGAGAGCGCACGAGGGAGCTTCCAGGG
GGAAACGCCTGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCAACGCGGCCT
TTTTACGGTTCCTGGCCTTTTGCTGGCCTTTTGCTCACATGTTCTTTCCTGCGTTATCC
CCTGATTCTGTGGATAACCGTATTACCGCCTTTGAGTGAGCTGATACCGCTCGCCGCAG
CCGAACGACCGAGCGCAGCGAGTCAGTGAGCGAGGAAGCGGAAGAGCGCCTGATGCGGT
ATTTTCTCCTTACGCATCTGTGCGGTATTTCACACCGCATATATGGTGCACTCTCAGTA
CAATCTGCTCTGATGCCGCATAGTTAAGCCAGTATACACTCCGCTATCGCTACGTGACT
GGGTCATGGCTGCGCCCCGACACCCGCCAACACCCGCTGACGCGCCCTGACGGGCTTGT
CTGCTCCCGGCATCCGCTTACAGACAAGCTGTGACCGTCTCCGGGAGCTGCATGTGTCA
GAGGTTTTCACCGTCATCACCGAAACGCGCGAGGCAGCTGCGGTAAAGCTCATCAGCGT
GGTCGTGAAGCGATTCACAGATGTCTGCCTGTTCATCCGCGTCCAGCTCGTTGAGTTTC
TCCAGAAGCGTTAATGTCTGGCTTCTGATAAAGCGGGCCATGTTAAGGGCGGTTTTTTC
CTGTTTGGTCACTGATGCCTCCGTGTAAGGGGGATTTCTGTTCATGGGGGTAATGATAC
CGATGAAACGAGAGAGGATGCTCACGATACGGGTTACTGATGATGAACATGCCCGGTTA
CTGGAACGTTGTGAGGGTAAACAACTGGCGGTATGGATGCGGCGGGACCAGAGAAAAAT
CACTCAGGGTCAATGCCAGCGCTTCGTTAATACAGATGTAGGTGTTCCACAGGGTAGCC
AGCAGCATCCTGCGATGCAGATCCGGAACATAATGGTGCAGGGCGCTGACTTCCGCGTT
TCCAGACTTTACGAAACACGGAAACCGAAGACCATTCATGTTGTTGCTCAGGTCGCAGA
CGTTTTGCAGCAGCAGTCGCTTCACGTTCGCTCGCGTATCGGTGATTCATTCTGCTAAC
CAGTAAGGCAACCCCGCCAGCCTAGCCGGGTCCTCAACGACAGGAGCACGATCATGCTA
GTCATGCCCCGCGCCCACCGGAAGGAGCTGACTGGGTTGAAGGCTCTCAAGGGCATCGG
TCGAGATCCCGGTGCCTAATGAGTGAGCTAACTTACATTAATTGCGTTGCGCTCACTGC
CCGCTTTCCAGTCGGGAAACCTGTCGTGCCAGCTGCATTAATGAATCGGCCAACGCGCG
GGGAGAGGCGGTTTGCGTATTGGGCGCCAGGGTGGTTTTTCTTTTCACCAGTGAGACGG
GCAACAGCTGATTGCCCTTCACCGCCTGGCCCTGAGAGAGTTGCAGCAAGCGGTCCACG
CTGGTTTGCCCCAGCAGGCGAAAATCCTGTTTGATGGTGGTTAACGGCGGGATATAACA
TGAGCTGTCTTCGGTATCGTCGTATCCCACTACCGAGATGTCCGCACCAACGCGCAGCC
CGGACTCGGTAATGGCGCGCATTGCGCCCAGCGCCATCTGATCGTTGGCAACCAGCATC
GCAGTGGGAACGATGCCCTCATTCAGCATTTGCATGGTTTGTTGAAAACCGGACATGGC
ACTCCAGTCGCCTTCCCGTTCCGCTATCGGCTGAATTTGATTGCGAGTGAGATATTTAT
GCCAGCCAGCCAGACGCAGACGCGCCGAGACAGAACTTAATGGGCCCGCTAACAGCGCG
ATTTGCTGGTGACCCAATGCGACCAGATGCTCCACGCCCAGTCGCGTACCGTCTTCATG
GGAGAAAATAATACTGTTGATGGGTGTCTGGTCAGAGACATCAAGAAATAACGCCGGAA
CATTAGTGCAGGCAGCTTCCACAGCAATGGCATCCTGGTCATCCAGCGGATAGTTAATG
ATCAGCCCACTGACGCGTTGCGCGAGAAGATTGTGCACCGCCGCTTTACAGGCTTCGAC
GCCGCTTCGTTCTACCATCGACACCACCACGCTGGCACCCAGTTGATCGGCGCGAGATT
TAATCGCCGCGACAATTTGCGACGGCGCGTGCAGGGCCAGACTGGAGGTGGCAACGCCA
ATCAGCAACGACTGTTTGCCCGCCAGTTGTTGTGCCACGCGGTTGGGAATGTAATTCAG
CTCCGCCATCGCCGCTTCCACTTTTTCCCGCGTTTTCGCAGAAACGTGGCTGGCCTGGT
TCACCACGCGGGAAACGGTCTGATAAGAGACACCGGCATACTCTGCGACATCGTATAAC
GTTACTGGTTTCACATTCACCACCCTGAATTGACTCTCTTCCGGGCGCTATCATGCCAT
ACCGCGAAAGGTTTTGCGCCATTCGATGGTGTCCGGGATCTCGACGCTCTCCCTTATGC
GACTCCTGCATTAGGAAGCAGCCCAGTAGTAGGTTGAGGCCGTTGAGCACCGCCGCCGC
AAGGAATGGTGCATGCAAGGAGATGGCGCCCAACAGTCCCCCGGCCACGGGGCCTGCCA
CCATACCCACGCCGAAACAAGCGCTCATGAGCCCGAAGTGGCGAGCCCGATCTTCCCCA
TCGGTGATGTCGGCGATATAGGCGCCAGCAACCGCACCTGTGGCGCCGGTGATGCCGGC
CACGATGCGTCCGGCGTAGCCTAGGATCGAGATCGATCTCGATCCCGCGAAATTAATAC
GACTCACTACG
SEQ.ID NO: 11
ATGATGTCTGAAAATTTGCAATTGTCAGCTGAAGAAATGAGACAATTGGGTTACCAAGC
AGTTGATTTGATCATCGATCACATGAACCATTTGAAGTCTAAGCCAGTTTCAGAAACAA
TCGATTCTGATATCTTGAGAAATAAGTTGACTGAATCTATCCCAGAAAATGGTTCAGAT
CCAAAGGAATTGTTGCATTTCTTGAACAGAAACGTTTTTAATCAAATTACACATGTTGA
TCATCCACATTTCTTGGCTTTTGTTCCAGGTCCAAATAATTACGTTGGTGTTGTTGCAG
ATTTCTTGGCTTCTGGTTTTAATGTTTTTCCAACTGCATGGATTGCTGGTGCAGGTGCT
GAACAAATCGAATTGACTACAATTAATTGGTTGAAATCTATGTTGGGTTTTCCAGATTC
AGCTGAAGGTTTATTTGTTTCTGGTGGTTCAATGGCAAATTTGACAGCTTTGACTGTTG
CAAGACAGGCTAAGTTGAACAACGATATCGAAAATGCTGTTGTTTACTTCTCTGATCAA
ACACATTTCTCAGTTGATAGAGCATTGAAGGTTTTAGGTTTTAAACATCATCAAATCTG
TAGAATCGAAACAGATGAACATTTGAGAATCTCTGTTTCAGCTTTGAAGAAACAAATTA
AAGAAGATAGAACTAAGGGTAAAAAGCCATTCTGTGTTATTGCAAATGCTGGTACTACA
AATTGTGGTGCTGTTGATTCTTTGAACGAATTAGCAGATTTGTGTAACGATGAAGATGT
TTGGTTGCATGCTGATGGTTCTTATGGTGCTCCAGCTATCTTGTCTGAAAAGGGTTCAG
CTATGTTGCAAGGTATTCATAGAGCAGATTCTTTGACTTTAGATCCACATAAGTGGTTG
TTCCAACCATACGATGTTGGTTGTGTTTTGATCAGAAACTCTCAATATTTGTCAAAGAC
TTTTAGAATGATGCCAGAATACATCAAGGATTCAGAAACTAACGTTGAAGGTGAAATTA
ATTTCGGTGAATGTGGTATCGAATTGTCAAGAAGATTCAGAGCTTTGAAGGTTTGGTTG
TCTTTTAAAGTTTTCGGTGTTGCTGCTTTTAGACAAGCAATCGATCATGGTATCATGTT
AGCAGAACAAGTTGAAGCATTTTTGGGTAAAGCAAAAGATTGGGAAGTTGTTACACCAG
CTCAATTGGGTATCGTTACTTTTAGATACATTCCATCTGAATTGGCATCAACAGATACT
ATTAATGAAATTAATAAGAAATTGGTTAAGGAAATCACACATAGAGGTTTCGCTATGTT
ATCTACTACAGAATTGAAGGAAAAGGTTGTTATTAGATTGTGTTCAATTAATCCAAGAA
CTACAACTGAAGAAATGTTGCAAATCATGATGAAGATTAAAGCATTGGCTGAAGAAGTT
TCTATTTCATACCCATGTGTTGCTGAATAA
SEQ.ID NO: 12
MMSENLQLSAEEMRQLGYQAVDLIIDHMNHLKSKPVSETIDSDILRNKLTESIPENGSD
PKELLHFLNRNVFNQITHVDHPHFLAFVPGPNNYVGVVADFLASGFNVFPTAWIAGAGA
EQIELTTINWLKSMLGFPDSAEGLFVSGGSMANLTALTVARQAKLNNDIENAVVYFSDQ
THFSVDRALKVLGFKHHQICRIETDEHLRISVSALKKQIKEDRTKGKKPFCVIANAGTT
NCGAVDSLNELADLCNDEDVWLHADGSYGAPAILSEKGSAMLQGIHRADSLTLDPHKWL
FQPYDVGCVLIRNSQYLSKTFRMMPEYIKDSETNVEGEINFGECGIELSRRFRALKVWL
SFKVFGVAAFRQAIDHGIMLAEQVEAFLGKAKDWEVVTPAQLGIVTFRYIPSELASTDT
INEINKKLVKEITHRGFAMLSTTELKEKVVIRLCSINPRTTTEEMLQIMMKIKALAEEV
SISYPCVAE

### EXAMPLES

### Example 1—Processes for Making a 4-benzyloxy-7-nitro Psilocybin Derivative

Trifluoroacetic acid (1.34 mL) was added to a mixture of 4-benzoxylindole (9A-1, 300 mg, 1.34 mmol) and dimethylamino-2-nitroethylene (170 mg, 1.46 mmol). The reaction mixture was stirred at room temperature for forty minutes before poured it into a mixture of EtOAc (7.8 mL) and 10% aqueous Na₂CO₃ (23.5 mL). The layers were separated, and the aqueous phase was extracted with EtOAc (4×16 mL). The combined organic solutions were washed with brine (40 mL) and dried over anhydrous MgSO₄. The organic solvent was concentrated in vacuo. The product was purified by flash chromatography on silica gel (eluted with a gradient of hexanes-DCM, 100:00 to 00:100) to afford compound 9A-2 as a red solid. Yield: 12%. ¹H NMR (400 MHz, CDCl₃) δ 8.59 (d, J=13.8 Hz, 1H), 7.83 (d, J=13.5 Hz, 1H), 7.59 (d, J=3.0 Hz, 1H), 7.55-7.51 (m, 2H), 7.48-7.42 (m, 2H), 7.41-7.35 (m, 1H), 7.22 (t, J=8.0 Hz, 1H), 7.06 (dd, J=8.2, 0.7 Hz, 1H), 6.76 (d, J=7.9 Hz, 1H), 5.28 (s, 2H).

1.0 M of lithium aluminum in THF (16.3 mL, 16.3 mmol) was added dropwise to a stirred solution of compound 9A-2 (215 mg, 0.73 mmol) in anhydrous THF (19.3 mL) in an ice-water bath. The reaction mixture was stirred at room temperature for three days then heated to reflux for 16 hours. Once the reaction mixture was cooled to room temperature, 10% water/THF mixture was added until hydrogen gas evaluation ceased. The precipitate was filtered, and the filtrate was dried over anhydrous MgSO₄. The organic solvent was concentrated under reduced pressure to afford a brown oil (9A-4), which was used in the next step without further purification.

To a solution of compound 9A-4 (60.4 mg, 0.23 mmol) in anhydrous acetonitrile (1.5 mL) was added Di-tert-butyl decarbonate (350 mg, 1.60 mmol) and DMAP (14 mg, 0.11 mmol). The reaction mixture was stirred at room temperature for 16 hours. Water (10 mL) was added to the crude product, extracted in dichloromethane (3×10 mL). The combined organic solution was washed with brine (15 mL), dried over anhydrous MgSO₄, and concentrated under reduced pressure. The crude mixture was purified by flash column chromatography on silica gel (eluted with a gradient of DCM-MeOH, 100:0 to 00:03) to afford compound 9A-4 as a yellow oil. Yield: 43%. ¹H NMR (400 MHz, CDCl₃) δ 7.78 (d, J=8.4 Hz, 1H), 7.48-7.42 (m, 2H), 7.42-7.29 (m, 4H), 7.18 (t, J=8.2 Hz, 1H), 6.71 (d, J=7.9 Hz, 1H), 5.20 (d, J=1.7 Hz, 2H), 1.64 (s, 9H), 1.42 (s, 9H). HRMS (ESI, positive) m/z for C₂₇H₃₅N₂O₅ [M+H]⁺ calcd. 467.2541, found 467.2535.

Compound 9B-5 (46 mg, 0.10 mmol) and silver nitrate (19 mg, 0.11 mmol) were dissolved in anhydrous acetonitrile (0.3 mL). The reaction mixture was cooled in an ice-water bath. Benzoyl chloride (13 μL, 0.11 mmol) was added dropwise to the cooled solution and the reaction mixture was stirred at the same temperature. Once TLC showed the consumption of the starting material, water (6.5 mL) was added, and the reaction mixture was extracted with EtOAc (3×10 mL). The combined organic solutions were washed with saturated sodium carbonate (20 mL), dried over anhydrous MgSO₄, and concentrated under reduced pressure. The crude material was purified by flash column chromatography on silica gel (eluted with a gradient of hexanes-EtOAc, 100:0 to 00:100) to afford compound 9B-6 which is contaminated with other nitrated compounds as an inseparable mixture.

Trifluoroacetic acid (15 μL, 0.20 mmol) was added to a solution of the above mixture (14.2 mg, 0.02 mmol) in dichloromethane (0.2 mL). The reaction mixture was stirred at room temperature for two hours and thirty minutes then neutralized with saturated sodium carbonate solution. The mixture was extracted with dichloromethane (3×10 mL), dried over anhydrous MgSO₄, and concentrated under reduced pressure. The crude material was purified by flash column chromatography on silica gel (eluted with a gradient of DCM-MeOH, 100:00 to 80:20) to afford the product 9B-7 as an orange oil. ¹H NMR (400 MHz, CDCl₃) δ 9.85 (s, 1H), 8.19 (d, J=9.0 Hz, 1H), 7.50-7.41 (m, 6H), 7.08 (d, J=2.2 Hz, 1H), 6.69 (d, J=8.9 Hz, 1H), 5.29 (s, 2H), 3.51 (q, J=6.7 Hz, 2H), 3.05 (t, J=7.0 Hz, 2H), 2.04 (bs, 6H). HRMS (ESI, positive) m/z for C₁₇H₁₈N₃O₃ [M+H]⁺ calcd. 312.1343, found 312.1344.

### Example 2—Processes for Making 4-methoxy-7-nitro-Psilocybin, 4-methoxy-5-nitro-Psilocybin and 4-methoxy-2-nitro-Psilocybin Derivatives

Trifluoroacetic acid (8.2 mL) was added to a mixture of 4-methoxyindole (9B-1, 1.20 g, 8.15 mmol) and dimethylamino-2-nitroethylene (1.04 g, 8.96 mmol). The reaction mixture was stirred at room temperature for an hour before poured it into a mixture of EtOAc (52 mL) and 10% aqueous Na₂CO₃ (72 mL). The layers were separated, and the aqueous phase was extracted with EtOAc (3×100 mL). The combined organic solutions were washed with brine and dried over anhydrous MgSO₄. The organic solvent was concentrated in vacuo. The crude product (E)-4-methoxy-3-(2-nitrovinyl)indole (9B-2) was used directly without further purification.

To the crude (E)-4-methoxy-3-(2-nitrovinyl)indole (9B-2) in EtOH (40.0 mL) and THF (40.0 mL) was added sodium borohydride (1.24 g, 32.76 mmol). The reaction mixture was stirred at room temperature for an hour and 30 minutes. The reaction mixture was carefully quenched with ice-water (852 mL) and extracted with dichloromethane (3×400 mL). The combined organic solutions were washed with brine and dried over anhydrous MgSO₄. The organic solvent was concentrated in vacuo. The product was purified by flash chromatography on silica gel (eluted with a gradient of hexanes-dichloromethane, 20:80-0:100) to afford the desired 4-methoxy-3-(2-nitroethyl)indole (9B-3) as a yellow solid. Yield: 11% (over two steps). The ¹H NMR spectrum agreed with previously reported procedure (Vo, Q. V; Trenerry, C.; Rochfort, S.; Wadeson, J.; Leyton, C.; Hughes, A. Bioorg. Med. Chem. 2014, 22, 856-864). ¹H NMR (400 MHz, CDCl₃) δ 7.99 (s, 1H), 7.12 (t, J=8.0 Hz, 1H), 6.97 (dd, J=8.2, 0.7 Hz, 1H), 6.92 (d, J=2.3 Hz, 1H), 6.52 (d, J=7.8 Hz, 1H), 4.73 (t, J=7.2 Hz, 2H), 3.94 (s, 3H), 3.54 (t, J=7.2 Hz, 2H).

A solution of 1.0 M of lithium aluminum in THF (4.6 mL, 4.6 mmol) was added to a cooled solution of 4-methoxy-3-(2-nitroethyl)indole (9B-3, 202 mg, 0.92 mmol) in anhydrous THF (9.2 mL). The reaction mixture was warmed to room temperature then heated to reflux. After three hours, the reaction mixture was cooled in an ice-water bath and was quenched with 10% water/THF until no more hydrogen gas evaluation. The precipitate was filtered, and the filtrate was dried over anhydrous MgSO₄. The organic solvent was concentrated under reduced pressure to afford the desired 4-methoxy psilocybin derivative (9B-4) as an orange solid, which was used in the next step without further purification. Yield: 73%. The ¹H NMR spectrum agreed with previously reported procedure (Kerschgens, I.; Claveau, E.; Wanner, M.; Ingemann, S.; Maarseveen, J. H.; Hiemstra, H. Chem. Commun. 2012, 48, 12243-12245). ¹H NMR (400 MHz, CDCl₃) δ 8.13 (bs, 1H), 7.08 (t, J=7.9 Hz, 1H), 6.96 (d, J=8.1 Hz, 1H), 6.90-6.85 (m, 1H), 6.49 (d, J=7.8 Hz, 1H), 3.91 (s, 3H), 3.01 (m, 4H).

To a solution of crude 4-methoxy psilocybin derivative (9B-4, 128 mg, 0.67 mmol) in anhydrous acetonitrile (4.3 mL) was added Di-tert-butyl dicarbonate (730 mg, 3.34 mmol) and DMAP (41.0 mg, 0.33 mmol). The reaction mixture was stirred at room temperature overnight. The reaction mixture was diluted with water (20 mL) and extracted in dichloromethane (3×20 mL). The combined organic solution was washed with brine (30 mL), dried over anhydrous MgSO₄, and concentrated under reduced pressure. The crude mixture was purified by flash column chromatography on silica gel (eluted with a gradient of hexanes-dichloromethane, 100:0->0:100) to afford the N1,N,N-triBoc protected 4-methoxy psilocybin derivative 9B-5 as a yellow oil. Yield: 45%. ¹H NMR (400 MHz, CDCl₃) δ 7.74 (d, J=8.4 Hz, 1H), 7.22-7.16 (m, 2H), 6.64 (d, J=7.9 Hz, 1H), 3.96-3.89 (m, 5H), 3.08 (t, J=6.7 Hz, 2H), 1.63 (s, 9H), 1.39 (s, 18H). 13C NMR (100 MHz, CDCl₃) δ 154.4, 152.7, 149.7, 137.2, 125.1, 122.2, 120.0, 118.0, 108.3, 103.2, 83.3, 81.8, 55.2, 46.9, 28.2, 27.9, 26.6. HRMS (ESI, positive) m/z for C₁₆H₂₃N₂O₃ [M−2Boc+H]⁺ calcd. 291.1703, found 291.1705.

The N1,N,N-triBoc protected 4-methoxy psilocybin derivative (9B-5, 325 mg, 0.662 mmol) and silver nitrate (124.0 mg, 0.729 mmol) were dissolved in anhydrous acetonitrile (2.5 mL). The reaction mixture was cooled in an ice-water bath. Benzoyl chloride (102 mg, 0.729 mmol) was added dropwise to the cooled solution and the reaction mixture was stirred at the same temperature for three hours. The reaction mixture was diluted with ethyl acetate (10 mL) and the precipitated salts were removed via vacuum filtration and washed with ethyl acetate (5 mL). The organic filtrate was washed with water (3×20 mL) and saturated Na₂CO₃ (20 mL), then dried with MgSO₄ and solvent removed in vacuo. The crude mixture was purified by column chromatography on silica gel using a gradient of 5 to 15% ethyl acetate—hexanes to afford compounds 9B-7 (48 mg, 0.090 mmol, 14%), 9B-8 (25 mg, 0.047 mmol, 7%), and 9B-6 (45 mg, 0.078 mmol, 12%) in order of elution as yellow solids. ¹H NMR data for 9B-6 (400 MHz, CDCl₃) δ 7.82 (d, J=8.7 Hz, 1H), 7.26 (s, 2H), 6.64 (d, J=8.8 Hz, 1H), 4.02 (s, 3H), 3.91 (t, J=6.8 Hz, 2H), 3.08 (t, J=6.8 Hz, 2H), 1.57 (s, 9H), 1.40 (s, 18H). ¹³C NMR (100 MHz, CDCl₃) δ 158.4, 152.7, 148.7, 133.4, 128.2, 125.9, 123.0, 122.4, 117.2, 102.3, 85.5, 82.1, 56.0, 46.6, 28.0, 27.9, 26.1. HRMS (ESI, positive) m/z for C₁₆H₂₂N₃O₅ [M−2Boc+H]⁺ calcd. 336.1554, found 336.1556. ¹H NMR data for 9B-7 (400 MHz, CDCl₃) δ 8.00-7.94 (m, 1H), 7.87 (d, J=9.0 Hz, 1H), 7.38 (s, 1H), 4.01 (s, 3H), 3.94 (t, J=7.6 Hz, 2H), 3.10 (t, J=7.6 Hz, 2H), 1.65 (s, 9H), 1.41 (s, 18H). ¹H NMR data for 9B-8 (400 MHz, CDCl₃) δ 7.62 (dd, J=8.5, 0.7 Hz, 1H), 7.41 (t, J=8.3 Hz, 1H), 7.41 (s, 1H), 6.70 (dd, J=8.1, 0.7 Hz, 1H), 4.07-4.02 (m, 2H), 3.94 (s, 3H), 3.37-3.31 (m, 2H), 1.55 (s, 9H), 1.33 (s, 18H).

Trifluoroacetic acid (51 μL, 0.67 mmol) was added to a mixture of compound 9B-6 (18.9 mg, 0.04 mmol) in dichloromethane (0.4 mL). The reaction mixture was stirred at room temperature for five hours and 40 minutes then neutralized with saturated sodium bicarbonate solution. The mixture was extracted with dichloromethane (3×10 mL), dried over anhydrous MgSO₄, and concentrated under reduced pressure to afford the 4-methoxy-7-nitro psilocybin derivative trifluoroacetic acid salt (9B-9), as an orange solid. Yield: 29%. ¹H NMR (400 MHz, CDCl₃) δ 9.87 (s, 1H), 8.14 (d, J=9.0 Hz, 1H), 7.04 (s, 1H), 6.54 (d, J=9.0 Hz, 1H), 4.03 (s, 3H), 3.00 (s, 4H), 1.64 (bs, 3H). ¹³C NMR (100 MHz, CDCl₃) δ 161.01, 131.90, 122.87, 122.65, 118.85, 116.08, 100.12, 56.00, 42.96, 30.66. HRMS (ESI, positive) m/z for C₁₈H₂₈BrN₂ [M+H]⁺ calcd. 236.1030, found 236.1026.

To a solution of compound 9B-7 (24 mg, 0.045 mmol, 1.0 eq) in dichloromethane (0.5 mL) was added trifluoroacetic acid (69 μL, 0.90 mmol, 20 eq) dropwise. The reaction mixture was stirred at room temperature for 24 hours until completion as determined by TLC (15% MeOH—dichloromethane). The amber mixture was adjusted to pH 9 with saturated NaHCO₃ (5 mL) and extracted with dichloromethane (4×10 mL). The combined organic extracts were concentrated under vacuo to yield compound 9B-10 as an orange solid (10 mg, 0.043 mmol, 95%). ¹H NMR (600 MHz, Chloroform-d) δ 8.84 (s, 1H), 7.81 (d, J=8.9 Hz, 1H), 7.10-7.04 (m, 2H), 4.02 (s, 3H), 3.07 (t, J=6.2 Hz, 2H), 3.05-3.00 (m, 2H).

To a solution of compound 9B-8 (12 mg, 0.022 mmol, 1.0 eq) in dichloromethane (0.5 mL) was added trifluoroacetic acid (35 μL, 0.45 mmol, 20 eq) dropwise. The reaction mixture was stirred at room temperature for 24 hours until completion as determined by TLC (15% MeOH—dichloromethane). The amber mixture was adjusted to pH 9 with saturated NaHCO₃ (3 mL) and extracted with dichloromethane (4×10 mL). The combined organic extracts were concentrated under vacuo to yield compound 9B-11 as an orange solid (5 mg, 0.021 mmol, 95%). ¹H NMR (600 MHz, Chloroform-d) δ 7.32 (t, J=8.1 Hz, 1H), 6.89 (d, J=8.5 Hz, 1H), 6.51 (d, J=7.9 Hz, 1H), 3.96 (s, 3H), 3.55 (t, J=6.8 Hz, 2H), 3.12 (t, J=6.8 Hz, 2H).

Compound 9B-9 has chemical formula (XXIX):

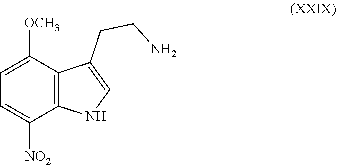 [SMILES: COc1ccc([N+](=O)[O-])c2[nH]cc(CCN)c12]

Yield: 29%. ¹H NMR (400 MHz, CDCl₃) δ 9.87 (s, 1H), 8.14 (d, J=9.0 Hz, 1H), 7.04 (s, 1H), 6.54 (d, J=9.0 Hz, 1H), 4.03 (s, 3H), 3.00 (s, 4H), 1.64 (bs, 3H). ¹³C NMR (100 MHz, CDCl₃) δ 161.01, 131.90, 122.87, 122.65, 118.85, 116.08, 100.12, 56.00, 42.96, 30.66. HRMS (ESI, positive) m/z for C₁₈H₂₈BrN₂ [M+H]⁺ calcd. 236.1030, found 236.1026. The compound having chemical formula (XXIX) was 95% (w/w) pure.

Cell lines for pharmacology assays. CHO-K1/Galpha15 (GenScript, M00257) (−5-HT_{2A}) and CHO-K1/5-HT_{2A} (GenScript, M00250) (+5-HT_{2A}) cells lines were used in both toxicology/growth inhibition (MTT) and calcium release assays. Briefly, CHO-K1/Galpha15 is a control cell line that constitutively expresses Galpha15 which is a promiscuous Gq protein. It is engineered as a host cell, allowing transfected receptor(s) to signal through the Gq signal transduction pathway and mobilize intracellular calcium from the endoplasmic reticulum (ER). These control cells lack any transgene encoding 5-HT_{2A} receptors, thus preventing calcium mobilization in response to 5-HT_{2A} activation. Conversely, CHO-K1/5-HT_{2A} cells stably express 5-HT_{2A} receptor in the CHO-K1 host background. This design enables Gq-11 expressed in CHO-K1 cells to mobilize intracellular calcium changes when 5-HT_{2A} receptors are activated by ligands.

Cell lines were maintained in Ham's F12 media plus 10% FBS in the presence of 100 ug/ml hygromycin for CHO-K1/Ga15 or 400 ug/ml G418 for CHO-K1/5-HT_{2A} unless indicated otherwise for specific assays. Cell maintenance was carried out as recommended by the cell supplier. Briefly, vials with cells were removed from the liquid nitrogen and thawed quickly in 37° C. water bath. Just before cells were completely thawed, vial exteriors were decontaminated with 70% ethanol spray. Cell suspension was then retrieved from the vial and added to warm (37° C.), ‘complete’ (non-dropout) growth media, and centrifuged at 1,000 rpm for 5 minutes. The supernatant was discarded and the cell pellet was then resuspended in another 10 ml of complete growth media, and added to a 10 cm cell culture dish (Greiner Bio-One #664160). The media was changed every third day until the cells reached ˜90% confluence. The ˜90% confluent cells were then split 10:1, and used either for maintenance or pharmacological study.

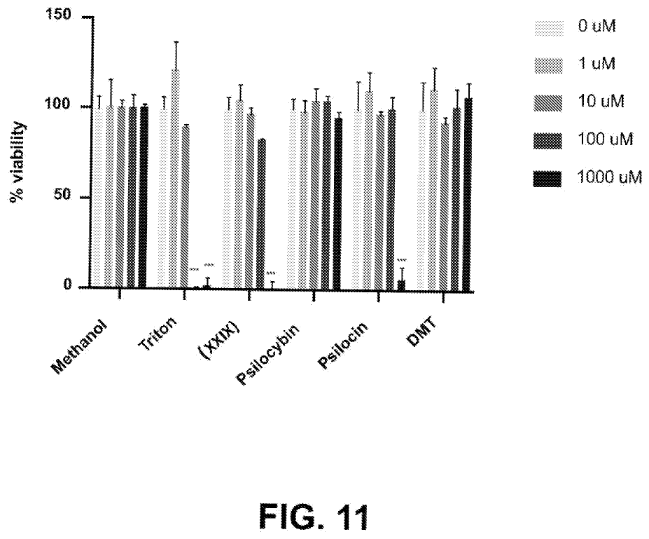

Assessment of cell viability upon treatment with a 4-methoxy-7-nitro psilocybin derivative. To establish suitable ligand concentrations for the calcium release assays, MTT (3-(4,5-dimethylthiazol-2-yl)-2,5-diphenyltetrazolium) assays were first performed. Results of these assays were conducted using both control ligands (e.g. psilocybin, psilocin, DMT) and the novel derivative, in part as a pre-screen for any remarkable toxic effects on cell cultures up to concentrations of 1 mM. A known cellular toxin (Triton X-100, Pyrgiotakis G. et al., 2009, Ann. Biomed. Eng. 37: 1464-1473) was included as a general marker of toxicity. Modified Chinese Hamster Ovary cells (CHO-K1/Ga15) were cultured using standard procedures using the manufacture's protocols (Genscript, M00257). Briefly, cells were cultured in Ham's F12 medium supplemented with 10% fetal bovine serum and 100 mg/ml Hygromycin B, and grown at 37° C. in the presence of 5% CO₂. To test the various compounds with the cell line, cells were seeded in a clear 96-well culture plate at 10,000 cells per well. After allowing cells to attach and grow for 24 hours, assay compounds were added at 1 μM, 10 μM, 100 μM, and 1 mM final concentrations. Methanol concentrations used are 0.001, 0.01, 0.1, and 1%. Triton concentrations used are 0.0001, 0.001, 0.01 and 0.1%. Cells were incubated with compounds for 48 hours before accessing cell viability with the MTT assay following the manufacture's protocol (MTT Cell Growth Assay Kit; Millipore Sigma, CT02). MTT reagent was added to cells and allowed to incubate for 4 hours before solubilization with isopropanol plus 0.04 N HCl. Absorbance readings were performed at 570 nm with the reference at 630 nm on a SpectraMax iD3 plate reader. Non-treated cells were assigned 100% viability. Results of the cell viability assays are shown in FIG. 11. Bar graphs show the mean+/−SD (n=3). Significance (P<0.0001), as indicated by (***) was determined using 2-way ANOVA with Dunnett's multiple comparisons test. The results using compound with formula (XXIX) are indicated as “(XXIX)” on the x-axis.

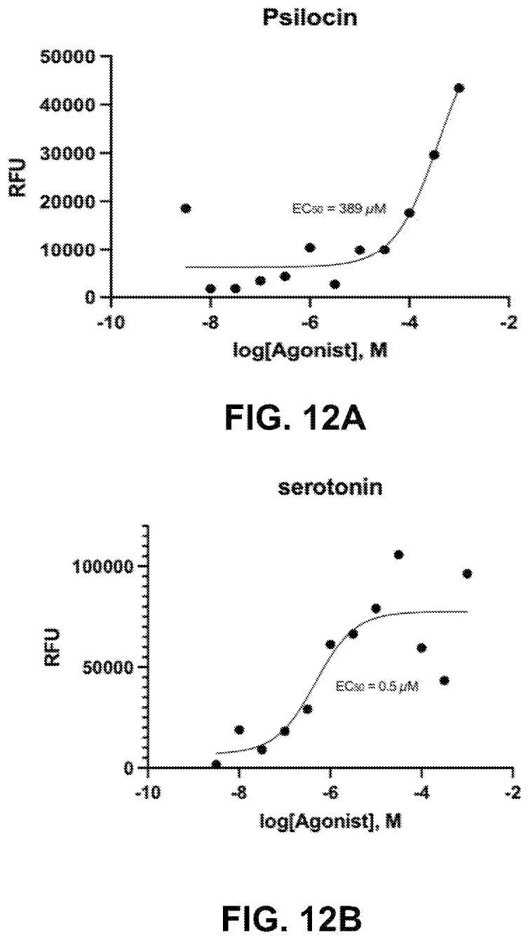

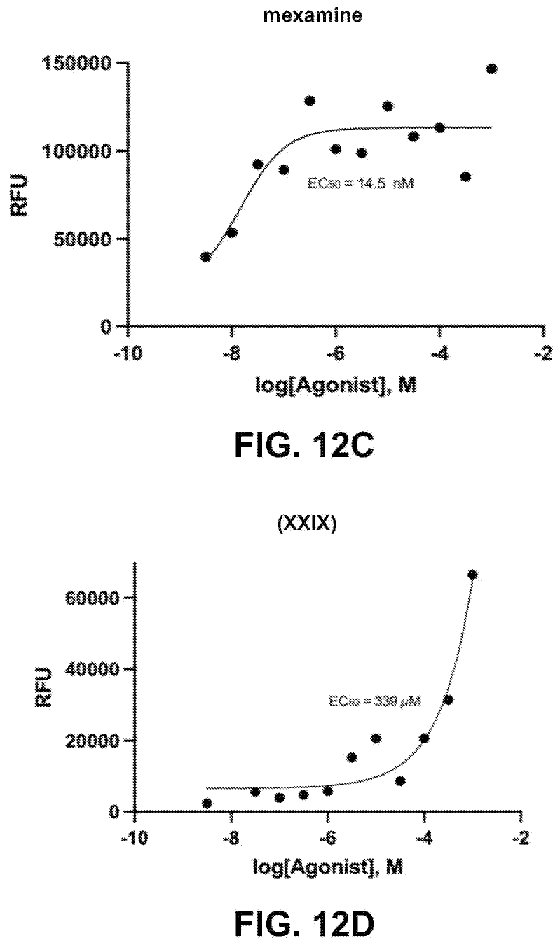

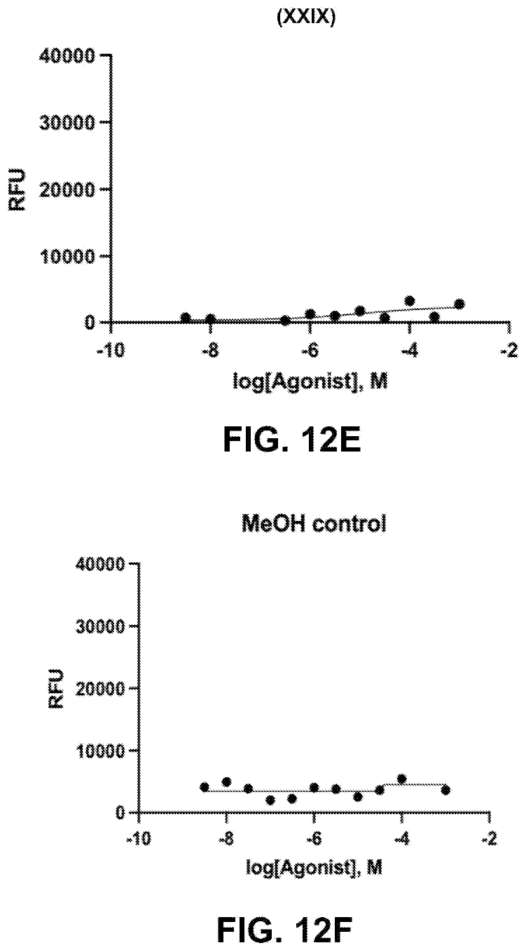

Increase in cytosolic calcium concentration by 5-HT_{2A} activation. Changes in intracellular calcium concentration due to the treatment with assay compounds was measured using Fluo-8 dye (Abcam, #ab112129) according to the manufacturers instructions. Briefly, CHO-K1 cells stably expressing 5-HT_{2A} (Genscript #M00250) (+5-HT_{2A}) or lacking 5-HT_{2A} (Genscript, M00257) (−5-HT_{2A}) were seeded on black walled clear bottom 96-well plates (Thermo Scientific #NUNC165305), allowing 70,000 cells/well in 100 ul media (HAM's F12, GIBCO #11765-047) with 1% FBS (Thermo Scientific #12483020). Cultures were maintained in a humidified incubator at 37° C. and 5% CO₂. Fluo-8 dye was loaded into the cultures for 30 min at 37° C., followed by 30 min additional incubation at room temperature. Next, different dilutions of novel molecules and controls were prepared in serum-free culture media and added to the cells. Fluorescence (ex 490 nm/em 525 nm) obtained after the addition of molecules was expressed relative to values obtained before addition of the molecules (relative Fluo-8 fluorescence=Fmax/F0, where Fmax=maximum fluorescence and F0=baseline fluorescence). Fluorescence intensities were measured using a Spectramax ID3 plate reader (www.moleculardevices.com). Relative fluorescence (RFU) at increasing concentrations of compound was determined, illustrating concentration-dependent calcium flux. Data was subjected to four parameter logistic curve fittings to determine EC₅₀ with the aid of GraphPad Prism (Version 9.2.0). Psilocin (FIG. 12A), serotonin (FIG. 12B) and mexamine (FIG. 12C) are known agonists with binding activity at 5-HT_{2A} (Rickli A. et al., 2016, Europ. Neuropsychopharmacol., 26: 1326-1337; Toro-Sazo M. et al., 2019, PLoS ONE 14: e0209804) and were used as positive controls to establish assay functionality. The Example compound having chemical formula (XXIX) was then evaluated in +5-HT_{2A} (FIG. 12D) and −5-HT_{2A} cell cultures (FIG. 12E). Results using methanol as negative control are shown in FIG. 12F.

### Example 3—Process for Biosynthetically Making a First Nitrated Psilocybin Derivative from Nitrated Indole Feedstock

E. coli strain Ec-1 was constructed as follows. For plasmid cloning, Top10 or XL1-blue strains were used depending on antibiotic markers. Standard LB media was used for culturing. For gene expression and feeding experiments, the parent host strain employed was BL21 (DE3). From plasmid pCDM4 (SEQ.ID NO: 1), the plasmid pCDM4-PsmF-FLAG was created by inserting an in-frame, C-terminally FLAG-tagged (SEQ.ID NO: 2, SEQ.ID NO: 3) PsmF gene (SEQ.ID NO: 4, SEQ.ID NO: 5) into the NdeI/XhoI site of pCDM4. The plasmid pETM6-H10-TmTrpB-2F3-V5-BaTDC-FLAG was created by first cloning the in-frame, C-terminally V5-tagged (SEQ.ID NO: 8, SEQ.ID NO: 9) TmTrpB-2F3 (SEQ.ID NO: 6, SEQ.ID NO: 7) into the NdeI/XhoI site of pETM6-H10 (SEQ.ID NO: 10) to create pETM6-H10-TmTrpB-2F3-V5. This intermediate plasmid was digested with SpeI and SalI, and in-frame, C-terminally FLAG tagged (SEQ.ID NO: 2, SEQ.ID NO: 3) BaTDC (SEQ.ID NO: 11, SEQ.ID NO: 12) was cloned into the site with XbaI and SalI, nullifying the SpeI restriction site. In this setup, the T7 polymerase was able to drive the expression of the polycistronic DNA containing both TmTrpB-2F3 and BaTDC. The two target plasmids pCDM4-PsmF-FLAG and pETM6-H10-TmTrpB-2F3-V5-BaTDC-FLAG were transformed into BL21 (DE3) cells, and antibiotics ampicillin plus streptomycin were used to select for the correct clones containing both plasmids. Scaled-up culturing of engineered E. coli was conducted as follows: seed cultures were inoculated in AMM (Jones et al. 2015, Sci Rep. 5: 11301) medium overnight. The overnight culture was then divided into two flasks containing 500 mL each of AMM medium additionally containing 0.5% (w/v) serine, 1M IPTG, 50 ug/L ampicillin and streptomyces, and 100 mg/L 7-nitroindole (BLDPharm, www.bldpharm.com) for conversion by Ec-1. Cultures were grown for 24 h. Cultures were then centrifuged (10,000g×5 minutes) to remove cellular content, and culture broth containing secreted derivative was stored at −80 C until further processing. Analysis was carried out using high-resolution LC-HESI-LTQ-Orbitrap-XL MS (Thermo Fisher Scientific), employing a modified version of a method described previously (Chang et al., 2015, Plant Physiol. 169: 1127-1140), with the exception that liquid chromatography was carried out using an UltiMate 3000 HPLC (Thermo Fisher Scientific) equipped with a Poroshell 120 SB-C₁₈ column (Agilent Technologies) instead of an Accela HPLC system (Thermo Fisher Scientific) equipped with a Zorbax C₁₈ column (Agilent Technologies). Briefly, 10 microliters of culture media was injected at a flow rate of 0.5 mL/min and a gradient of solvent A (water with 0.1% of formic acid) and solvent B (ACN with 0.1% formic acid) as follows: 100% to 0% (v/v) solvent A over 5 min; isocratic at 0% (v/v) for 1 min; 0% to 100% (v/v) over 0.1 min; and isocratic at 100% (v/v) for 1.9 min. Total run time was 8 minutes. Heated ESI source and interface conditions were operated in positive ion mode as follows: vaporizer temperature, 400° C.; source voltage, 3 kV; sheath gas, 60 au, auxiliary gas, 20 au; capillary temperature, 380° C.; capillary voltage, 6 V; tube lens, 45 V. Instrumentation was performed as a single, HR scan event using Orbitrap detection of m/z in the range of 100-500 m/z. Ion injection time was 300 ms with scan time of 1 s. External and internal calibration procedures ensured <2 ppm error to facilitate elemental formulae predictions. Singly protonated product with exact m/z and expected elemental formula matching the singly protonated form of N-[2-(7-nitro-1H-indol-3-yl)ethyl]acetamide having chemical formula (III):

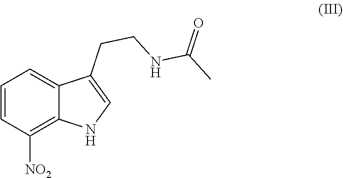 [SMILES: CC(=O)NCCc1c[nH]c2c([N+](=O)[O-])cccc12]

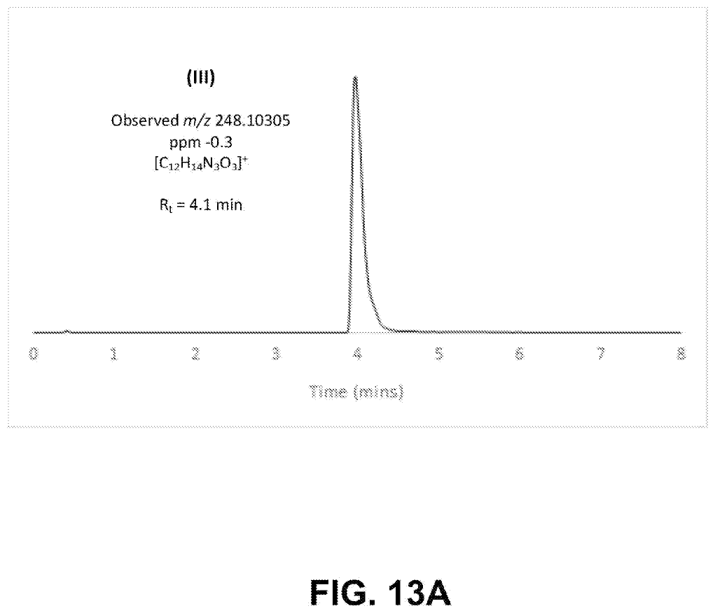

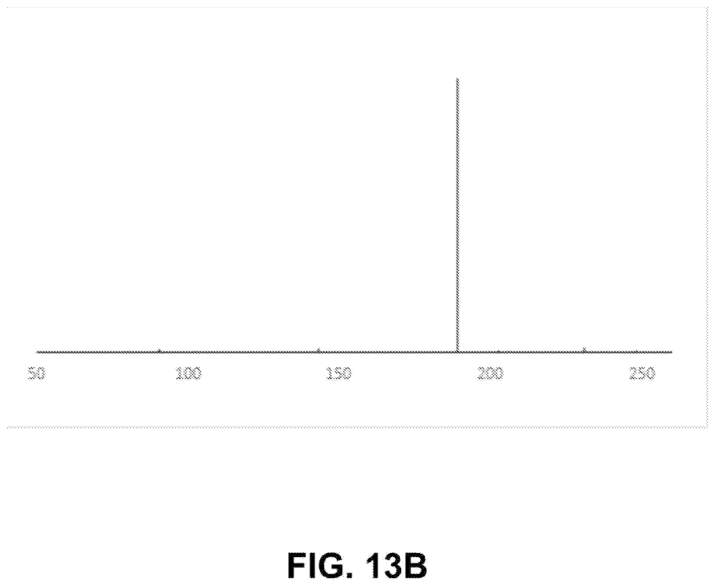

eluted at 4.1 minutes (EIC, see: FIG. 13A).

As per standard procedures (Menéndez-Perdomo et al. 2021, Mass Spectrom 56: 34683) high energy collisions (HCD) were achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of a compound of formula (III), (FIG. 13B, Table 1) (Servillo L. et al., 2013, J. Agric. Chem. 61: 5156-5162).

TABLE I
relative abundance of molecular species in a
sample containing compound (III)

| m/z | % Relative abundance |
|---|---|
| 189.0657 | 100 |
| 231.0764 | 1.6 |
| 143.0728 | 1.3 |
| 90.4071 | 1.0 |
| 202.4614 | 0.6 |
| 248.1030 | 0.5 |
| 80.5873 | 0.3 |
| 102.1377 | 0.2 |

### Example 4—Process for Biosynthetically Making a Second Nitrated Psilocybin Derivative from Nitrated Indole Feedstock

Escherichia coli strain Ec-1 was used to biosynthesize nitrated psilocybin derivative with formula (IV) from nitrated indole feedstock. The construction of Ec-1 is described in Example 3. Scaled-up culturing and material storage of engineered E. coli was conducted as described in Example 3, except that 4-nitro-5-methylindole (Combi-Blocks, www.combi-blocks.com) was used in place of 7-nitroindole. To assess product, high-resolution LC-HESI-LTQ-Orbitrap-XL MS analysis was conducted as described in Example 3. Singly protonated product with exact m/z and expected elemental formula matching the singly protonated form of N-[2-(5-methyl-4-nitro-1H-indol-3-yl)ethyl]acetamide having chemical formula (IV):

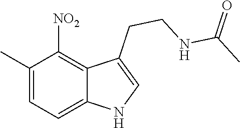 [SMILES: CC(=O)NCCc1c[nH]c2ccc(C)c([N+](=O)[O-])c12]

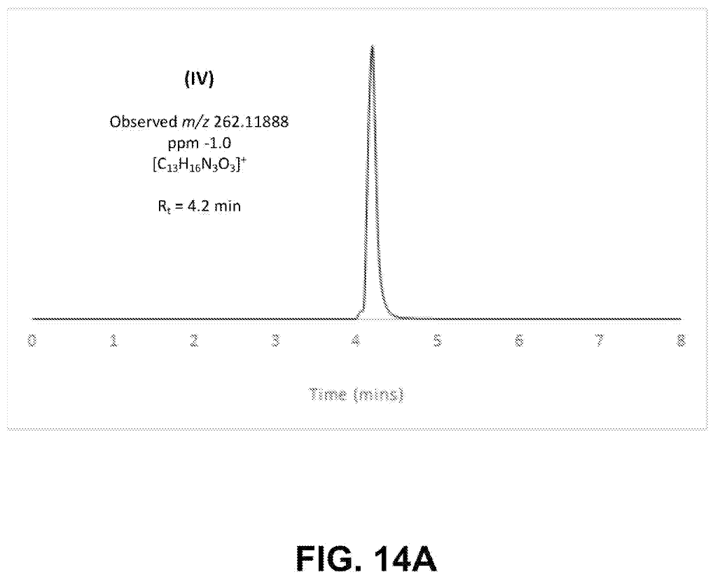

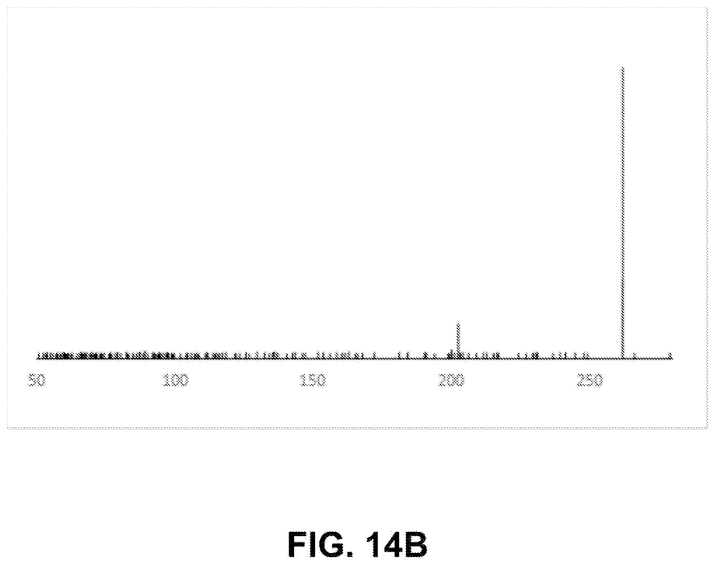

eluted at 4.2 minutes (EIC, see: FIG. 14A).

As per standard procedures (Menéndez-Perdomo et al. 2021, Mass Spectrom 56: 34683) high energy collisions (HCD) were achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of a compound of formula (IV), (FIG. 14B, Table II) (Servillo L. et al., 2013, J. Agric. Chem. 61: 5156-5162).

TABLE II
relative abundance of molecular species in a
sample containing compound (IV)

| m/z | % Relative abundance |
|---|---|
| 262.0801 | 100 |
| 202.4701 | 12 |
| 200.0832 | 3.2 |
| 88.9261 | 2.8 |
| 162.8028 | 2.5 |
| 151.7000 | 2.5 |
| 135.7864 | 2.4 |
| 129.5503 | 2.4 |
| 87.1463 | 2.4 |
| 97.1381 | 2.3 |

### Example 5—Process for Biosynthetically Making a Third Nitrated Psilocybin Derivative from Nitrated Indole Feedstock

E. coli strain Ec-2 was constructed as follows. For plasmid cloning, Top10 or XL1-blue strains were used depending on antibiotic markers. Standard LB media was used for culturing. For gene expression and feeding experiments, the parent host strain employed was BL21 (DE3). The plasmid pETM6-H10-TmTrpB-2F3-V5-BaTDC-FLAG was created as described in Example 3. This plasmid was transformed into BL21 (DE3) cells followed by ampicillin selection. Scaled-up culturing and material storage of engineered E. coli was conducted as described in Example 3, except that 4-methyl-7-nitroindole (Combi-Blocks, www.combi-blocks.com) was used in place of 7-nitroindole, and only one antibiotic was needed for selection. To assess product, high-resolution LC-HESI-LTQ-Orbitrap-XL MS analysis was conducted as described in Example 3. Singly protonated product with exact m/z and expected elemental formula matching the singly protonated form of 2-(4-methyl-7-nitro-1H-indol-3-yl)ethylamine having chemical formula (V):

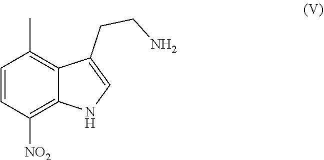 [SMILES: Cc1ccc([N+](=O)[O-])c2[nH]cc(CCN)c12]

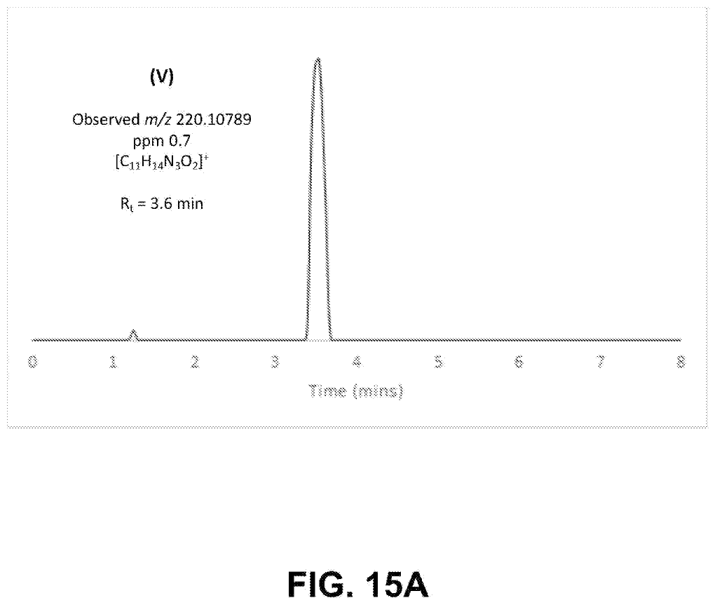

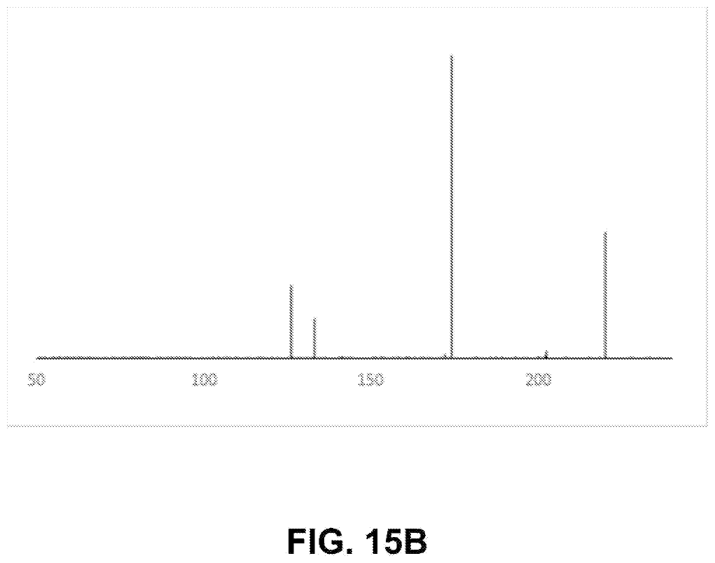

eluted at 3.6 minutes (EIC, see: FIG. 15A).

As per standard procedures (Menéndez-Perdomo et al. 2021, Mass Spectrom 56: 34683) high energy collisions (HCD) were achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of a compound of formula (V), (FIG. 15B, Table III) (Servillo L. et al., 2013, J. Agric. Chem. 61: 5156-5162).

TABLE III
relative abundance of molecular species in a
sample containing compound (IV)

| m/z | % Relative abundance |
|---|---|
| 174.0946 | 100 |
| 220.1002 | 42 |
| 126.0912 | 24 |
| 133.0316 | 12 |
| 202.4823 | 0.9 |
| 172.0967 | 1.4 |
| 200.0886 | 0.7 |
| 141.2840 | 0.6 |
| 61.0964 | 0.6 |

### Example 6—Process for Biosynthetically Making a Fourth Nitrated Psilocybin Derivative from Nitrated Indole Feedstock

Escherichia coli strain Ec-1 was used to biosynthesize nitrated psilocybin derivative with formula (VI) from nitrated indole feedstock. The construction of Ec-1 is described in Example 3. Scaled-up culturing and material storage of engineered E. coli was conducted as described in Example 3, except that 4-methyl-7-nitroindole (Combi-Blocks, www.combi-blocks.com) was used in place of 7-nitroindole. To assess product, high-resolution LC-HESI-LTQ-Orbitrap-XL MS analysis was conducted as described in Example 3. Singly protonated product with exact m/z and expected elemental formula matching the singly protonated form of N-[2-(4-methyl-7-nitro-1H-indol-3-yl)ethyl]acetamide having chemical formula (VI):

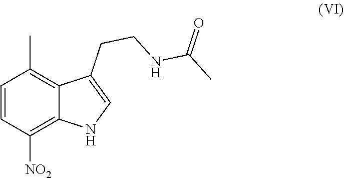 [SMILES: CC(=O)NCCc1c[nH]c2c([N+](=O)[O-])ccc(C)c12]

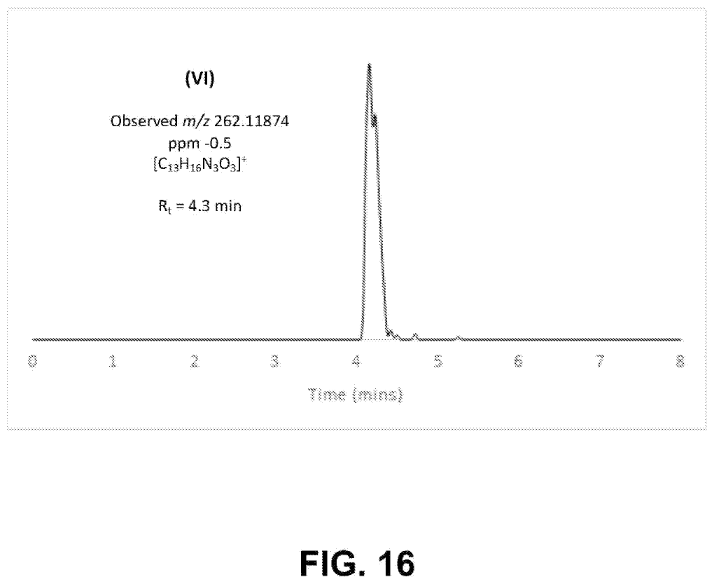

eluted at 4.3 minutes (EIC, see: FIG. 16).
As per standard procedures (Menéndez-Perdomo et al. 2021, Mass Spectrom 56: 34683) high energy collisions (HCD) were achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of a compound of formula (VI) (Servillo L. et al., 2013, J. Agric. Chem. 61: 5156-5162). Example 7—Process for biosynthetically making a fifth nitrated psilocybin derivative from nitrated Indole feedstock.

Escherichia coli strain Ec-1 was used to biosynthesize a nitrated psilocybin derivative with formula (XXVIII) from nitrated indole feedstock. The construction of Ec-1 is described in Example 3. Scaled-up culturing and material storage of engineered E. coli was conducted as described in Example 3, except that 2-methyl-6-nitroindole (Combi-Blocks, www.combi-blocks.com) was used in place of 7-nitroindole. To assess product, high-resolution LC-HESI-LTQ-Orbitrap-XL MS analysis was conducted as described in Example 3. Singly protonated product with exact m/z and expected elemental formula matching the singly protonated form of N-[2-(2-methyl-6-nitro-1H-indol-3-yl)ethyl]acetamide having chemical formula (XXVIII):

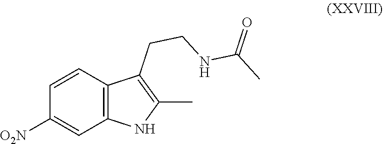 [SMILES: CC(=O)NCCc1c(C)[nH]c2cc([N+](=O)[O-])ccc12]

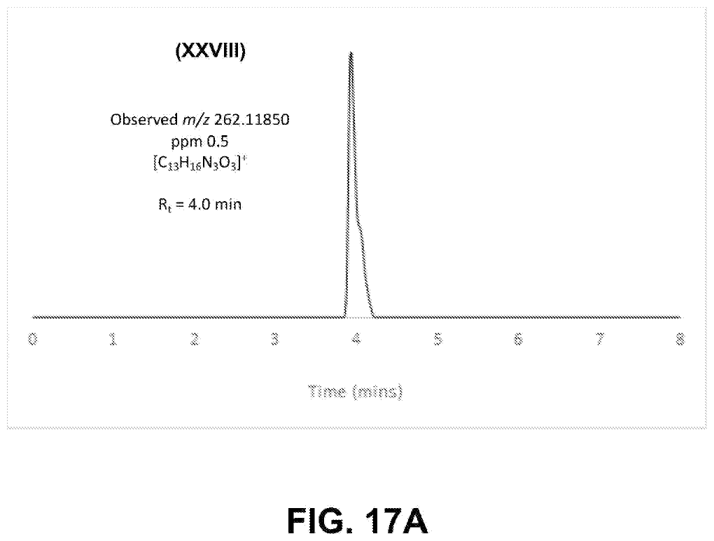

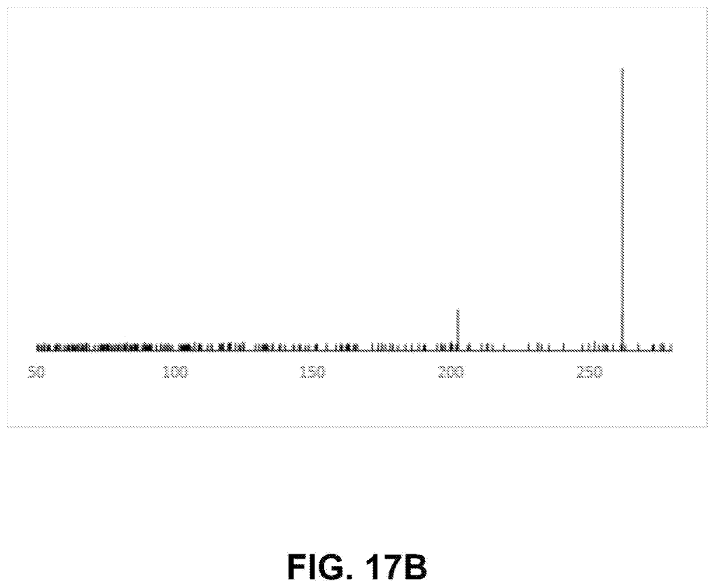

eluted at 4.0 minutes (EIC, see: FIG. 17A).

As per standard procedures (Menéndez-Perdomo et al. 2021, Mass Spectrom 56: 34683) high energy collisions (HCD) were achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of a compound of formula (VI), as follows (FIG. 17B, Table IV) (Servillo L. et al., 2013, J. Agric. Chem. 61: 5156-5162).

TABLE IV
relative abundance of molecular species in
a sample containing compound (VI)

| m/z | % Relative abundance |
|---|---|
| 262.0801 | 100 |
| 202.4776 | 15 |
| 251.9820 | 3.3 |
| 124.7317 | 3.2 |
| 200.0839 | 3.2 |
| 107.1680 | 3.0 |
| 82.5919 | 2.9 |
| 120.1858 | 2.9 |
| 231.4697 | 2.8 |
| 199.9312 | 2.8 |

1. A chemical compound or salt thereof having formula (I): 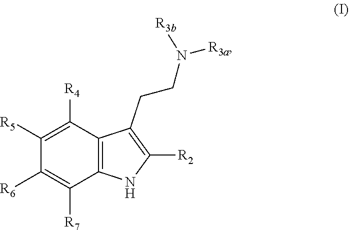
wherein at least one of R₂, R₄, R₅, R₆, or R₇ is a nitro group, and wherein (i) at least one of R₂, R₅, R₆, or R₇ is an alkyl or at least one of R₂, R₆, or R₇ is an O-alkyl, wherein R₄ when it is not nitrated is a hydrogen atom, an alkyl, O-alkyl or a hydroxy group, wherein each non-substituted R₂, R₄, R₅, R₆, or R₇ is a hydrogen atom, and wherein R₃ₐ and R_{3b} are independently a hydrogen atom, an alkyl group, an aryl group, or an acyl group; or (ii) each non-nitrated R₂, R₄, R₅, R₆, or R₇ is a hydrogen atom, a non-nitrated R₄ is a hydrogen atom or hydroxy group, and wherein R₃ₐ and R_{3b} are a hydrogen atom or an acyl group.

2. A chemical compound according to claim 1, wherein one of R₂, R₄, R₅, R₆, and R₇ is a nitro group, and one of R₂, R₄, R₆, or R₇ is an O-alkyl group.

3. A chemical compound according to claim 1, wherein one of R₂, R₄, R₅, R₆, and R₇ is a nitro group, and one of R₂, R₄, R₆, or R₇ is a (C₁-C₆)—O-alkyl group.

4. A chemical compound according to claim 1, wherein one of R₂, R₄, R₅, R₆, and R₇ is a nitro group, and one of R₂, R₄, R₆, or R₇ is a (C₁-C₆)—O-alkyl group.

5. A chemical compound according to claim 1, wherein one of R₂, R₄, R₅, R₆, and R₇ is a nitro group, and one of R₂, R₄, R₆, or R₇ is a methoxy group (—OCH₃).

6. A chemical compound according to claim 1, wherein one of R₂, R₄, R₅, R₆, and R₇ is a nitro group, and R₄, is an O-alkyl group.

7. A chemical compound according to claim 6, wherein the O-alkyl group is a (C₁-C₆)—O-alkyl group.

8. A chemical compound according to claim 6, wherein the O-alkyl group is a (C₁-C₃)—O-alkyl group.

9. A chemical compound according to claim 6, wherein the O-alkyl group is a methoxy group (—OCH₃).

10. A chemical compound according to claim 6, wherein R₇ is a nitro group.

11. A chemical compound according to claim 10, wherein the O-alkyl group is a (C₁-C₆)—O-alkyl group.

12. A chemical compound according to claim 10, wherein the O-alkyl group is a (C₁-C₃)—O-alkyl group.

13. A chemical compound according to claim 10, wherein the O-alkyl group is a methoxy group (—OCH₃).

14. A chemical compound according to claim 1, wherein one of R₂, R₄, R₅, R₆, and R₇ is a nitro group, and one of R₂, R₄, R₅, R₆, or R₇ is an alkyl group.

15. A chemical compound according to claim 1, wherein one of R₂, R₄, R₅, R₆, and R₇ is a nitro group, and one of R₂, R₄, R₅, R₆, or R₇ is a (C₁-C₆)-alkyl group.

16. A chemical compound according to claim 1, wherein one of R₂, R₄, R₅, R₆, and R₇ is a nitro group, and one of R₂, R₄, R₅, R₆, or R₇ is a (C₁-C₃) alkyl group.

17. A chemical compound according to claim 1, wherein one of R₂, R₄, R₅, R₆, and R₇ a nitro group, and one of R₂, R₄, R₅, R₆, or R₇ is a methyl group (—CH₃).

18. A chemical compound according to claim 1, wherein one of R₂, R₄, R₅, R₆, and R₇ is a nitro group, and one of R₂, R₄, R₅, R₆, or R₇ is a (C₁-C₆)-alkyl group.

19. A chemical compound according to claim 1, wherein one of R₂, R₄, R₅, R₆, and R₇ is a nitro group, and one of R₂, R₄, R₅, R₆, or R₇ is a methyl group (—CH₃).

20. A chemical compound according to claim 1, wherein in (ii) R₇ is a nitro group, and of R₂, R₄, R₅, and R₆ are a hydrogen atom.

21. A chemical compound according to claim 1, wherein two of R₂, R₄, R₅, R₆, and R₇ are a nitro group.

22. A chemical compound according to claim 1, two of R₂, R₄, R₆, and R₇ are substituents other than H, and wherein R₃ₐ and R_{3b} are each a hydrogen atom.

23. A chemical compound according to claim 1, wherein two of R₂, R₄, R₆, and R₇ are substituents other than H, one of R₃ₐ and R_{3b} is (C₁-C₆)-alkyl group and one of R₃ₐ and R_{3b} is a hydrogen atom.

24. A chemical compound according to claim 1, wherein two of R₂, R₄, R₆, and R₇ are substituents other than H, one of R₃ₐ and R_{3b} is a methyl group (—CH₃), and one of R₃ₐ and R_{3b} is a hydrogen atom.

25. A chemical compound according to claim 1, wherein two of R₂, R₄, R₆, and R₇ are substituents other than H, one of R₃ₐ and R_{3b} is a (C₁-C₆) acyl group and one of R₃ₐ and R_{3b} is a hydrogen atom.

26. A chemical compound according to claim 1, wherein two of R₂, R₄, R₆, and R₇ are substituents other than H, one of R₃ₐ and R_{3b} is a —C(═O)—CH₃ group and one of R₃ₐ and R_{3b} is a hydrogen atom.

27. A chemical compound according to claim 1, wherein the chemical compound is selected from the group consisting of compounds having formulas (III); (IV); (V); (VI); (VII); (VIII); (IX); (X); (XXVIII); and (XXIX):

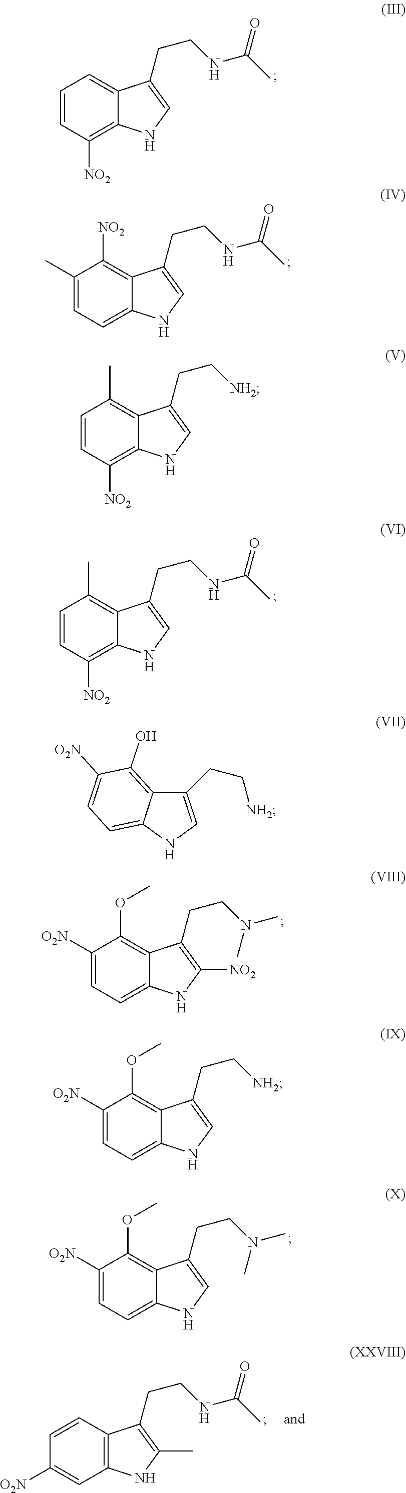

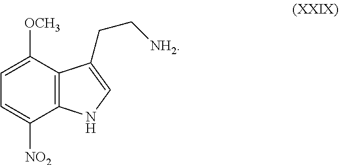 [SMILES: COc1ccc([N+](=O)[O-])c2[nH]cc(CCN)c12]

28. A chemical compound according to claim 1, wherein the compound is at least about 95% (w/w) pure.

29. A pharmaceutical drug formulation comprising an effective amount of the chemical compound of claim 1 together with a pharmaceutically acceptable excipient, diluent, or carrier.

30. A method for treating a psychiatric disorder, the method comprising administering to a subject in need thereof a pharmaceutical formulation comprising the chemical compound of claim 1, wherein the pharmaceutical formulation is administered in an effective amount to treat the psychiatric disorder in the subject.
